(19) United States
(12) Patent Application Publication
(10) Pub. No.: US 2004/0131648 A1
(43) Pub. Date: Jul. 8, 2004

(54) Nuclear hormone receptor compounds, products and methods employing same

(75) Inventors: Mitchell Anthony deLong, West Chester, OH (US); Scott Louis Cohen, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/279,397

(22) Filed: Oct. 24, 2002

(51) Int. Cl. C07D 333/08; C07D 455/06; A61K 7/00; C07C 43/18

(52) U.S. Cl. 424/401; 546/95; 554/229; 568/665; 568/445; 549/80

(57) ABSTRACT

Novel and nonobvious compounds that function, alone or in combination, as nuclear hormone receptors for the stimulation and/or improvement of murine, mammalian skin. Specifically, beta-ionol analog and fatty acid analog compounds that are believed to function as RXR, RAR and/or PPAR receptor ligands to encourage skin differentiation and discourage excess skin proliferation. The present invention further relates to one or more products, consumer and otherwise, comprising the novel, nuclear hormone receptor ligands disclosed herein. The present invention additionally seeks to encompass methods of employing both the compounds of the present invention and the products incorporating the present compounds

### FIELD OF THE INVENTION

The present invention relates to novel and nonobvious compounds that function, alone and/or in combination, as nuclear hormone receptor ligands for the stimulation and/or improvement of mammalian, and particularly human, skin. The present invention further relates to one or more products, consumer and otherwise, comprising the novel, nuclear hormone receptor ligands disclosed herein. The present invention additionally encompasses methods of employing both the compounds of the present invention and the products incorporating the present compounds.

### BACKGROUND OF THE INVENTION

Humans continue to demonstrate an obsession with appearance, particularly of the face, but increasingly of the skin and body, generally. Indeed, many believe that appearance is intrinsically linked to self-esteem, the selection of a significant other, professional advancement and overall societal acceptance. Consequently, the demand for appearance-enhancing alternatives continues to increase, as evidenced by the advent of many new products and services, each of which purports to achieve a desired appearance-enhancing result. Nevertheless, the majority of products and services that have been developed to address this growing need are designed to conceal, rather than improve, the appearance of skin. Namely, conventional solutions to this dilemma have generally sought to disguise mammalian skin imperfections with, for example, opaque chemicals that enhance only the visual appearance of skin.

Despite providing a quasi solution to the dilemma of appearance, conventional skin enhancing products have yet to address the escalation of physical ailments associated with a given skin condition. Indeed, as skin imperfections become more prevalent in humans, particularly those experiencing advanced aging, so too does the onset of physical ailments and disease. Thus, those skilled in the art have increasingly engaged in more sophisticated attempts to develop compositions that actually improve the appearance of skin, rather than simply conceal skin imperfections i.e., esse, quam videsse. Such compositions are intended to enhance the visual appearance of skin and/or address the incidence of true skin disorders, such as skin atrophy (stemming from corticosteroid administration) and post-menopausal thinning of the skin.

Notwithstanding the immense efforts exerted by those skilled in the art, little progress has been made in this realm of skin care. This limited advancement is primarily due to a lack of understanding of the processes that influence the appearance and condition of human skin. Indeed, numerous approaches in the art have generally relied upon the haphazard discovery of purported skin-enhancing alternatives, rather than the manipulation of underlying theories and synergies, to thwart the deterioration of mammalian skin. Attempts to actually improve the condition of mammalian skin have failed to address the specific and diversified needs of consumers. Consequently, consumers continue to rely upon the use and development of appearance-concealing alternatives, such as color cosmetics.

Yet a thorough understanding of the theories underlying the preservation of mammalian, and particularly human, skin has led to the surprising identification of compounds that actually have the effect of conveying true beautification and improvement benefits to mammalian skin. In particular, it has been surprisingly discovered that two particular classes of compounds are adapted to beautify and enhance the condition of mammalian skin—namely, beta-ionol analog and fatty acid analog compounds. Without wishing to be bound by theory, the compounds of the present invention are thought to function, alone and in combination, as RXR, RAR and/or PPAR nuclear hormone receptor ligands that stimulate and improve mammalian skin. The compounds of the present invention are adapted to encourage mammalian skin differentiation and discourage excess skin proliferation. Further, it has been surprisingly discovered that notable synergy is achieved via the combined administration of two or more analogs from the same or different groups of the above-described compounds.

### SUMMARY OF THE INVENTION

The present invention addresses and resolves the problems associated with the employment of conventional skin care compositions and products. To reiterate, it has been surprisingly discovered that the employment of specific, nuclear hormone receptor ligands, both individually and in combination, serves to enhance and beautify mammalian, and particularly human, skin. Indeed, the compounds of the present invention constitute an actual and viable advancement in the realm of skin care, particularly as contemporary skin care compositions have sought to simply conceal, rather than improve, the condition of mammalian skin. Specifically, it has been surprisingly discovered that the compounds of the present invention, which alter, in vitro, the partitioning of cells between a state of undifferentiated proliferation and a state of differentiation, serve to convey numerous beautification benefits to human skin, while discouraging the onset of skin disease and irritation.

Thus, in accordance with a first aspect of the present invention, novel compounds for beautifying and improving the condition of mammalian skin are disclosed. Without wishing to be bound by theory, said compounds are thought to function, alone and in combination, as nuclear hormone receptor ligands for the stimulation and/or improvement of, mammalian skin. In application, the beta-ionol analog and fatty acid analog compounds of the present invention function as ligands for RXR, RAR and/or PPAR receptors to beautify and improve the condition of mammalian skin. Specifically, the compounds disclosed herein are adapted to encourage skin differentiation and discourage excess skin proliferation upon application to mammalian skin. In another aspect of the present invention, combinations of the present compounds are employed to beautify and improve the condition of mammalian skin. Indeed, it has been surprisingly discovered that certain beta-ionol analog and fatty acid analog compounds, both of which individually demonstrate skin-enhancing activity in vitro, convey synergistic benefits upon employment in combination.

In accordance with a second aspect of the present invention, products, consumer and otherwise, incorporating the beautifying compounds of the present invention are disclosed. Such products may take an assortment of shapes and forms depending on the precise applications for which deployment of the product is desired and the needs and/or abilities of the formulator. In any instance, the products of the present invention are effective in beautifying and improving mammalian skin, by encouraging the differentiation of substrate skin and discouraging proliferation thereof. The products of the present invention, too, are adapted to convey actual skin care benefits to the substrates to which they are applied, rather than simply conceal skin imperfections like traditional skin care products.

In accordance with a third aspect of the present invention, methods of using the skin care compounds and products of the present invention are disclosed. The methods of the present invention are adapted to provide enhanced and permanent beautification benefits to mammalian, and particularly human, skin. Moreover, in another aspect of the present invention, methods of treating cancer employing the novel and nonobvious combinations of the present compounds are disclosed. As will become apparent, the compounds of the present invention, a few of which have exhibited anti-cancer activity when employed individually, provide heightened anti-cancer synergy when administered in combination. Indeed, numerous, novel synergies among traditional anti-cancer compounds (e.g., bexarotene) have been surprisingly discovered and documented via the present disclosure. The present invention further encompasses methods of treating RXR-containing mammalian tissue in need of stimulation using the compounds disclosed herein.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are, in relevant part, incorporated herein by reference. Further, while particular embodiments of the subject invention have been described, it will be apparent to those skilled in the art that various changes and modifications to the compositions disclosed herein can be made without departing from the spirit and scope of the invention.

### DETAILED DESCRIPTION OF THE INVENTION

Definitions and Usage of Terms

As used herein, “beta-ionol analog” is intended to refer to a compound that contains at least one cyclohexenyl ring, at least one gem-dimethyl group, and a side chain that contains at least one other non-ring carbon atom. Further, “beta-ionol analog” is intended to encompass compounds that contain a second or third ring, whether fused or not and whether aromatic or otherwise. Moreover, as used herein, “beta-ionol analog” is intended to encompass compounds with more than one pair of gem-dimethyl groups, such as the two pairs that characterize compounds such as bexarotene.

As used herein, “lower alkyl” is intended to refer to an acyclic chain of carbon atoms, containing from 0 to about 6 members with each member atom being optionally substituted with from 0 to about 3 substituents, each substitutent being optionally chosen from the set: hydroxy, methoxy, acetoxy, ethoxy, chloro, fluoro, bromo, thiolyl, aryl, substituted aryl, furanyl, substituted furanyl, and thiofuranyl, substituted furanyl, carboxyl, amino, a carbonyl moiety, an alkenyl moiety, an alkynyl moiety, a substituted alkenyl or alkyl moiety, with the understanding that the molecule must conform to the laws of valency for all atoms.

As used herein; “substituted” means that a member atom has one or more of its hydrogen atoms needed to ensure valency removed, and replaced by a substitutent, each substitutent optionally selected from the group consisting of: hydroxy, methoxy, acetoxy, ethoxy, chloro, fluoro, bromo, thiolyl, aryl, aryl, furanyl, furanyl, and thiofuranyl, furanyl, carboxyl, amino, a carbonyl moiety, an alkenyl moiety, an alkynyl moiety, a substituted alkenyl or alkyl moiety, with the understanding that the molecule must conform to the laws of valency for all atoms. Substituents may themselves be further substituted, so long as the total molecular weight of the molecule remains under 1000.

As used herein, “julolidine analog” is intended to refer to a compound that contains a 2,3,6,7-Tetrahydro-1H, 5H-pyrido[3,2,1-ij] quinoline moiety and containing at least one other non-ring carbon atom. Further, “julolidine analog” is intended to encompass compounds that also contain additional rings, whether merged or not and whether aromatic or otherwise.

As used herein, “fatty acid analog” is intended to encompass compounds with from about 10 to about 24 carbon atoms along a central carbon backbone. The fatty acid analogs disclosed herein comprise no more than about two functional groups, no more than about two branches or appended rings, and no more than about 25 total carbon atoms. The fatty acid analog compounds disclosed herein may be saturated or unsaturated (e.g. single or multiple unsaturations). The fatty acid analog compounds disclosed herein may be present in an oxidation state other than that of acid, such as that of an alcohol or aldehyde, or may be administered as part of an ester, amide, and/or ether.

As used herein, “beautification” is intended to encompass the reduction of fine lines, wrinkles, atrophy, texture abnormalities, hyperpigmentation and sagging to mammalian skin, as well as the overall appearance of youth and vitality.

As used herein, “cancer” is intended to encompass all diseases characterized by uncontrolled proliferation of undifferentiated cells. Specifically envisioned are cancers such as t-cell lymphoma and other leukemias, and skin cancers such as melanomas.

As used herein “skin disorders” is intended to encompass both the loss of function of the skin with age or photodamage, as well as specific conditions characterized by disturbed or dysfunctional skin. Specifically contemplated are eczematous dermatitides, allergic or contact dermatitis, phototoxic dermatitis, phytophotodematitis, radiation dermatitis, stasis dermatitis, ulcers and erosions, wounds caused by burns, cuts, trauma, bullous disorders, infection, ischemia, ichthosis, psoriasis and cutaneous atrophy, steroid induced or of unknown etiology.

As used herein, “RXR ligands” and “nuclear hormone receptor ligands” are intended to refer to compounds of the beta-ionol class, the melafleur class and the julolidine class, that inhibit, in vitro, the uncontrolled proliferation of either the HL-60 or the B16-F10 cell lines at less than or equal to 10000 micromolar, or cause, independently, or in combination with linolenic or dihomolinolenic acid, a reversal of corticosteroid-induced atrophy in the Skh-1 mouse upon topical application; or prodrugs of the same. “RXR ligands” is not intended to imply measurable binding to one of the family of nuclear hormone receptors including the RXR, RAR, PPAR, VDR, TR, ER, AR, FXR and LXR receptors of these compounds, although this is the hypothesis of the mechanism of action of these agents.

As used herein, “prodrugs” is intended to encompass all oxidation states of a ligand; e.g., an alcohol is a prodrug of a ketone or an aldehyde, as well as all commonly used biohydrolyzable groups, including but no limited to esters and amides and ketals and acetals. Prodrugs may have more than one prodrug site, and may themselves be prodrugs, as retinyl palmitate may be first cleaved to its alcohol, which is then bio-oxidized to the aldehyde and acid.

First Aspect: Compositions of Matter

In accordance with a first aspect of the present invention, novel and nonobvious compounds for permanently beautifying and improving the appearance and/or condition of mammalian skin are disclosed. Indeed, the compounds of the present invention are adapted to alter, in vitro, the partitioning of cells between a state of undifferentiated proliferation and a state of differentiation, and thus, convey numerous beautification benefits to human skin, while discouraging the onset of skin disorders. Without wishing to be bound by theory, it is believed that the ability of the present compounds to encourage skin differentiation and discourage skin proliferation serves the fundamental goal of maximizing the number of useful, productive cells, while minimizing the number of less desired, undifferentiated cells. The ability of the present compounds to maximize the number of differentiated cells, in turn, serves to increase the thickness of the mammalian skin to which they are applied. As the proportion of proliferated (i.e. undifferentiated) skin cells is minimized, the mammalian skin onto which the present compounds are applied fits more tightly around mammalian flesh and experiences reduced sagging and wrinkling as a function of time. The maximization of useful, differentiated skin cells further improves the barrier function of skin, and thus, its resistance to abrasions, cuts, sores and other physical ailments associated with poor skin conditioning. Whether employed individually or in combination, the compounds of the present invention exhibit heightened performance and synergy in the beautification and improvement of mammalian skin.

Beta-Ionol Analog Compounds

It is a fundamental goal of the present invention to identify and deploy certain beta-ionol analog compounds that are adapted to convey material beautification and improvement benefits to the mammalian skin onto which they are applied. The beta-ionol analog compounds disclosed herein constitute a particularly novel aspect of the present invention, as they are adapted to actually improve mammalian skin, rather than simply conceal skin imperfections. The present compounds, and the vast benefits achieved via their practice, further serve the fundamental goal of preventing the onset of physical ailments and irritation of the mammalian skin to which they are applied by maximizing the number of differentiated cells and improving the barrier function of skin. Thus, in accordance with a first aspect of the present invention, a beta-ionol analog compound, illustrated by the following, general structure, is disclosed:

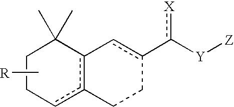

wherein “X” is a single or double bonded moiety comprising from 0 to about 12 substituted or unsubstituted carbon atoms and from 0 to about 2 heteroatoms, selected from substituted and unsubstituted, cycloalkyl or aromatic moieties of NH, S, O and combinations thereof. “Z” is a single, double, or triple bonded moiety containing from 0 to about 12 carbon atoms in a chain, optionally including a cycloalkyl or aromatic ring, both of which may be further substituted. “Y” is (CH₂)ₙ, wherein “n” is a variable having a value of from 0 to about 3. “R” is a group which may be substituted onto any ring if two or more are present and is selected from up to three independently selected substituted and unsubstituted, alkyl, cycloalkyl or aromatic moieties including CH₃, CH₂CH₃, NR₁R₂, SR, OR and combinations thereof. In another aspect of the present invention, the optical isomers, diastereomers and enantiomers of the above-depicted formula, as well as pharmaceutically acceptable salts, biohydrolyzable amides, esters, and imides thereof are encompassed as suitable skin care agents herein. Said compounds, too, exhibit enhanced beautification and improvement benefits upon application to mammalian skin, while preventing the onset of physical ailments and irritation.

In another aspect of the present invention, beta-ionol analog compounds encompassed by the above-depicted general formula and characterized by a heightened ability to inhibit the proliferation of tumor cell lines, and particularly HL-60 cells, are disclosed. Without wishing to be bound by theory, the ability of said compounds to inhibit the proliferation of tumor cell lines serves the fundamental goal of preventing the excess proliferation of undifferentiated cells. Said excess proliferation encourages the wrinkling and sagging that often typifies aging mammalian, and particularly human, skin. Further, said excess proliferation encourages the onset of cancer cell proliferation, and reduces the rate of said proliferation of established cancers.

Representative Beta-Ionol Analog Compounds

Indeed, there exists an abundance of compounds useful herein that are encompassed by the general formula set forth above in relation to the present beta-ionol analog compounds. It should be noted and underscored that the above-depicted beta-ionol analog general formula is intended to encompass obvious variations of the preferred, differentiation-inducing compounds of the present invention. The below-listed compounds are intended to serve as representative structures of the compounds that are particularly desired for use in the present invention. Other compounds that may be described by the above-listed, general formula and/or compounds that constitute obvious variations thereof are also suitable for use in the present invention.

The following non-limiting examples illustrate the compounds, compositions, and uses of the present invention. For purposes of this disclosure, the examples of suitable beta-ionol analog compounds set forth herein have been divided into the following subclasses: monocyclic core compounds, bicyclylc core compounds and tricyclic core compounds. The aforementioned sub-classes are not intended to limit the scope of the present invention. Rather, the present sub-classes have only been provided to clarify the scope of the above-depicted general structure.

TABLE I
First Sub-class of Novel, Beta-ionol analogs - Monocyclic Core Compounds

| | | |
|---|---|---|
| 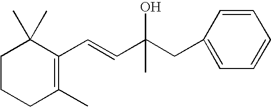 | 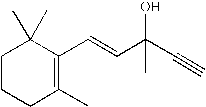 | 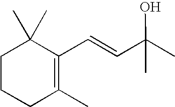 |
| 2-Methyl-1-phenyl-4- | 3-Methyl-1-(2,6,6- | 2-Methyl-4-(2,6,6- |
| (2,6,6-trimethyl- | trimethyl- | trimethyl-cyclohex- |
| cyclohex-1-enyl)-but-3- | cyclohex-1-enyl)- | 1-enyl)-but-3-en-2- |
| en-2-ol | pent-1-en-4-yn-3- | ol |
| | ol | |
| 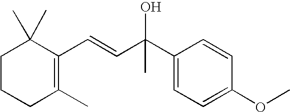 | 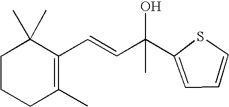 | 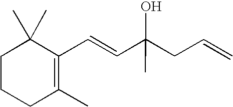 |
| 2-(4-Methoxy-phenyl)-4-(2,6,6- | 2-Thiophen-2-yl-4- | 3-Methyl-1-(2,6,6- |
| trimethyl-cyclohex-1-enyl)-but- | (2,6,6-trimethyl- | trimethyl-cyclohex- |
| 3-en-2-ol | cyclohex-1-enyl)-but- | 1-enyl)-hexa-1,5- |
| | 3-en-2-ol | dien-3-ol |
| 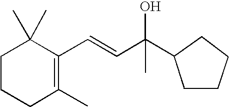 | 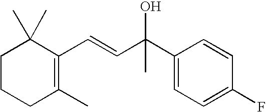 | 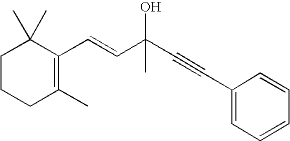 |
| 2-Cyclopentyl-4- | 2-(4-Fluoro-phenyl)-4-(2,6,6- | 3-Methyl-5-phenyl-1- |
| (2,6,6-trimethyl- | trimethyl-cyclohex-1-enyl)-but- | (2,6,6-trimethyl- |
| cyclohex-1-enyl)- | 3-en-2-ol | cyclohex-1-enyl)-pent- |
| but-3-en-2-ol | | 1-en-4-yn-3-ol |
| 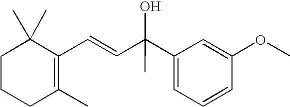 | 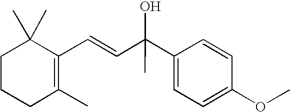 | 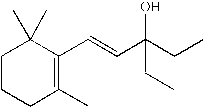 |
| 2-(3-Methoxy-phenyl)- | 2-(4-Methoxy-phenyl)- | 3-Ethyl-1-(2,6,6- |
| 4-(2,6,6-trimethyl- | 4-(2,6,6-trimethyl- | trimethyl-cyclohex-1- |
| cyclohex-1-enyl)-but- | cyclohex-1-enyl)-but-3- | enyl)-pent-1-en-3-ol |
| 3-en-2-ol | en-2-ol | |
| 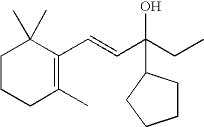 | 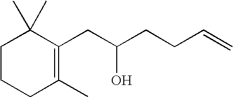 | 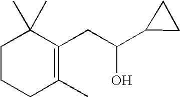 |
| 3-Cyclopentyl-1-(2,6,6- | 1-(2,6,6-Trimethyl- | 1-Cyclopropyl-2-(2,6,6- |
| trimethyl-cyclohex-1- | cyclohex-1-enyl)-hex-5- | trimethyl-cyclohex-1- |
| enyl)-pent-1-en-3-ol | en-2-ol | enyl)-ethanol |
| 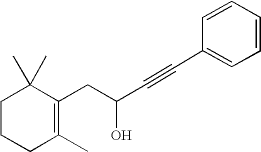 | 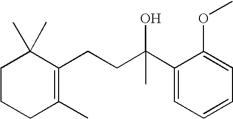 | 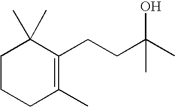 |
| 4-Phenyl-1-(2,6,6- | 2-(2-Methoxy-phenyl)- | 2-Methyl-4-(2,6,6- |
| trimethyl-cyclohex-1- | 4-(2,6,6-trimethyl- | trimethyl-cyclohex-1- |
| enyl)-but-3-yn-2-ol | cyclohex-1-enyl)- | enyl)-butan-2-ol |
| | butan-2-ol | |
| 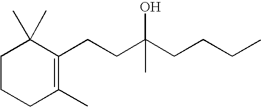 | 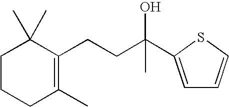 | 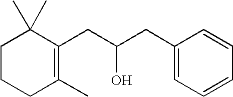 |
| 3-Methyl-1-(2,6,6- | 2-Thiophen-2-yl-4- | 1-Phenyl-3-(2,6,6- |
| trimethyl-cyclohex-1- | (2,6,6-trimethyl- | trimethyl-cyclohex-1- |
| enyl)-heptan-3-ol | cyclohex-1-enyl)- | enyl)-propan-2-ol |
| | butan-2-ol | |
| 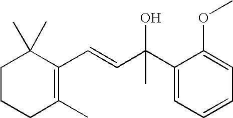 | 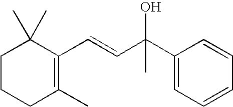 | 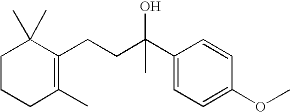 |
| 2-(2-Methoxy- | 2-Phenyl-4-(2,6,6- | 2-(4-Methoxy-phenyl)-4- |
| phenyl)-4-(2,6,6- | trimethyl-cyclohex- | (2,6,6-trimethyl-cyclohex-1- |
| trimethyl-cyclohex-1- | 1-enyl)-but-3-en-2-ol | enyl)-butan-2-ol |
| enyl)-but-3-en-2-ol | | |
| 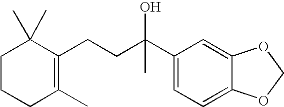 | 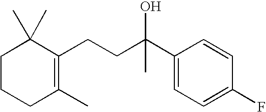 | 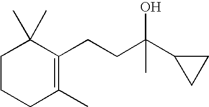 |
| 2-Benzo[1,3]dioxol-5- | 2-(4-Fluoro-phenyl)- | 2-Cyclopropyl-4- |
| yl-4-(2,6,6-trimethyl- | 4-(2,6,6-trimethyl- | (2,6,6-trimethyl- |
| cyclohex-1-enyl)-butan- | cyclohex-1-enyl)- | cyclohex-1-enyl)- |
| 2-ol | butan-2-ol | butan-2-ol |
| 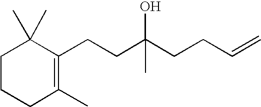 | 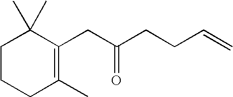 | 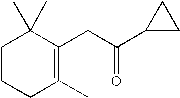 |
| 3-Methyl-1-(2,6,6-trimethyl- | 1-(2,6,6-Trimethyl- | 1-Cyclopropyl-2- |
| cyclohex-1-enyl)-hept-6- | cyclohex-1-enyl)-hex- | (2,6,6-trimethyl- |
| en-3-ol | 5-en-2-one | cyclohex-1-enyl)- |
| | | ethanone |
| 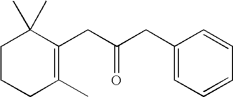 | 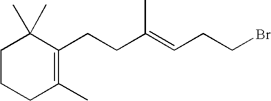 | 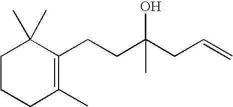 |
| 1-Phenyl-3-(2,6,6- | 2-(6-Bromo-3-methyl-hex- | 3-Methyl-1-(2,6,6- |
| trimethyl- | 3-enyl)-1,3,3-trimethyl- | trimethyl-cyclohex-1- |
| cyclohex-1-enyl)- | cyclohexane | enyl)-hex-5-en-3-ol |
| propan-2-one | | |
| 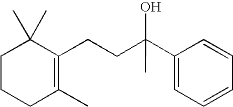 | 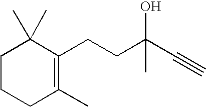 | 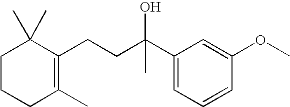 |
| 2-Phenyl-4-(2,6,6- | 3-Methyl-5-(2,6,6- | 2-(3-Methoxy-phenyl)-4- |
| trimethyl-cyclohex-1- | trimethyl- | (2,6,6-trimethyl-cyclohex-1- |
| enyl)-butan-2-ol | cyclohex-1-enyl)- | enyl)-butan-2-ol |
| | pent-1-yn-3-ol | |
| 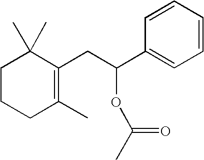 | 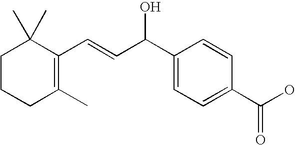 | 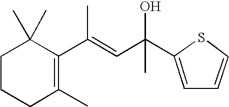 |
| Acetic acid 1-phenyl-2- | 4-[1-Hydroxy-3- | 2-Thiophen-2-yl-4- |
| (2,6,6-trimethyl- | (2,6,6-trimethyl- | (2,6,6-trimethyl- |
| cyclohex-1-enyl)-ethyl | cyclohex-1-enyl)- | cyclohex-1-enyl)- |
| ester | allyl]-benzoic acid | pent-3-en-2-ol |
| | methyl ester | |
| 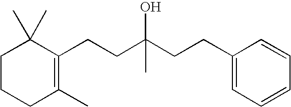 | 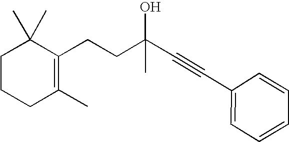 | 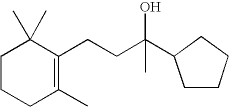 |
| 3-Methyl-1-phenyl-5-(2,6,6- | 3-Methyl-1-phenyl-5- | 2-Cyclopentyl-4- |
| trimethyl-cyclohex-1-enyl)- | (2,6,6-trimethyl- | (2,6,6-trimethyl- |
| pentan-3-ol | cyclohex-1-enyl)-pent- | cyclohex-1-enyl)- |
| | 1-yn-3-ol | butan-2-ol |
| 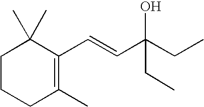 | 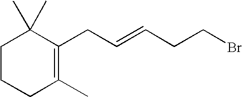 | 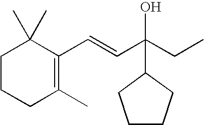 |
| 3-Ethyl-1-(2,6,6- | 2-(5-Bromo-pent-2-enyl)- | 3-Cyclopentyl-1-(2,6,6- |
| trimethyl-cyclohex-1- | 1,3,3-trimethyl- | trimethyl-cyclohex-1- |
| enyl)-pent-1-en-3-ol | cyclohexane | enyl)-pent-1-en-3-ol |
| 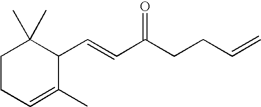 | 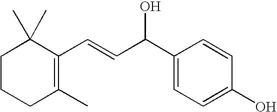 | 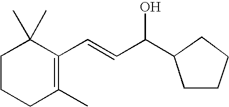 |
| 1-(2,6,6-Trimethyl- | 4-[1-Hydroxy-3-(2,6,6- | 1-Cyclopentyl-3-(2,6,6- |
| cyclohex-2-enyl)- | trimethyl-cyclohex-1- | trimethyl-cyclohex-1-enyl)- |
| hepta-1,6-dien-3-one | enyl)-allyl]-phenol | prop-2-en-1-ol |
| 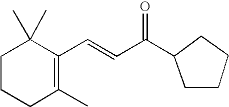 | 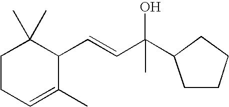 | 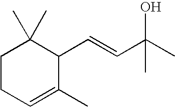 |
| 1-Cyclopentyl-3-(2,6,6- | 2-Cyclopentyl-4- | 2-Methyl-4-(2,6,6- |
| trimethyl-cyclohex-1- | (2,6,6-trimethyl- | trimethyl-cyclohex-2- |
| enyl)-propenone | cyclohex-2-enyl)-but- | enyl)-but-3-en-2-ol |
| | 3-en-2-ol | |
| 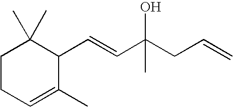 | 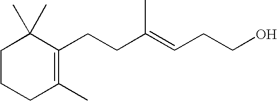 | 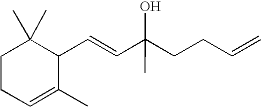 |
| 3-Methyl-1-(2,6,6- | 4-Methyl-6-(2,6,6- | 3-Methyl-1-(2,6,6- |
| trimethyl-cyclohex-2-enyl)- | trimethyl-cyclohex-1- | trimethyl-cyclohex-2- |
| hexa-1,5-dien-3-ol | enyl)-hex-3-en-1-ol | enyl)-hepta-1,6-dien- |
| | | 3-ol |
| 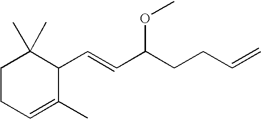 | | |
| 6-(3-Methoxy-hepta-1,6- | | |
| dienyl)-1,5,5-trimethyl- | | |
| cyclohexene | | |

TABLE II
Second Sub-class of Novel, Beta-ionol Analogs-Bicyclic Core Compounds

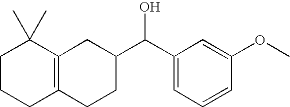

(8,8-Dimethyl- 1,2,3,4,5,6,7,8- octahydro-naphthalen- 2-yl)-(3-methoxy- phenyl)-methanol

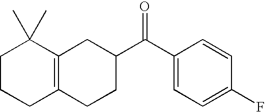

(8,8-Dimethyl- 1,2,3,4,5,6,7,8- octahydro-naphthalen- 2-yl)-(4-fluoro-phenyl)- methanone

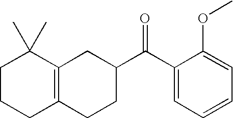

(8,8-Dimethyl-(8,8-Dimethyl- 1,2,3,4,5,6,7,8-octahydro-naphthalen-2-yl)-(2-methoxy- phenyl)-methanone

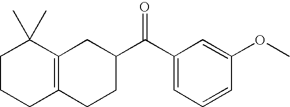

1,2,3,4,5,6,7,8- octahydro-naphthalen-2-yl)-(3- methoxy-phenyl)-methanone

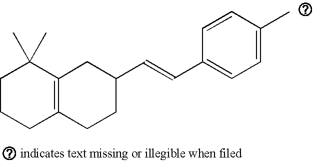

7-[3-(4-Methoxy- phenyl)-propa-1,2- dienyl]-1,1-dimethy- 1,2,3,4,5,6,7,8- octahydro-napthalene

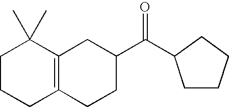

Cyclopentyl-(8,8- dimethyl- 7-[3-(4-Methoxy-phenyl)-prop-2- 1,2,3,4,5,6,7,8- octahydro-naphthalen- 2-yl)-methanone

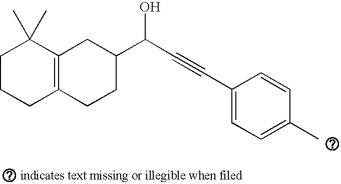

1-(8,8-Dimethyl- 1,2,3,4,5,6,7,8-octahydro-naphthalen-2-yl)-3-(4-methoxy- phenyl)-prop-2-yn-l-ol

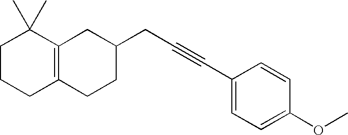

7-[3-(4-Methoxy-phenyl)-prop-2- ynyl]-1,1-dimethyl- 1,2,3,4,5,6,7,8-octahydro- naphthalene

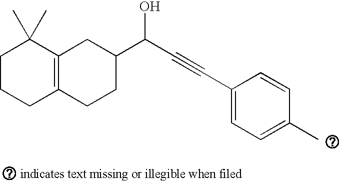

4-[3-(8,8-Dimethyl- 1,2,3,4,5,6,7,8- octahydro-naphthalen- 2-yl)-3-hydroxy-prop-1- ynyl]-phenol

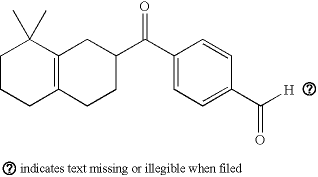

4-(8,8-Dimethyl- 1,2,3,4,5,6,7,8- octahydro- naphthalene-2- carbonyl)- benzaldehyde

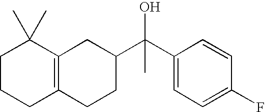

1-(8,8-Dimethyl- 1,2,3,4,5,6,7,8- octahydro-naphthalen- 2-yl)-1-(4-fluoro- phenyl)-ethanol

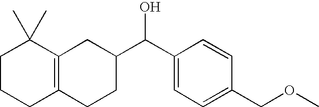

8,8-Dimethyl-1,2,3,4,5,6,7,8- octahydro-naphthalen-2-yl)-(4- methoxymethyl-phenyl)- methanol

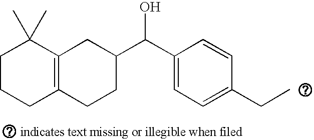

(8,8-Dimethyl- 1,2,3,4,5,6,7,8- octahydro-naphthalen- 2-yl)-(4-hydroxymethyl- phenyl)-methanol

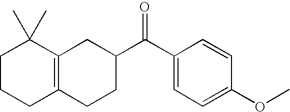

(8,8-Dimethyl- 1,2,3,4,5,6,7,8- octahydro-naphthalen- 2-yl)-(4-methoxy- phenyl)-methanone

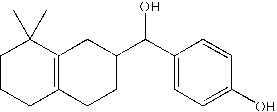

4-[(8,8-Dimethyl- 1,2,3,4,5,6,7,8- octahydro-naphthalen- 2-yl)-hydroxy-methyl]- phenol

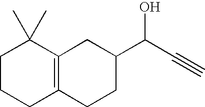

1-(8,8-Dimethyl-1 2,3,4,5,6,7,8- octahydro-naphthalen-2-yl)- prop-2-yn-1-ol

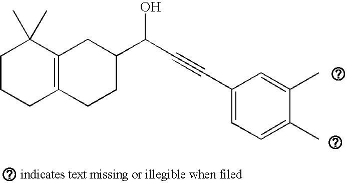

4-[3-(8,8-Dimethyl- 1,2,3,4,5,6,7,8- octahydro-naphthalen- 2-yl)-3-hydroxy-prop-1- ynyl]-fluoro benzaldehyde

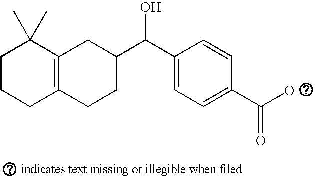

4-[(8,8-Dimethyl- 1,2,3,4,5,6,7,8- octahydro-naphthalen- 2-yl)-hydroxy-methyl]- benzoic acid isopropyl ester

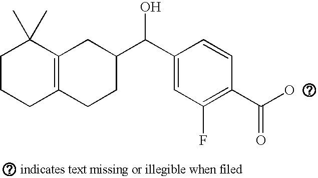

4-[(8,8-Dimethyl- 1,2,3,4,5,6,7,8- octahydro-naphthalen- 2-yl)-hydroxy-methyl]- fluoro benzoic acid

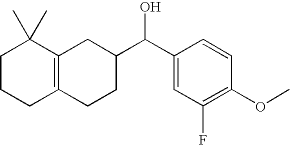

1-(8,8-Dimethyl-1,2,3,4,5,6,7,8- octahydro-naphthalen-2-yl)-1- (4-methoxy-3-fluoro-phenyl)- ethanol

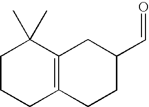

(−) Melafleur

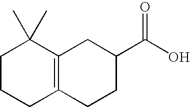

(−) Melafleur acid

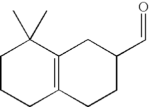

(+) Melafleur

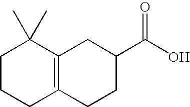

(+) Melafleur acid

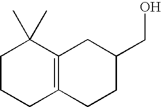

(+) Melafleur alcohol

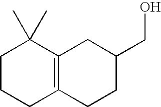

(−) Melafleur alcohol

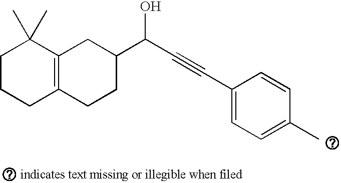

4-[3-(8,8-Dimethyl- 1,2,3,4,5,6,7,8- octahydro-naphthalen- 2-yl)-3-hydroxy-prop-1- ynyl]-benzaldehyde

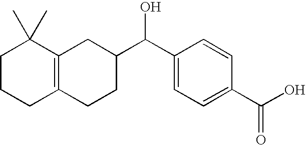

[(8,8-Dimethyl- 1,2,3,4,5,6,7,8-octahydro naphthalen-2-yl)-hydroxy- methyl]-benzoic acid

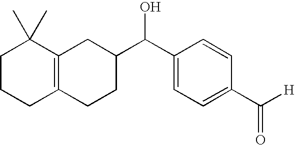

4-[(8,8-Dimethyl- 1,2,3,4,5,6,7,8- octahydro-naphthalen-2- yl)-hydroxy-methyl]- benzaldehyde

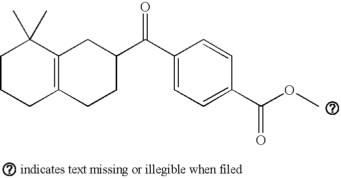

4-[(8,8-Dimethyl- 1,2,3,4,5,6,7,8- octahydro-naphthalen- 2-yl)-hydroxy-methyl]- benzoic acid methyl ester

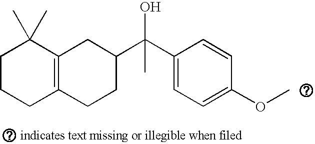

1-(8,8-Dimethyl- 1,2,3,4,5,6,7,8-octahydro-naphthalen- 2-yl)-1-(4-methoxy- phenyl)-ethanol

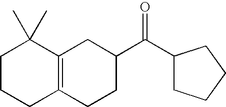

Cyclopentyl-(8,8-dimethyl- 1,2,3,4,5,6,7,8-octahydro- naphthalen-2-yl)-methanone

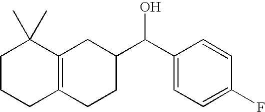

(8,8-Dimethyl- 1,2,3,4,5,6,7,8- octahydro-naphthalen-2- yl)-(4-fluoro-phenyl)- methanol

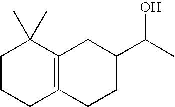

1-(8,8-Dimethyl- 1,2,3,4,5,6,7,8- octahydro-naphthalen- 2-yl)-ethanol

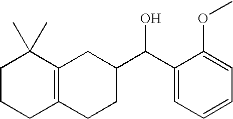

(8,8-Dimethyl- 1,2,3,4,5,6,7,8- octahydro-naphihalen- 2-yl)-(2-methoxy- phenyl)-methanol

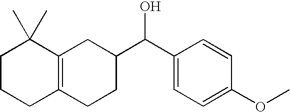

(8,8-Dimethyl- 1,2,3,4,5,6,7,8-octahydro- naphthalen-2-yl)-(4- methoxy-phenyl)-methanol

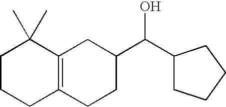

Cyclopentyl-(8,8- dimethyl- 1,2,3,4,5,6,7,8- octahydro-naphthalen- 2-yl)-methanol

TABLE III
Third Sub-class of Novel, Beta-ionol Analogs-Tricyclic Core Compounds

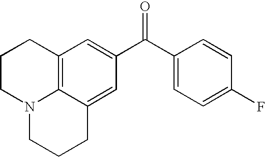

(4-Fluoro-phenyl)- (2,3,6,7-tetrahydro- 1H,5H-pyrido[3,2,1- ij]quinolin-9-yl)- methanone

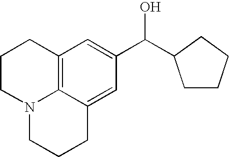

Cyclopentyl-(2,3,6,7- tetrahydro-1H,5H- pyrido[3,2,1-ij]quinolin-9- yl)-methanol

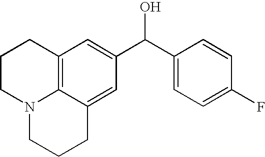

(4-Fluoro-phenyl)- (2,3,6,7-tetrahydro- 1H,5H-pyrido[3,2,1- ij]quinolin-9-yl)-methanol

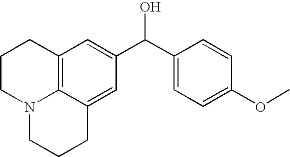

(4-Methoxy-phenyl)- (2,3,6,7-tetrahydro- 1H,5H-pyrido[3,2,1- ij]quinolin-9-yl)-methanol

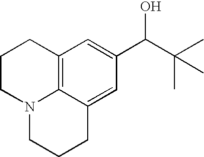

2,2-Dimethyl-1-(2,3,6,7- tetrahydro-1H,5H- pyrido[3,2,1-ij]quinolin-9- yl)-propan-1-ol

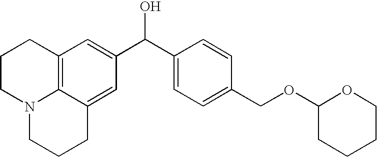

[4-(Tetrahydro-pyran-2- yloxymethyl)-phenyl]-(2,3,6,7- tetrahydro-1H,5H-pyrido[3,2,1- ij]quinolin-9-yl)-methanol

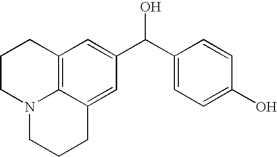

4-[Hydroxy-(2,3,6,7- tetrahydro-1H,5H- pyrido[3,2,1-ij]quinolin-9- yl)-methyl]-phenol

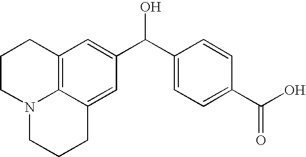

4-[Hydroxy-(2,3,6,7- tetrahydro-1H,5H- pyrido[3,2,1-ij]quinolin-9- yl)-methyl]-benzoic acid

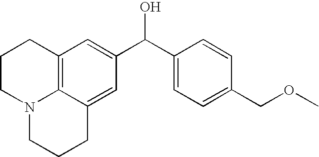

(4-Methoxymethyl- phenyl)-(2,3,6,7- tetrahydro-1H,5H- pyrido[3,2,1 -ij]quinolin-9- yl)-methanol

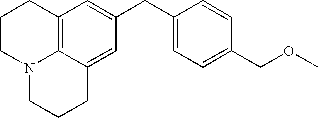

9-(4-Methoxymethyl-benzyl)- 2,3,6,7-tetrahydro-1H,5H- pyrido[3,2,1 -ij]quinoline

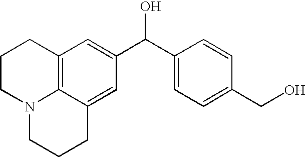

(4-Hydroxymethyl- phenyl)-(2,3,6,7- tetrahydro-1H,5H- pyrido[3,2,1 -ij]quinolin-9- yl)-methanol

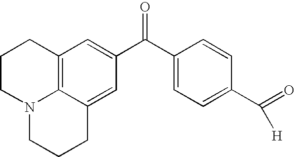

4-(2,3,6,7-Tetrahydro- 1H,5H-pyrido[3,2,1- ij]quinoline-9-carbonyl)- benzaldehyde

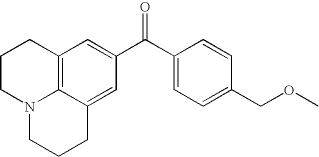

(4-Methoxymethyl- phenyl)-(2,3,6,7- tetrahydro-1H-5H- pyrido[3,2,1 -ij]quinolin- 9-yl)-methanone

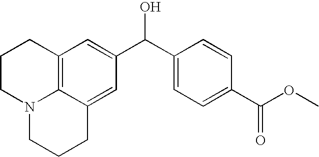

4-[Hydroxy-(2,3,6,7-tetrahydro- 1H,5H-pyrido[3,2,1-ij]quinolin- 9-yl)-methyl]-benzoic acid methyl ester

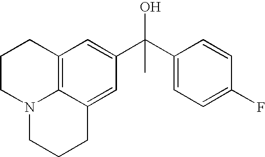

1-(4-Fluoro-phenyl)-1- (2,3,6,7-tetrahydro- 1H,5H-pyrido[3,2,1- ij]quinolin-9-yl)-ethanol

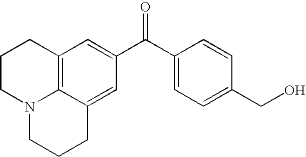

(4-Hydroxymethyl- phenyl)-(2,3,6,7- tetrahydro-1H,5H- pyrido[3,2,1-ij]quinolin-9- yl)-methanone

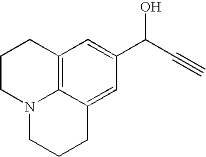

1-(2,3,6,7-Tetrahydro- 1H,5H-pyrido[3,2,1- ij]quinolin-9-yl)-prop-2-yn- 1-ol

Known Beta-ionol Compounds

There exist several other known compounds that function as beta-ionol analog compounds for use in the present invention. Said compounds may be combined with other beta-ionol analog compounds or with fatty acid analog compounds, to deliver enhanced beautification benefits to mammalian skin. Some examples of such, known beta-ionol compounds are set forth in Table IV, below:

TABLE IV
Known Beta-ionol Analogs Compounds By CAS Number

| CAS # | Name | Synonym |
|---|---|---|
| 3293-45-6 | Dihydro-□-ionol | 2-Cyclohexene-1-propanol, □,2,6,6-tetramethyl-, |
| | acetate | acetate |
| 3293-47-8 | 7,8-Dihydro-□-ionol | 1-Cyclohexene-1-propanol, □,2,6,6-tetramethyl- |
| 4361-23-3 | Tetrahydroionol | Cyclohexanepropanol, □,2,2,6- |
| | | tetramethyl- |
| 5208-93-5 | Vinyl-□-ionol | 1,4-Pentadien-3-ol, 3-methyl-1-(2,6,6- |
| | | trimethyl-1-cyclohexe n-1-yl)- |
| 6901-91-3 | Dehydro-□- | 2,4-Pentadienoic acid, 3-methyl-5-(2,6,6- |
| | ionolideneacetic acid | trimethyl-1,3-cyclohexadien-1-yl)-,(E,E)- |
| 13215-89-9 | 3,4-Dehydro-□-ionol | 3-Buten-2-ol, 4-(2,6,6-trimethyl-1,3- |
| | | cyclohexadien-1-yl)- |
| 13720-13-3 | Dihydro-□-ionol | Cyclohexanepropanol, □,2,2-trimethyl-6- |
| | | methylene- |
| 13720-37-1 | Dihydro-□-ionol | 2-Cyclohexene-1-propanol, □2,6,6- |
| | | tetramethyl- |
| 14393-44-3 | Nor-trans-□-ionol | 2-Propen-1-ol, 3-(2,6,6-trimethyl-1- |
| | | cyclohexen-1-yl)-, (E)- |
| 14398-47-1 | Ethyl dehydro-□- | 2,4-Pentadienoic acid, 3-methyl-5-(2,6,6- |
| | ionolideneacetate | trimethyl-1,3-cyclohexadien-1-yl)-, ethyl |
| | | ester, (E,E)- |
| 22030-19-9 | □-Ionol acetate | 3-Buten-2-ol, 4-(2,6,6-trimethyl-1- |
| | | cyclohexen-1-yl)-, acetate |
| 25312-34-9 | □-Ionol | 3-Buten-2-ol, 4-(2,6,6-trimethyl-2- |
| | | cyclohexen-1-yl)-, (3E)- |
| 27185-80-4 | 3-Hydroxy-□-ionol | 2-Cyclohexen-1-ol, 3-(3-hydroxy-1- |
| | | butenyl)-2,4,4-trimethyl- |
| 29790-30-5 | 3-Oxo-□-ionol | 2-Cyclohexen-1-one, 3-[(1E)-3-hydroxy- |
| | | 1-butenyl]-2,4,4-trimethyl- |
| 34318-21-3 | 3-Oxo-□-ionol | 2-Cyclohexen-1-one, 4-(3-hydroxy-1- |
| | | butenyl)-3,5,5-trimethyl- |
| 35031-11-9 | (±)-cis-□-Ionol | 3-Buten-2-ol, 4-(2,6,6-trimethyl-1- |
| | | cyclohexen-1-yl)-, (3Z)- |
| 35986-45-9 | (E)-retro-□-Ionol | 2-Butanol, 4-(2,2-dimethyl-6-methylene |
| | | cyclohexylidene)-, (E) |
| 35986-46-0 | (Z)-retro-□-Ionol | 2-Butanol, 4-(2,2-dimethyl-6-methylene |
| | | cyclohexylidene)-, (Z)- |
| 51468-87-2 | trans-□-Methylionol | 3-Buten-2-ol, 2-methyl-4-(2,6,6-trimethyl- |
| | | 1-cyclohexen-1-yl)-, (E)- |
| 54732-74-0 | Dihydro-□-ionol | 7-Oxabicyclo[4.1.0]heptane-2-propanol, |
| | epoxide | □,1,3,3-tetramethyl- |
| 58023-72-6 | 3-Hydroxy-7,8- | 3-Cyclohexen-1-ol, 4-(3-hydroxy-1- |
| | dehydro-□-ionol | butynyl)-3,5,5-trimethyl- |
| 66890-48-0 | 6,7-Dehydro-□-ionol | 3-Buten-2-ol, 4-(2,6,6-trimethyl-2- |
| | | cyclohexen-1-ylidene) |
| 70172-00-8 | Isomethyl-□-ionol | 3-Buten-2-ol, 3-methyl-4-(2,6,6-trimethyl- |
| | | 2-cyclohexen-1-yl) |
| 74352-11-7 | 9-(2-Propynyl)-□- | (E)-1-Hexen-5-yn-3-ol, 3-methyl-1-(2,6,6- |
| | ionol | trimethyl-1-cyclohexen-1-yl)- |
| 80945-23-9 | 4-Oxo-□-ionol | 3-Cyclohexen-1-one, 4-(3-hydroxy-1- |
| | | butenyl)-3,5,5-trimethyl- |
| 113110-02-4 | 3-Hydroxy-7,8- | 1-Cyclohexene-1-propanol, 3-hydroxy- |
| | dihydro-□-ionol | □,2,6,6-tetramethyl |
| 165251-48-9 | Ethynyl-retro-□-ionol | 1-Pentyn-3-ol, 3-methyl-5-(2,6,6- |
| | | trimethyl-2-cyclohexen-1-ylidene) |
| 172705-14-5 | 3-Hydroxy-5,6- | 2-Buten-1-one, 1-(4-hydroxy-2,2,6- |
| | epoxy-□-ionol | trimethyl-7-oxabicyclo[4.1.0]hept-1-yl)- |
| 256230-39-4 | □-Ionol-cade oil mixt. | 3-Buten-2-ol, 4-(2,6,6-trimethyl-2- |
| | | cyclohexen-1-yl)-, (3E)-, mixt. with |
| | | cade essential oils |
| 370591-30-3 | 3(R)-Hydroxy-5,6- | 7-Oxabicyclo[4.1.0]heptan-3-ol, 6- |
| | epoxy-□-ionol | [(1E,3R)-3-hydroxy-1-butenyl]-1,5,5- |
| | | trimethyl-, (1R,3R,6S)- |
| 79-68-5 | □-Irone | 3-Buten-2-one, 4-(2,2,3-trimethyl-6- |
| | | methylenecyclohexyl)- |
| 254899-91-7 | Ethynyl-retro-□-ionol | 1-Pentyn-3-ol, 3-methyl-5-(2,6,6- |
| | acetate | trimethyl-2-cyclohexen-1-ylidene)-, |
| | | acetate |
| 17974-59-3 | 9-Ethynyl-□-ionol | 1-Penten-4-yn-3-ol, 3-methyl-1-(2,6,6- |
| | | trimethyl-1-cyclohexen-1-yl)-, (1E) |
| 20704-59-0 | Ethylionol | 1-Penten-3-ol, 3-methyl-1-(2,6,6- |
| | | trimethyl-1-cyclohexen-1-yl)-(8Cl) |
| 79-70-9 | □-Ionone-6-methyl- | 3-Buten-2-one, 4-(2,5,6,6-tetramethyl-1- |
| | | cyclohexen-1-yl)- |
| 79-76-5 | □-Ionone | 3-Buten-2-one, 4-(2,2-dimethyl-6- |
| | | methylenecyclohexyl)- |
| 79-77-6 | (E)-□-Ionone | 3-Buten-2-one, 4-(2,6,6-trimethyl-1- |
| | | cyclohexen-1-yl)-, (3E)- |
| 79-78-7 | Allyl-□-ionone | 1,6-Heptadien-3-one, 1-(2,6,6-trimethyl-2- |
| | | cyclohexen-1-yl)- |
| 79-89-0 | □-Iraldeine | 3-Buten-2-one, 3-methyl-4-(2,6,6- |
| | | trimethyl-1-cyclohexen-1-yl)- |
| 127-41-3 | (±)-□-Ionone | 3-Buten-2-one, 4-(2,6,6-trimethyl-2- |
| | | cyclohexen-1-yl)-, (3E)- |
| 127-51-5 | □-Cetone | 3-Buten-2-one, 3-methyl-4-(2,6,6- |
| | | trimethyl-2-cyclohexen-1-yl)- |
| 1203-08-3 | 3,4-Dehydro-□- | □-Ionone, dehydro- |
| | ionone | |
| 1335-94-0 | Methylionone | Irone |
| 1337-84-4 | Delta methyl ionone | □-Ionone, methyl- |
| 4359-32-4 | □-Ionone, cyclic | 1,3-Dioxolane, 2,4-dimethyl-2-[2-(2,6,6- |
| | propylene acetal | trimethyl-2-cyclohexen-1-yl)vinyl]- |
| 5046-92-4 | Photo-□-ionone | 5H-1-Benzopyran, 6,7,8,8a-tetrahydro- |
| | | 2,5,5,8a-tetramethyl-, stereoisomer |
| 5552-30-7 | Cycloionone | 5H-1-Benzopyran, 6,7,8,8a-tetrahydro- |
| | | 2,5,5,8a-tetramethyl- |
| 6138-85-8 | Tetrahydro-□-ionone | 2-Butanone, 4-(2,2,6- |
| | | trimethylcyclohexyl)- |
| 7388-22-9 | Methyl-□-ionone | 3-Buten-2-one, 4-(2,2-dimethyl-6- |
| | | methylenecyclohexyl)-3-methyl- |
| 13720-12-2 | Dihydro-□-ionone | 2-Butanone, 4-(2,2-dimethyl-6- |
| | | methylenecyclohexyl)- |
| 13743-21-0 | Isomethyl-□-(Z)- | 3-Buten-2-one, 3-methyl-4-(2,6,6- |
| | ionone | trimethyl-2-cyclohexen-1-yl)-, (Z)- |
| 13743-48-1 | □-Ionone diethyl | Cyclohexene, 6-(3,3-diethoxy-1-butenyl)- |
| | ketal | 1,5,5-trimethyl-, (E)- |
| 14398-32-4 | □-Ionone ethylene | 1,3-Dioxolane, 2-methyl-2-[(1E)-2-(2,6,6- |
| | ketal | trimethyl-1-cyclohexen-1-yl)ethenyl]- |
| 14398-34-6 | (±)-3-Hydroxy-□- | 3-Buten-2-one, 4-(3-hydroxy-2,6,6- |
| | ionone | trimethyl-1-cyclohexen-1-yl)-, (3E)- |
| 14398-35-7 | 3,4-Didehydro-□- | 3-Buten-2-one, 4-(2,6,6-trimethyl-1,3- |
| | ionone | cyclohexadien-1-yl)-, (E)- |
| 14398-36-8 | (−)-□-Ionone | 3-Buten-2-one, 4-[(1S)-2,6,6-trimethyl-2- |
| | | cyclohexen-1-yl]-, (3E)- |
| 15764-81-5 | 1-Hydroxy-4-keto- | 2-Cyclohexen-1-one, 4-hydroxy-3,5,5- |
| | .alpha.-ionone | trimethyl-4-[(1E)-3-oxo-1-butenyl]- |
| 15789-90-9 | Isomethyl-□-(E)- | 3-Buten-2-one, 3-methyl-4-(2,6,6- |
| | ionone | trimethyl-2-cyclohexen-1-yl)- |
| 17283-81-7 | □,□-Dihydro-□- | 2-Butanone, 4-(2,6,6-trimethyl-1- |
| | ionone | cyclohexen-1-yl)- |
| 20194-68-7 | 5-keto-□-Ionone | 2-Cyclohexen-1-one, 3,5,5-trimethyl-4-(3- |
| | | oxo-1-butenyl)- |
| 20483-36-7 | Dihydrodehydro-□- | 2-Butanone, 4-(2,6,6-trimethyl-1,3- |
| | ionone | cyclohexadien-1-yl)- |
| 23069-12-7 | 3-Ethoxy-3,4- | -Buten-2-one, 4-(4-ethoxy-2,6,6-trimethyl- |
| | dehydro-□-ionone | 1,3-cyclohexadien-1-yl)- |
| 23267-57-4 | □-Ionone 5,6- | 3-Buten-2-one, 4-(2,2,6-trimethyl-7- |
| | epoxide | oxabicyclo[4.1.0]hept-1-yl)- |
| 24190-29-2 | (+)-(6R)-□-Ionone | 3-Buten-2-one, 4-[(1R)-2,6,6-trimethyl-2- |
| | | cyclohexen-1-yl]-, (3E)- |
| 24190-32-7 | (−)-□-Ionone | (3E) 3-Buten-2-one, 4-[(1R)-2,2-dimethyl- |
| | | 6-methylenecyclohexyl]- |
| 24190-33-8 | Dihydro-□-ionone | CN 2-Butanone, 4-[(1S)-2,2-dimethyl-6- |
| | | methylenecyclohexyl]- |
| 27185-77-9 | 3-Keto-□-ionone | 2-Cyclohexen-1-one, 2,4,4-trimethyl-3-(3- |
| | | oxo-1-butenyl)- |
| 27417-37-4 | □-Ionone, methyl- | 3-Buten-2-one, 4-(2,2-dimethyl-6- |
| | | methylenecyclohexyl)-, monomethyl |
| | | deriv. |
| 28418-08-8 | 1-Hydroxy-4-oxo-□- | 2-Cyclohexen-1-one, 4-hydroxy-3,5,5- |
| | ionone | trimethyl-4-(3-oxo-1-butenyl)- |
| 28494-34-0 | trans-5,6-Dihydro- | 3-Buten-2-one, 4-(1,2-dihydroxy-2,6,6- |
| | 5,6-dihydroxy-□- | trimethylcyclohexyl)- |
| | ionone | |
| 29790-29-2 | (E)-3-Oxo-□-ionone | 2-Cyclohexen-1-one, 2,4,4-trimethyl-3- |
| | | [(1E)-3-oxo-1-butenyl]- |
| 31499-72-6 | 3,4-Dihydro-□- | 2-Butanone, 4-(2,6,6-trimethyl-2- |
| | ionone | cyclohexen-1-yl)- |
| 31798-12-6 | I-□-Ionone | 3-Buten-2-one, 4-(2,6,6-trimethyl-2- |
| | | cyclohexen-1-yl)-, (−)- |
| 32210-22-3 | Cyclic □-ionone | Benzopyran-(□-ionone) cyclic ether |
| 35031-06-2 | (Z)-□-Ionone | 3-Buten-2-one, 4-(2,6,6-trimethyl-1- |
| | | cyclohexen-1-yl)-, (3Z)- |
| 35896-32-3 | threo-Epoxy-□- | Ethanone, 1-[3-(2,2-dimethyl-6- |
| | ionone | methylenecyclohexyl)oxiranyl]- |
| 35986-43-7 | (E)-retro-□-Ionone | 2-Butanone, 4-(2,2-dimethyl-6- |
| | | methylenecyclohexylidene)- |
| 35986-44-8 | cis-retro-□-Ionone | 2-Butanone, 4-(2,2-dimethyl-6- |
| | | methylenecyclohexylidene)-, (4Z)- |
| 36340-49-5 | (E)-□-Ionone | 3-Buten-2-one, 4-(2,2,6-trimethyl-7- |
| | epoxide | oxabicyclo[4.1.0]hept-1-yl)-, (3E)- |
| 37079-64-4 | □,□-Epoxy-□-ionone | Ethanone, 1-[3-(2,6,6-trimethyl-2- |
| | | cyclohexen-1-yl)oxiranyl]- |
| 37665-95-5 | (±)-cis-3-Methoxy-□- | 3-Buten-2-one, 4-(4-methoxy-2,6,6- |
| | ionone | trimethyl-2-cyclohexen-1-yl)-, |
| | | [1□(E),4□]- |
| 37665-96-6 | (±)-trans-3-Methoxy- | 3-Buten-2-one, 4-(4-methoxy-2,6,6- |
| | □-ionone | trimethyl-2-cyclohexen-1-yl)-, |
| | | [1□(E),4□]- |
| 37677-81-9 | 3,4-Epoxy-□-ionone | 3-Buten-2-one, 4-(1,3,3-trimethyl-7- |
| | | oxabicyclo[4.1.0]hept-2-yl)- |
| 37677-82-0 | cis-4-keto-□-Ionone | 2-Cyclohexen-1-one, 2,4,4-trimethyl-3-(3- |
| | | oxo-1-butenyl)-, (Z)- |
| 38274-01-0 | 3-Hydroxy-5,6- | Buten-2-one, 4-(4-hydroxy-2,2,6-tri |
| | epoxy-□-ionone | methyl-7-oxabicyclo[4.1.0]hept-1-yl)- |
| 38274-02-1 | 3-Hydroxy-5,6- | 3-Buten-2-one, 4-[4-(acetyloxy)-2,2,6- |
| | epoxy-□-ionone | trimethyl-7-oxabicyclo[4.1.0]hept-1-yl] |
| | acetate | |
| 38963-23-4 | 4′-Methoxyepoxy-□- | 3-Buten-2-one, 4-(4-methoxy-2,2,6- |
| | ionone | trimethyl-7-oxabicyclo [4.1.0]hept-1-yl)- |
| 38963-37-0 | 4′-Hydroxy-1′,2′- | 3-Buten-2-one, 4-(4-hydroxy-2,2,6- |
| | dihydro-□-ionone | trimethylcyclohexyl)- |
| 39190-05-1 | □-Ionone oxime | 3-Buten-2-one, 4-(2,6,6-trimethyl-1- |
| | | cyclohexen-1-yl)-, oxime |
| 39190-15-3 | □-Isomethylionone | 3-Buten-2-one, 3-methyl-4-(2,6,6- |
| | oxime | trimethyl-2-cyclohexen-1-yl)-, oxime |
| 39721-65-8 | (+)-Dihydro-□-ionone | 2-Butanone, 4-(2,6,6-trimethyl-2- |
| | | cyclohexen-1-yl)-, (R)- |
| 39900-23-7 | 7,8-Epoxydihydro-□- | Ethanone, 1-[3-(2,6,6-trimethyl-2- |
| | ionone | cyclohexen-1-yl)oxiranyl]-, |
| | | [2□,3□(R*)]- |
| 39900-24-8 | threo-7,8- | Ethanone, 1-[3-(2,2-dimethyl-6- |
| | Epoxydihydro-□- | methylenecyclohexyl)oxiranyl]-, |
| | ionone | [2□,3□(R*)]- |
| 39900-25-9 | erythro-7,8- | Ethanone, 1-[3-(2,2-dimethyl-6- |
| | Epoxydihydro-□- | methylenecyclohexyl)oxiranyl]-, |
| | ionone | [2□,3□(S*)]- |
| 49816-69-5 | (±)-□-Ionone | 3-Buten-2-one, 4-(2,2-dimethyl-6- |
| | | methylenecyclohexyl)-, (3E)- |
| 49816-95-7 | (+)-3-Oxo-□-ionone | [R-(E)]-2-Cyclohexen-1-one, 3,5,5- |
| | | trimethyl-4-(3-oxo-1-butenyl)-, |
| 50281-38-4 | (3R)-3-Hydroxy-□- | 3-Buten-2-one, 4-[(4R)-4-hydroxy-2,6,6- |
| | ionone | trimethyl-1-cyclohexen-1-yl]-, |
| | | (3E) |
| 51595-85-8 | trans-5,6-Epoxy-8- | 3-Buten-2-one, 3-methyl-4-(2,2,6- |
| | methyl-□-ionone | trimethyl-7-oxabicyclo[4.1.0]hept-1-yl)-, |
| | | (E)- |
| 51703-99-2 | (E)-8-Methyl-□- | 3-Buten-2-one, 3-methyl-4-(2,6,6- |
| | ionone | trimethyl-1-cyclohexen-1-yl)-, (E)- |
| 52612-53-0 | (+)-cis- | 2-Butanone, 4-(2,2,6- |
| | Tetrahydroionone | trimethylcyclohexyl)-, (1S-trans)- |
| 53798-34-8 | (−)-3-Oxo-□-ionone | 2-Cyclohexen-1-one, 3,5,5-trimethyl-4- |
| | | (3-oxo-1-butenyl)-, [S-(E)]- |
| 54685-98-2 | (E)-Dehydro-□- | (E)-Buten-2-one 4-(2,2,6-trimethyl-7- |
| | ionone epoxide | oxabicyclo[4.1.0]hept-4-en-1-yl) |
| 55093-41-9 | E-retro-□-Ionone | 2-Butanone, 4-(2,6,6-trimethyl-2- |
| | | cyclohexen-1-ylidene)-, (4E)- |
| 56052-61-0 | retro-□-Ionone | 2-Butanone, 4-(2,6,6-trimethyl-2- |
| | | cyclohexen-1-ylidene)- |
| 56782-84-4 | trans-Dihydroionone | 3-Buten-2-one, 4-(2,2,6- |
| | | trimethylcyclohexyl)-, trans- |
| 57461-18-4 | 2-Oxo-3,4- | 2,4-Cyclohexadien-1-one, 4,6,6-trimethyl- |
| | didehydro-□-ionone | 5-(3-oxo-1-butenyl)-, (E)- |
| 57461-19-5 | 2-Oxo-□-ionone | 3-Cyclohexen-1-one, 2,2,4-trimethyl-3- |
| | | [(1E)-3-oxo-1-butenyl] |
| 63429-28-7 | □-Methylionone | 1-Penten-3-one, 1-(2,6,6-trimethyl-1- |
| | | cyclohexen-1-yl)-, (1E)-(9Cl) |
| 67504-50-1 | (S)-2-Hydroxy-□- | 3-Buten-2-one, 4-[(5S)-5-hydroxy-2,6,6- |
| | ionone | trimethyl-1-cyclohexen-1-yl]-, |
| 68480-17-1 | Dihydromethyl-□- | 3-Pentanone, 1-(2,6,6-trimethyl-2- |
| | ionone | cyclohexen-1-yl)- |
| 71629-13-5 | (−)-4-Hydroxy-□- | 3-Buten-2-one, 4-(3-hydroxy-2,6,6- |
| | ionone | trimethyl-1-cyclohexen-1-yl)-, [R-(E)]- |
| 71629-15-7 | (+)-4-Hydroxy-□- | 3-Buten-2-one, 4-(3-hydroxy-2,6,6- |
| | ionone | trimethyl-1-cyclohexen-1-yl)-, [S-(E)]- |
| 72008-46-9 | 4-Oxo-□- | 2-Cyclohexen-1-one, 2,4,4-trimethyl-3-(3- |
| | dihydroionone | oxobutyl)- |
| 72117-72-7 | Dimethylionone | 1-Penten-3-one, 2-methyl-1-(2,6,6- |
| | | trimethyl-2-cyclohexen-1-yl)- |
| 74345-31-6 | 3-Acetoxy-□-ionone | (3E)-3-Buten-2-one,4-[3-(acetyloxy)- |
| | | 2,6,6-trimethyl-1-cyclo hexen-1-yl] |
| 79734-43-3 | 3-Oxo-□-ionone | 2-Cyclohexen-1-one, 3,5,5-trimethyl-4- |
| | | [(1E)-3-oxo-1-butenyl]- |
| 88160-79-6 | Ionone, (2-propenyl) | |
| 89128-16-5 | (Z)-7-Methyl-□- | 3-Penten-2-one, 4-(2,6,6-trimethyl-1- |
| | ionone | cyclohexen-1-yl)-, (Z)- |
| 89128-17-6 | (E)-7-Methyl-□- | 3-Penten-2-one, 4-(2,6,6-trimethyl-1- |
| | ionone | cyclohexen-1-yl)-, |
| 91387-66-5 | (+)-3-Hydroxy-7,8- | (+)-3-Butyn-2-one, 4-(4-hydroxy-2,6,6- |
| | dehydro-□-ionone | trimethyl-1-cyclohexen-1-yl) |
| 92510-04-8 | cis-Sesqui-□-ionone | 3-Buten-2-one, 4-[2,6,6-trimethyl-5-(3- |
| | | methyl-2-butenyl)-2-cyclohexen-1-yl]-, |
| | | [1□(E),5□]- |
| 92620-17-2 | trans-Sesqui-□- | 3-Buten-2-one, 4-[2,6,6-trimethyl-5-(3- |
| | ionone | methyl-2-butenyl)-2-cyclohexen-1-yl]-, |
| | | [1□(E),5□]- |
| 93302-56-8 | alpha.-Methylionone | 1-Penten-3-one, 1-(2,6,6-trimethyl-2- |
| | | cyclohexen-1-yl)-, (1E)- |
| 98633-46-6 | (±)-2,3-Dihydro-□- | 3-Buten-2-one, 4-(2,2,6- |
| | ionone | trimethylcyclohexyl)-, (3E)- |
| 116296-75-4 | 4-Hydroxy-□-ionone | 3-Buten-2-one, 4-(4-hydroxy-2,6,6- |
| | | trimethyl-1-cyclohexen-1-yl)-, (3E)- |
| 117048-10-9 | 4-Oxo-□-ionone | 3-Cyclohexen-1-one, 3,5,5-trimethyl-4- |
| | | [(1E)-3-oxo-1-butenyl]- |
| 122258-61-1 | 3-Methoxy-□-ionone | 3-Buten-2-one, 4-(3-methoxy-2,6,6- |
| | | trimethyl-1-cyclohexen-1-yl)-, |
| 133692-87-2 | 3-Butoxy-□-ionone | 3-Buten-2-one, 4-(3-butoxy-2,6,6- |
| | | trimethyl-1-cyclohexen-1-yl)-, (E)- |
| 133692-88-3 | 3-(Benzyloxy)-□- | 3-Buten-2-one, 4-[2,6,6-trimethyl-3- |
| | ionone | (phenylmethoxy)-1-cyclohexen-1-yl]-, (E)- |
| 141441-04-5 | □-Ionone (9Cl) | |
| 157552-20-0 | Iso-□-ionone | 3-Buten-2-one, 4-(2,3,3-trimethyl-1- |
| | | cyclohexen-1-yl)-, (E)- |
| 79-69-6 | □-Ionone, methyl | 3-Buten-2-one, 4-(2,5,6,6-tetramethyl-2- |
| | | cyclohexen-1-yl)- |
| 22029-76-1 | □-Ionol | 3-Buten-2-ol, 4-(2,6,6-trimethyl-1- |
| | | cyclohexen-1-yl)-(8Cl, 9Cl) |
| 5208-92-4 | Vinyl-□-ionol | 1,4-Pentadien-3-ol, 3-methyl-1-(2,6,6- |
| | | trimethyl-2-cyclohexe-1-yl)- |
| 38758-05-3 | Methyl melafleur | |
| | alcohol | |
| | Melafleur | 2-Naphthalenecarboxaldehyde, |
| | | 1,2,3,4,5,6,7,8-octahydro |
| 59175-66-5 | Melafleur acid methyl | 2-Naphthalenecarboxylic acid, |
| | ester | 1,2,3,4,5,6,7,8-octahydro-8,8-dimethyl-, |
| | | methyl ester |
| 59175-65-4 | Butenyl Melafleur | 4-Penten-1-one, 1-(1,2,3,4,5,6,7,8- |
| | | octahydro-8,8-dimethyl-2-naphthalenyl)- |
| 92860-49-6 | Alpha-methyl | 2-Naphthalenecarboxylic acid, |
| | melafleuric acid | 1,2,3,4,5,6,7,8-octahydro-2,8,8-trimethyl-, |
| | methyl ester | methyl ester |
| | Melafleur | 2-Naphthalenecarboxylic acid, 3-[(2,3- |
| | indoylyamide | dihydro-1H-indol-1-yl)carbonyl]- |
| 431075-04-6 | | 2--Naphthalene carboxylic acid, |
| | | 1,2,3,4,5,6,7,8-octahydro-8,8-dimethyl- |
| | | 3-[[(3-pyridinylmethyl) amino]carbonyl]- |
| 371124-04-8 | Melafleur oxime | Ethanone, 1-(1,2,3,4,5,6,7,8-octa hydro- |
| | | 8,8-dimethyl-2-naphthalenyl)-,oxime |
| 182630-49-5 | | 2-Naphthalenecarboxylic acid, 6-[[(1,1- |
| | | dimethylethyl) dimethylsilyl]oxy]- |
| | | 1,2,3,4,5,6,7,8-octahydro-8,8-dimethyl-, |
| | | methyl ester |
| 107620-98-4 | | 1-Penten-3-one, 1-(1,2,3,4,5,6,7,8- |
| | | octahydro-8,8-dimethyl-2-naphthyl)- |
| 93804-62-7 | Melafleur alcohol | 2-Naphthalenemethanol, 1,2,3,4,5,6,7,8- |
| | | octahydro-8,8-dimethyl- |
| 412314-45-5 | 1-methyl melafleur | |
| | acid ethyl ester | |
| 101262-17-3 | | 1,3-Dioxolane, 2-(1,2,3,4,5,6,7,8- |
| | | octahydro-8,8-dimethyl-2-naphthyl)- |
| 72928-51-9 | Melafleur nitrile | 2-Naphthalenecarbonitrile, |
| | | 1,2,3,4,5,6,7,8-octahydro-8,8-dimethyl- |
| 67746-27-4 | Melafleur acetone | Ethanone, 1-(1,2,3,4,5,6,7,8-octahydro- |
| | | 8,8-dimethyl-2-naphthalenyl)- |
| 133192.50.4 | Melafleur ethyl | Propanone, 1-(1,2,3,4,5,6,7,8-octahydro- |
| | ketone | 8,8-dimethyl-2-naphthalenyl)- |
| 265103-60-4 | 7-methyl melafleur | Ethanone, 1-(1,2,3,4,5,6,7,8-octahydro- |
| 265103-59-1 | ketone | 7,8,8-trimethyl-2-naphthalenyl)- |
| 101271-26-5 | | 3-Buten-2-one, 4-(1,2,3,4,5,6,7,8- |
| | | octahydro-8,8-dimethyl-2-naphthyl)- |
| 105520-04-5 | | 2-Naphthol, 1,2,3,4,5,6,7,8-octahydro- |
| | | 8,8-dimethyl- |

TABLE IV
Known Compounds by CAS Number

| CAS Number | Synonym |
|---|---|
| 33985-71-6 | Julolidine-9-carboxaldehyde |
| | 2,3,6,7-Tetrahydro-1H,5H-pyrido[3,2,1-ij]quinoline-9-carbaldehyde |
| 101077-18-3 | Julolidine-9-methanol |
| | (2,3,6,7-Tetrahydro-1H,5H-pyrido[3,2,1-ij]quinolin-9-yl)-methanol |
| | 1H,5H-Benzo[ij]quinolizine-9-methanol, 2,3,6,7-tetrahydro-(9Cl) |
| 107070-67-7 | Benzoic acid, 2-[(2,3,6,7-tetrahydro-10-hydroxy-1H,5H-benzo[ij]quinolizin-9-yl)carbonyl]-(9Cl) |
| 109055-39-2 | 1H,5H-Benzo[ij]quinolizine, 2,3,6,7-tetrahydro-9-oxazolo[4,5-b]pyridin-2-yl-(9Cl) |
| 113139-17-6 | 1H,5H-Benzo[ij]quinolizine-9-carboxylic acid, 2,3,6,7-tetrahydro-1,7-dioxo-, ethyl ester (9Cl) |
| 113139-18-7 | 1H,5H-Benzo[ij]quinolizine-9-carboxylic acid, 2,3,6,7-tetrahydro-2,6-dimethyl-1,7-dioxo-, ethyl ester |
| | (9Cl) |
| 113139-19-8 | 1H,5H-Benzo[ij]quinolizine-9-carboxylic acid, 2,3,6,7-tetrahydro-1,7-dioxo-, methyl ester (9Cl) |
| 113139-20-1 | 1H,7H-Benzo[ij]quinolizine-1,7-dione, 9-acetyl-2,3,5,6-tetrahydro-(9Cl) |
| 115497-51-3 | Ethanone, 1-(2,3,6,7-tetrahydro-1H,5H-benzo[ij]quinolizin-9-yl)-(9Cl) |
| 115655-28-2 | 1(3H)-Isobenzofuranone, 3-[2,2-bis[4-(dimethylamino)phenyl]ethenyl]-4,5,6,7-tetrachloro-3-(2,3,6,7- |
| | tetrahydro-1H,5H-benzo[ij]quinolizin-9-yl)-(9Cl) |
| 117491-83-5 | 2,5-Pyrrolidinedione, 1-[[(2′,3′,6′,7′,12′,13′,16′,17′-octahydro-3-oxospiro[isobenzofuran-1(3H),9′- |
| | [1H,5H,9H,11H,15H]xantheno[2,3,4-ij:5,6,7-i′j′]diquinolizin]-6-yl)carbonyl]oxy]-(9Cl) |
| 117599-35-6 | 2,5-Pyrrolidinedione, 1-[[(2′,3′,6′,7′,12′,13′,16′,17′-octahydro-3-oxospiro[isobenzofuran-1(3H),9′- |
| | [1H,5H,9H,11H,15H]xantheno[2,3,4-ij:5,6,7-i′j′]diquinolizin]-7-yl)carbonyl]oxy]-(9Cl) |
| 120530-78-1 | 1H,5H-Benzo[ij]quinolizine, 9-(2-benzoxazolyl)-2,3,6,7-tetrahydro-(9Cl) |
| 120530-79-2 | 1H,5H-Benzo[ij]quinolizine, 2,3,6,7-tetrahydro-9-naphth[1,2-d]oxazol-2-yl-(9Cl) |
| 131071-63-1 | 1,3-Benzenedicarboxylic acid, 4-[(2,3,6,7-tetrahydro-8-hydroxy-1H,5H-benzo[ij]quinolizin-9- |
| | yl)carbonyl]-(9Cl) |
| 132092-34-3 | 2H,5H,7H,11H-Pyrano[3′,2′:3,4][1]benzopyrano[6,7,8-ij]quinolizine-2,5-dione, 4-chloro-8,9,12,13- |
| | tetrahydro-(9Cl) |
| 134036-21-8 | 1(3H)-lsobenzofuranone, 3-[4-[4-(dimethylamino)phenyl]-4-phenyl-1,3-butadienyl]-3-(2,3,6,7- |
| | tetrahydro-1H,5H-benzo[ij]quinolizin-9-yl)-(9Cl) |
| 134581-68-3 | 1H,5H-Benzo[ij]quinolizine-9-methanol, ?-ethyl-2,3,6,7-tetrahydro-(9Cl) |
| 134581-69-4 | 1-Propanone, 1-(2,3,6,7-tetrahydro-1H,5H-benzo[ij]quinolizin-9-yl)-(9Cl) |
| 134581-70-7 | 1-Propanone, 2-bromo-1-(2,3,6,7-tetrahydro-1H,5H-benzo[ij]quinolizin-9-yl)-(9Cl) |
| 136878-31-4 | Ethanone, 1-(2,3,6,7-tetrahydro-8-hydroxy-1H,5H-benzo[ij]quinolizin-9-yl)-(9Cl) |
| 136878-32-5 | Ethanone, 1-(2,3,6,7-tetrahydro-8-hydroxy-10-methyl-1H,5H-benzo[ij]quinolizin-9-yl)-(9Cl) |
| 136878-33-6 | Ethanone, 1-(8-hexyl-2,3,6,7-tetrahydro-10-hydroxy-1H,5H-benzo[ij]quinolizin-9-yl)-(9Cl) |
| 136878-34-7 | Ethanone, 1-(2,3,6,7-tetrahydro-8-hydroxy-10-phenyl-1H,5H-benzo[ij]quinolizin-9-yl)-(9Cl) |
| 136878-35-8 | Ethanone, 1-[2,3,6,7-tetrahydro-8-hydroxy-10-(phenylmethyl)-1H,5H-benzo[ij]quinolizin-9-yl]-(9Cl) |
| 136878-36-9 | Ethanone, 1-[2,3,6,7-tetrahydro-8-hydroxy-10-(octyloxy)-1H,5H-benzo[ij]quinolizin-9-yl]-(9Cl) |
| 136878-37-0 | Ethanone, 1-[2,3,6,7-tetrahydro-8-hydroxy-10-(trifluoromethyl)-1H,5H-benzo[ij]quinolizin-9-yl]-(9Cl) |
| 136878-38-1 | Ethanone, 1-[2,3,6,7-tetrahydro-8-hydroxy-10-(2-naphthalenyloxy)-1H,5H-benzo[ij]quinolizin-9-yl]- |
| | (9Cl) |
| 136878-39-2 | Ethanone, 1-[2,3,6,7-tetrahydro-8-hydroxy-10-(2-phenylethyl)-1H,5H-benzo[ij]quinolizin-9-yl]-(9Cl) |
| 151199-70-1 | Spiro[isobenzofuran-1(3H),9′-[1H,5H,9H]xantheno[2,3,4-ij]quinolizine]-5-carboxylic acid, 2′,3′,6′,7′- |
| | tetrahydro-12′-hydroxy-3-oxo-(9Cl) |
| 157649-23-5 | 1H,5H-Benzo[ij]quinolizin-5-one, 9-(3-chloro-1-oxopropyl)-2,3,6,7-tetrahydro-(9Cl) |
| 171205-09-7 | 1H,5H,11H-[1]Benzopyrano[6,7,8-ij]quinolizin-11-one, 2,3,6,7-tetrahydro-9-methoxy-(9Cl) |
| 183736-71-2 | Benzoic acid, 5-nitro-2-[(2,3,6,7-tetrahydro-8-hydroxy-1H,5H-benzo[ij]quinolizin-9-yl)carbonyl]- |
| | (9Cl) |
| 183736-72-3 | Benzoic acid, 4-nitro-2-[(2,3,6,7-tetrahydro-8-hydroxy-1H,5H-benzo[ij]quinolizin-9-yl)carbonyl]- |
| | (9Cl) |
| 195601-29-7 | 4,6(1H,5H)-Pyrimidinedione, 1,3-diethyldihydro-5-[2-(2,3,6,7-tetrahydro-1H,5H-benzo[ij]quinolizin- |
| | 9-yl)-4H-1-benzopyran-4-ylidene]-2-thioxo-(9Cl) |
| 195602-50-7 | Propanedinitrile, [2-(2,3,6,7-tetrahydro-1H,5H-benzo[ij]quinolizin-9-yl)-4H-1-benzopyran-4-ylidene]- |
| | (9Cl) |
| 213389-14-1 | 1H,5H-Benzo[ij]quinolizin-5-one, 7-(3-chlorophenyl)-9-[(4-chlorophenyl)hydroxy(1-methyl-1H- |
| | imidazol-5-yl)methyl]-2,3,6,7-tetrahydro-(9Cl) |
| 213389-15-2 | 1H,5H-Benzo[ij]quinolizin-5-one, 7-(3-chlorophenyl)-9-[(4-chlorophenyl)hydroxy(1-methyl-1H- |
| | imidazol-5-yl)methyl]-2,3,6,7-tetrahydro-, ethanediote (1:1) (salt) (9Cl) |
| 213389-57-2 | 1H,5H-Benzo[ij]quinolizin-5-one, 7-(3-chlorophenyl)-9-[(4-chlorophenyl)hydroxymethyl]-2,3,6,7- |
| | tetrahydro-(9Cl) |
| 213481-01-7 | 1H,5H,11H-[1]Benzopyrano[6,7,8-ij]quinolizin-11-one, 2,3,6,7-tetrahydro-9-hydroxy-(9Cl) |
| 213481-04-0 | Methanesulfonic acid, trifluoro-, 2,3,6,7-tetrahydro-11-oxo-1H,5H,11H-[1]benzopyrano[6,7,8- |
| | ij]quinolizin-9-yl ester (9Cl) |
| 287932-73-4 | 1H,5H-Benzo[ij]quinolizine-9-methanol, ?-ethynyl-2,3,6,7-tetrahydro-?-[4-(2-pyridinyl)phenyl]-(9Cl) |
| 287937-21-7 | 1H,5H-Benzo[ij]quinolizine-9-methanol, ?-ethynyl-2,3,6,7-tetrahydro-?-phenyl-(9Cl) |
| 287937-23-9 | 1H,5H-Benzo[ij]quinolizine-9-methanol, ?-ethynyl-2,3,6,7-tetrahydro-?-[4-(4-morpholinyl)phenyl]- |
| | (9Cl) |
| 287975-51-3 | 1H,5H-Benzo[ij]quinolizine-9-methanol, ?-ethynyl-2,3,6,7-tetrahydro-?-[(4-methoxyphenyl)ethynyl]- |
| | (9Cl) |
| 294876-99-6 | 1H,5H-Benzo[ij]quinolizine-9-carboxylic acid, 2,3,6,7-tetrahydro-1,1,7,7-tetraphenyl-, 2-(4- |
| | nitrophenyl-2-oxoethyl ester (9Cl) |
| 311324-17-1 | 1H,5H-Benzo[ij]quinolizine-9-carboxylic acid, 2,3,6,7-tetrahydro-1,1,7,7-tetraphenyl-(9Cl) |
| 312733-60-1 | 1H,5H-Naphtho[1,2,3-ij]quinolizin-9-ol, 2,3,6,7-tetrahydro-3,3-dimethyl-(9Cl) |
| 313047-94-8 | 1H,5H-Benzo[ij]quinolizine-9-carboxylic acid, 2,3,6,7-tetrahydro-8-hydroxy-(9Cl) |
| 325464-90-2 | 1H,5H-Benzo[ij]quinolizine-9-acetic acid, 2,3,6,7-tetrahydro-?-hydroxy-, ethyl ester, (+)-(9Cl) |
| 326802-00-0 | Benzoic acid, 2-[(2,3-dihydro-10-hydroxy-5,5,7-trimethyl-1H,5H-benzo[ij]quinolizin-9-yl)carbonyl]- |
| | 3,4,5,6-tetrafluoro-(9Cl) |
| 32987-53-4 | Methanone, [4-(dimethylamino)-2-methylphenyl](2,3,6,7-tetrahydro-1H,5H-benzo[ij]quinolizin-9-yl)- |
| | (9Cl) |
| 331254-49-0 | 1H,5H,11H-[1]Benzopyrano[6,7,8-ij]quinolizine-10-carboxaldehyde, 9-ethoxy-2,3,6,7-tetrahydro-11- |
| | oxo-(9Cl) |
| 331648-41-0 | 1H,5H,11H-[1]Benzopyrano[6,7,8-ij]quinolizin-11-one, 2,3,6,7-tetrahydro-9-hydroxy-10- |
| | [[(phenylmethyl)imino]methyl]-(9Cl) |
| 331648-42-1 | 2H,5H,7H,11H-Pyrano[3′,2′:3,4][1]benzopyrano[6,7,8-ij]quinolizine-2,5-dione, 4-chloro-8,9,12,13- |
| | tetrahydro-3-(phenylmethyl)-(9Cl) |
| 33229-60-6 | 1H,5H-Benzo[ij]quinolizine-9-methanol, ?-[p-(dimethylamino)phenyl]-2,3,6,7-tetrahydro-(8Cl) |
| 33229-61-7 | 1H,5H-Benzo[ij]quinolizine-9-methanol, ?-[p-(dimethylamino)phenyl]-2,3,6,7-tetrahydro-?-phenyl- |
| | (8Cl) |
| 33229-62-8 | 1H,5H-Benzo[ij]quinolizine-9-methanol, ?-[4-(dimethylamino)-m-tolyl]-2,3,6,7-tetrahydro-(8Cl) |
| 33229-63-9 | 1H,5H-Benzo[ij]quinolizine-9-methanol, ?-[4-(dimethylamino)-m-tolyl]-2,3,6,7-tetrahydro-?-phenyl- |
| | (8Cl) |
| 33229-65-1 | 1H,5H-Benzo[ij]quinolizine-9-methanol, ?-[4-(dimethylamino)-o-tolyl]-2,3,6,7-tetrahydro-?-phenyl- |
| | (8Cl) |
| 33229-66-2 | Ketone, 4-(dimethylamino)-3,5-xylyl 2,3,6,7-tetrahydro-1H,5H-benzo[ij]quinolizin-9-yl (8Cl) |
| 33229-67-3 | 1H,5H-Benzo[ij]quinolizine-9-methanol, ?-[4-(dimethylamino)-3,5-xylyl]-2,3,6,7-tetrahydro-(8Cl) |
| 33229-68-4 | 1H,5H-Benzo[ij]quinolizine-9-methanol, ?-[4-(dimethylamino)-3,5-xylyl]-2,3,6,7-tetrahydro-?-phenyl- |
| | (8Cl) |
| 344363-85-5 | 1-Propanone, 2,3-dihydroxy-1-(2-hydroxyphenyl)-3-(2,3,6,7-tetrahydro-1H,5H-benzo[ij]quinolizin-9- |
| | yl)-(9Cl) |
| 344363-86-6 | 4H-1-Benzopyran-4-one, 3-hydroxy-2-(2,3,6,7-tetrahydro-1H,5H-benzo[ij]quinolizin-9-yl)-(9Cl) |
| 350492-85-2 | 1H,5H-Benzo[ij]quinolizine-9-carboxylic acid, 2,3,6,7-tetrahydro-1,1,7,7-tetraphenyl-, 2-[1, 1′- |
| | biphenyl]-4-yl-2-oxoethyl ester (9Cl) |
| 350509-47-6 | 1H,5H-Benzo[ij]quinolizine-9-carboxylic acid, 2,3,6,7-tetrahydro-1,1,7,7-tetraphenyl-, 2-(4- |
| | bromophenyl)-2-oxoethyl ester (9Cl) |
| 356062-43-6 | 1H,5H-Benzo[ij]quinolizine-9-methanol, ?-ethynyl-2,3,6,7-tetrahydro-1,1,7,7-tetramethyl-?-phenyl- |
| | (9Cl) |
| 405168-04-9 | Benzoic acid, 2-[[[2-oxo-2-(2,3,6,7-tetrahydro-1H,5H-benzo[ij]quinolizin-9-yl)ethyl]amino]carbonyl]- |
| | (9Cl) |
| 405168-05-0 | Ethanone, 2-amino-1-(2,3,6,7-tetrahydro-1H,5H-benzo[ij]quinolizin-9-yl)-(9Cl) |
| 412031-31-3 | 1,3-Benzenedicarboxylic acid, 2,5-dichloro-4-[(2,3-dihydro-10-hydroxy-5,5,7-trimethyl-1H,5H- |
| | benzo[ij]quinolizin-9-yl)carbonyl]-, 1-(1-methylethyl) ester (9Cl) |
| 49831-47-2 | 1(3H)-lsobenzofuranone, 3-(1,2-dimethyl-1H-indol-3-yl)-3-(2,3,6,7-tetrahydro-1H,5H- |
| | benzo[ij]quinolizin-9-yl)-(9Cl) |
| 54709-85-2 | Furo[3,4-b]pyridin-7(5H)-one, 5-(1,2-dimethyl-1H-indol-3-yl)-5-(2,3,6,7-tetrahydro-1H,5H- |
| | benzo[ij]quinolizin-9-yl)-(9Cl) |
| 57280-49-6 | 1H,5H-Pyrido[3,2,1-gh][1,7]phenanthroline-3,7,9(2H,6H,10H)-trione, 11,12-dihydro-(9Cl) |
| 57323-35-0 | 1H,5H-Pyrido[3,2,1-gh][1,7]phenanthroline-1,7,9(6H,10H)-trione, 2,3,11,12-tetrahydro-(9Cl) |
| 62242-67-5 | 1H-Indeno[4,5,6-ij]quinolizine-1,9(5H)-dione, 2,3,6,7,10,11-hexahydro-2,10-dimethyl-(9Cl) |
| 62242-68-6 | 1H-Indeno[4,5,6-ij]quinolizine-1,9(5H)-dione, 2,3,6,7,10,11-hexahydro-2,10-dimethyl-, mono[(2,4- |
| | dinitrophenyl)hydrazone](9Cl) |
| 62242-69-7 | 1H-Indeno[4,5,6-ij]quinolizine-1,9(5H)-dione, 2,3,6,7,10,11-hexahydro-3,11-dimethyl-(9Cl) |
| 62242-70-0 | 1H-Indeno[4,5,6-ij]quinolizine-1,9(5H)-dione, 2,3,6,7,10,11-hexahydro-3,11-dimethyl-, mono[(2,4- |
| | dinitrophenyl)hydrazone] (9Cl) |
| 62242-71-1 | 1H-Indeno[4,5,6-ij]quinolizine-1,9(5H)-dione, 2,3,6,7,10,11-hexahydro-(9Cl) |
| 62242-72-2 | 1H-Indeno[4,5,6-ij]quinolizine-1,9(5H)-dione, 2,3,6,7,10,11-hexahydro-, mono[(2,4- |
| | dinitrophenyl)hydrazone](9Cl) |
| 62633-19-6 | 1(3H)-Isobenzofuranone, 3-(2,3,6,7-tetrahydro-1H,5H-benzo[ij]quinolizin-9-yl)-(9Cl) |
| 65797-61-7 | 1H,5H-Benzo[ij]quinolizin-5-one, 2,3-dihydro-3,7-dimethyl-9-[2,2,2-trifluoro-1-hydroxy-1- |
| | (trifluoromethyl)ethyl]-(9Cl) |
| 65797-62-8 | 1H,5H-Benzo[ij]quinolizin-5-one, 2,3-dihydro-1,7-dimethyl-9-[2,2,2-trifluoro-1-hydroxy-1- |
| | (trifluoromethyl)ethyl]-(9Cl) |
| 65797-64-0 | 1H,5H-Benzo[ij]quinolizin-5-one, 7-ethyl-2,3-dihydro-9-[2,2,2-trifluoro-1-hydroxy-1- |
| | (trifluoromethyl)ethyl]-(9Cl) |
| 65797-65-1 | 1H,5H-Benzo[ij]quinolizin-5-one, 6-chloro-2,3-dihydro-7-methyl-9-[2,2,2-trifluoro-1-hydroxy-1- |
| | (trifluoromethyl)ethyl]-(9Cl) |
| 65797-66-2 | 1H,5H-Benzo[ij]quinolizin-5-one, 6-bromo-2,3-dihydro-7-methyl-9-[2,2,2-trifluoro-1-hydroxy-1- |
| | (trifluoromethyl)ethyl]-(9Cl) |
| 65828-89-9 | 1H,5H-Benzo[ij]quinolizin-5-one, 2,3-dihydro-7-methyl-9-[2,2,2-trifluoro-1-hydroxy-1- |
| | (trifluoromethyl)ethyl]-(9Cl) |

Fatty acid Analog Compounds

In another aspect of the present invention, fatty acid analog compounds useful in the permanent beautification of mammalian skin are disclosed. Said compounds may be employed individually or in combination with other Beta-ionol and fatty acid analogs to provide numerous beautification benefits to the mammalian skin, while discouraging the onset of skin disorders. To reiterate, the fatty acid analog compounds disclosed herein, like their beta-ionol analog counterparts, serve the fundamental goal of encouraging skin differentiation while discouraging excess skin proliferation. Without wishing to be bound by theory, the fatty acid analog compounds disclosed herein function by entering the cells and engaging in complex interaction with one or more nuclear hormone receptors or their ancillary proteins. This complex interaction leads to the modification of relative rates of production, and thus, the ratios of mRNA in the cells. Such modification ultimately results in the formation of more “productive” cells, and thus, an increased skin matrix per square centimeter of skin—thereby providing a youthful and healthy appearance to treated skin.

The fatty acid analog compounds of the present invention may be illustrated by the following, general structure:

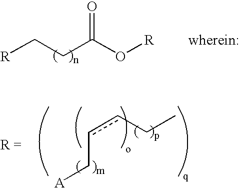

wherein “A” is selected from hydrogen, methyl group, ethyl group and mixtures thereof; further wherein “n,” “o,” “p,” and “m” are variables comprising a value of from about 0 to about 8; further wherein methylene is saturated or unsaturated, substituted or unsubstituted, and/or a constituent of a ring structure, including heterocyclic rings.

In another aspect of the present invention, the optical isomers, diastereomers and enantiomers of the above-depicted formula, as well as pharmaceutically acceptable salts, biohydrolyzable amides, esters, and imides thereof are disclosed and claimed. Said compounds, too, exhibit enhanced beautification and improvement benefits upon application to mammalian, and particularly human, skin. In yet another aspect of the present invention, particularly desired fatty acid analog compounds for use herein are those that are characterized by decreased saturation. Without wishing to be bound by theory, the ability of said compounds to restrict their rotation serves the fundamental goal of increasing the proportion of molecules in the active conformation, thereby reducing the entropy of the subject molecule, and thus, making the molecule more effective against the catalysts of aging mammalian skin.

Representative Examples of Suitable Fatty-Acid Analog Compounds

Indeed, there exists a plethora of suitable fatty-acid analog compounds that may be described by the above-depicted general formula. The below-discussed compounds are intended to serve only as representative structures of the preferred fatty-acid analog compounds for use in the present invention. Other compounds that constitute obvious variations from the above-depicted general formula or the below-listed representative compounds, are intended to be encompassed by the present invention.

In one aspect of the present invention, dihomolinolenic acid, alpha-linolenic acid and similar compounds thereof represent particularly preferred fatty acid analog compounds for use in the present invention. In another aspect of the present invention, gamma linolenic acid, conjugated linolenic acid, arachidonic acid, conjugated linoleic acid, dihomo-gamma-linolenyl-ethanolamide, docosahexanenoic acid; docosapentaenoic acid, docosatetraenoic acid, docosatrienoic acid, linolaidic acid, stereodonic acid and obvious variations thereof constitute suitable fatty acid analog compounds of the present invention. In yet another aspect of the present invention, docosenoic acid, oleic acid, steric acid, elaidic acid, myrstic acid, phytanic acid and obvious variations thereof are employed as suitable fatty acid analog compounds for use herein. To reiterate, the present invention seeks to encompass other compounds that constitute obvious variations from those discussed herein, as suitable fatty acid analog compounds of the present invention.

Beta-Ionol Analog and Fatty Acid Analog Combinations

In yet another aspect of the present invention, the beta-ionol analog and fatty acid analog compounds disclosed herein are employed in combination to beautify and improve the condition of the mammalian skin to which they are applied. Indeed, the precise analog compounds and amounts in which they are combined will depend upon the specific needs and/or abilities of those who seek to practice the present invention. Nevertheless, it has been surprisingly discovered that the ratio of the amount of material of the fatty-acid class to the beta-ionol class in molar terms should parallel their individual ability to induce proliferation, and, in any event, not be less than about 1:100 nor greater than about 100:1 on a molar basis, for optimum activity. The synergy achieved via the combined employment of the beta-ionol and fatty acid analog compounds of the present invention serves not only to beautify and improve mammalian skin, but further to convey anti-cancer benefits.

It should be noted and underscored that it is a fundamental goal of the present invention to disclose various combinations of the present beta-ionol analog and fatty acid analog compounds. In one aspect of the present invention, two or more compounds from the beta-ionol analog class, as disclosed herein, are employed in combination, to achieve various mammalian beautification and anti-cancer benefits. In another aspect of the present invention, two or more compounds of the fatty acid analog class, as disclosed herein, are employed in combination- to beautify mammalian skin and discourage the onset of skin disorders. Indeed, a few such combinations have surprisingly demonstrated immense synergy in the treatment of mammalian skin. The below-listed combinations are intended to serve only as a representative facet of the present invention. It is envisioned that other combinations of the present compounds will also demonstrate synergy in the beautification of mammalian skin, as well as anti-cancer therapy.

TABLE VI
Representative Combinations of Beta-Ionol and Fatty
Acid Analog Compounds

| Composition | Component A | Component B |
|---|---|---|
| Alpha-1 | 0.1-20% dihomolinolenic acid | 0.1-3% bexarotene |
| Beta-1 | 0.1-20% alpha-linolenic acid | 0.1-3% bexarotene |
| Gamma-1 | 0.1-20% gamma-linolenic acid | 0.01-3% bexarotene |
| Delta-1 | 0.1-20% linoleic acid | 0.01-3% bexarotene |
| Alpha-2 | 0.1-20% dihomolinolenic | 0.01-5% melafleur |
| | acid | |
| Beta-2 | 0.1-20% alpha-linolenic | 0.01-5% melafleur |
| | acid | |
| Gamma-2 | 0.1-20% gamma-linolenic | 0.01-5% melafleur |
| | acid | |
| Delta-2 | 0.1-20% linoleic acid | 0.01-5% melafleur |
| Alpha-3 | 0.1-20% dihomolinolenic | 0.01-5% melafleur |
| | acid | alcohol |
| Beta-3 | 0.1-20% alpha-linolenic | 0.01-5% melafleur |
| | acid | alcohol |
| Gamma-3 | 0.1-20% gamma-linolenic | 0.01-5% melafleur |
| | acid | alcohol |
| Delta-3 | 0.1-20% linoleic acid | 0.01-5% melafleur |
| | | alcohol |

Second Aspect—Products and Formulations Incorporating the Present Compounds

The present invention further relates to products and formulations that comprise the beta-ionol analog and fatty acid analog compounds of the present invention, as well as combinations of such products and formulations. Indeed, the combined and systematic use of products and formulations containing the compounds of the present invention serves beautify and improve the condition of the mammalian skin, and particularly human skin, to which they are applied. In accordance with this aspect of the present invention, said products and formulations will take a variety of shapes and forms, depending on the specific needs and/or abilities of the practitioner of the present invention, as well as the purpose for which their employment is sought. In any instance, use of the compounds and products incorporating same in accordance with the present invention results a marked reduction to fine lines and wrinkles, as well as an improvement in the overall appearance of mammalian, and particularly human, skin.

Moreover, the amount of beta-ionol and/or fatty acid analog incorporated into the products and formulations of the present invention will depend on the purpose for which employment of the subject product and/or formulation is desired. Nevertheless, in one aspect of the present invention, the products and formulations disclosed herein will comprise from about 0.0001% to about 10%, preferably from 0.05% to about 10%, more preferably from about 0.1% to about 5%, most preferably from about 0.5% to about 3% of a beta-ionol analog compound. In another aspect of the present invention, the products and formulations disclosed herein will comprise from about 0.5% to about 20%, preferably from about 1% to about 10%, more preferably from about 3% to about 5% of a fatty acid analog compound. In yet another aspect of the present invention, the products and formulations disclosed herein will comprise a combination of both a fatty acid analog compound and a beta-ionol analog compound. In such an instance, the present products and formulations comprise from about 0.05% to 20%, preferably from about 0.1% to about 3%, of a fatty acid analog compound and from about 0.01% to about 5%, preferably from about 0.05% to about 1%, of a beta-ionol analog compound.

Personal Care Products

Thus, in accordance with a first aspect of the present invention, personal care products comprising the beta-ionol analog and fatty acid analog compounds of the present invention are disclosed. Suitable but non-limiting product forms include emulsions, gels, lotions, creams, ointments, mousses, sprays, mists, sticks, powders and combinations thereof. Suitable personal care products comprising the present compounds, include, but certainly are not limited to: hand soaps, hand sanitizers, body washes, mouth washes, toothpastes, shower gels, shampoos, hair conditioners, hand and/or body lotions, facial lotions, facial creams, foundations, lip sticks, rouges, deodorants and combinations thereof. In yet another aspect of the present invention, the personal care products disclosed herein take the form of a wipe product, particularly suitable for wiping or drying a portion of mammalian skin. In such instance, the beta-ionol analogs and fatty acid analog compounds of the present invention are preferably embedded or impregnated into said wipe product. In yet still another aspect of the present invention, the personal care product disclosed herein takes the form of a tissue or towel, also suitable for wiping or drying a portion of mammalian skin. In another aspect of the present invention, the personal care product takes the form of a first aid antiseptic for irritated, injured, or acne-affected skin and/or for pre or post surgical use. In another aspect of the present invention, the personal care product takes the form of a bandage, pad, mask or patch (occlusive, semi-occlusive, or non-occlusive). In yet another aspect of the present invention, the personal care product takes the form of a diaper. Particularly preferred diapers for use in conjunction with the compounds of the present invention are those marketed by The Procter and Gamble Company of Cincinnati, Ohio.

Household Care Products

In another aspect of the present invention, the compounds of the present invention are incorporated into one or more household care products. Indeed, suitable household care products for purposes of the present invention include, but are not limited to: hard surface cleaners, deodorizers, fabric care compositions, fabric cleaning compositions, manual dish detergents, automatic dish detergents, floor care compositions, kitchen cleaners or disinfectants, bathroom cleaners or disinfectants and combinations thereof. In another aspect of the present invention, the household care product takes the form of a wipe or towel, suitable for household cleaning and/or care. In yet another aspect of the present invention, the household care products disclosed herein comprise certain adjunct ingredients. Said adjuncts include, but certainly are not limited to: detersive enzymes, builders, bleaching agents, bleach activators, transitional metal bleach catalysts, oxygen transfer agents and precursors, soil release agents, clay soil removal and/or anti-redeposition agents, polymeric dispersing agents, brightener, polymeric dye transfer inhibiting agents, chelating agents, anti-foam agents, alkoxylated polycarboxylates, fabric softeners, perfumes, carriers, hydrotropes, processing aids, dyes or pigments, solvents for liquid formulations, solid fillers, detersive surfactants and combinations thereof.

Skin Care Products

In a particularly preferred aspect of the present invention, the beta-ionol analog and fatty acid analog compounds of the present invention are incorporated, both alone and in combination (as discussed supra), into a skin care product. In one aspect of the present invention, the skin care product incorporates a dermatologically acceptable carrier to facilitate safe transfer of the present compounds to a desired area of mammalian skin. In another aspect of the present invention, the skin care product of the present invention comprises certain adjunct ingredients. Said adjuncts include, but certainly are not limited to: antimicrobial and antifungal actives, surfactants, desquamation actives, anti-acne actives, anti-wrinkle actives, anti-atrophy actives, anti-oxidants, radical scavengers, chelators, flavonoids, anti-inflammatory agents, anti-cellulite agents, topical anesthetics, tanning actives, sunscreen actives, conditioning agents, thickening agents, detackifying agents, odor control agents, skin sensates, antiperspirants and mixtures thereof. Indeed, a complete description and examples of each of the aforementioned adjunct ingredients is set forth in U.S. Pat. No. 6,294,186, assigned to The Procter and Gamble Company, Cincinnati, Ohio and incorporated herein by reference.

Indeed, in another aspect of the present invention, the compounds disclosed herein are incorporated into a woven or non-woven wipe form of a skin care product, particularly such a product marketed by The Procter and Gamble Company of Cincinnati, Ohio. In yet another aspect of the present invention, the compounds disclosed herein are incorporated into a topical skin care product, including but not limited to, lotions, creams, gels and ointments, particularly those marketed by The Procter and Gamble Company of Cincinnati, Ohio. Indeed, the precise form of suitable skin care products for use in combination with the differentiation inducing compounds of the present invention will depend on the needs and abilities of the formulator of said products.

To reiterate, in one aspect of the present invention, topical compositions comprising the compounds disclosed herein further contain a dermatologically acceptable carrier. The phrase “dermatologically-acceptable carrier”, as used herein, is intended to mean that the carrier is suitable for topical application to the keratinous tissue, has good aesthetic properties, is compatible with the actives of the present invention and any other components, and will not cause any untoward safety or toxicity concerns. A safe and effective amount of carrier is from about 50% to about 99.99%, preferably from about 80% to about 99.9%, more preferably from about 90% to about 98%, and even more preferably from about 90% to about 95% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. Emulsions according to the present invention generally contain a solution as described above and a lipid or oil. Lipids and oils may be derived from animals, plants, or petroleum and may be natural or synthetic (i.e., man-made). Preferred emulsions further contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986). The emulsion may also contain an anti-foaming agent to minimize foaming upon application to the keratinous tissue. Anti-foaming agents include high molecular weight silicones and other materials well known in the art for such use.

Skin Care Actives

The compositions of the present invention may optionally contain one or more additional skin care actives or combination of skin care actives. The skin care active may be included as a substantially pure material, or as an extract obtained by suitable physical and/or chemical isolation from natural (e.g., plant) sources. In a preferred embodiment, where the composition is to be in contact with human keratinous tissue, the additional skin care active(s) should be suitable for application to keratinous tissue, that is, when incorporated into the composition they are suitable for use in contact with human keratinous tissue without undue toxicity, incompatibility, instability, allergic response, and the like within the scope of sound medical judgment. The CTFA Cosmetic Ingredient Handbook, Second Edition (1992) describes a wide variety of cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

Anti-Wrinkle Actives/Anti-Atrophy Actives

The compositions of the present invention may contain a safe and effective amount of one or more anti-wrinkle actives or anti-atrophy actives. Exemplary anti-wrinkle/anti-atrophy actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as lactic acid and glycolic acid or beta-hydroxy acids such as salicylic acid and salicylic acid derivatives such as the octanoyl derivative), phytic acid, lipoic acid; lysophosphatidic acid, and skin peel agents (e.g., phenol and the like), which enhance the keratinous tissue appearance benefits of the present invention, especially in regulating keratinous tissue condition, e.g., skin condition.

Anti-Oxidants/Radical Scavengers

The compositions of the present invention may include a safe and effective amount of an anti-oxidant/radical scavenger, preferably from about 0.1% to about 10%, more preferably from about 1% to about 5%, of the composition. The anti-oxidant/radical scavenger is especially useful for providing protection against UV radiation which can cause increased scaling or texture changes in the stratum corneum and against other environmental agents which can cause skin damage. Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used. Preferred anti-oxidants/radical scavengers are selected from tocopherol acetate, other esters of tocopherol, and mixtures thereof. Tocopherol acetate is especially preferred.

Chelators

The compositions of the present invention may contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. Preferred chelators useful in compositions of the subject invention are furildioxime, furilmonoxime, and derivatives thereof.

Flavonoids

The compositions of the present invention may contain a safe and effective amount of flavonoid compound. Flavonoids are broadly disclosed in U.S. Pat. Nos. 5,686,082 and 5,686,367, both of which are herein incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents.

Anti-Inflammatory Agents

A safe and effective amount of an anti-inflammatory agent may be added to the compositions of the present invention, preferably from about 0.1% to about 10%, more preferably from about 0.5% to about 5%, of the composition. Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used.

A second class of anti-inflammatory agents that is useful in the compositions includes the nonsteroidal anti-inflammatory agents. The variety of compounds encompassed by this group are well-known to those skilled in the art. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974). Mixtures of these non-steroidal anti-inflammatory agents may also be employed, as well as the dermatologically acceptable salts and esters of these agents.

Finally, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄.

Anti-Cellulite Agents

The compositions of the present invention may contain a safe and effective amount of an anti-cellulite agent. Suitable agents may include, but are not limited to, xanthine compounds (e.g., caffeine, theophylline, theobromine, and aminophylline).

Topical Anesthetics

The compositions of the present invention may contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Tanning Actives

The compositions of the present invention may contain a safe and effective amount of a tanning active, preferably from about 0.1% to about 20% of dihydroxyacetone as an artificial tanning active.

Skin Lightening Agents

The compositions of the present invention may contain a skin lightening agent. When used, the compositions preferably contain from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in the PCT publication No. 95/34280, in the name of Hillebrand, corresponding to PCT Application No. U.S. 95/07432, filed Jun. 12, 1995; and co-pending U.S. application Ser. No. 08/390,152 filed in the names of Kvalnes, Mitchell A. DeLong, Barton J. Bradbury, Curtis B. Motley, and John D. Carter, corresponding to PCT Publication No. 95/23780, published Sep. 8, 1995.

Skin Soothing and Skin Healing Actives

A safe and effective amount of a skin soothing or skin healing active may be added to the present composition, preferably, from about 0.1% to about 30%, more preferably from about 0.5% to about 20%, still more preferably from about 0.5% to about 10%, by weight of the composition formed. Skin soothing or skin healing actives suitable for use herein include panthenoic acid derivatives (including panthenol, dexpanthenol, ethyl panthenol), aloe vera, allantoin, bisabolol, and dipotassium glycyrrhizinate.

Antimicrobial and Antifungal Actives

The compositions of the present invention may contain an antimicrobial or antifungal active. A safe and effective amount of an antimicrobial or antifungal active may be added to the present compositions, preferably, from about 0.001% to about 10%, more preferably from about 0.01% to about 5%, and still more preferably from about 0.05% to about 2%. Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Sunscreen Actives

Exposure to ultraviolet light can result in excessive scaling and texture changes of the stratum corneum. Therefore, the compositions of the subject invention may contain a safe and effective amount of a sunscreen active. As used herein, “sunscreen active” includes both sunscreen agents and physical sunblocks. Suitable sunscreen actives may be organic or inorganic. Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition. A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

Also particularly useful in the compositions are sunscreen actives such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range. A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Particulate Material

The compositions of the present invention may contain a safe and effective amount of a particulate material, preferably a metallic oxide. These particulates can be coated or uncoated, charged or uncharged. Charged particulate materials are disclosed in U.S. Pat. No. 5,997,887, to Ha, et al., incorporated herein by reference. Particulate materials useful herein include; bismuth oxychloride, iron oxide, mica, mica treated with barium sulfate and TiO2, silica, nylon, polyethylene, talc, styrene, polypropylene, ethylene/acrylic acid copolymer, titanium dioxide, iron oxide, bismuth oxychloride, sericite, aluminum oxide, silicone resin, barium sulfate, calcium carbonate, cellulose acetate, polymethyl methacrylate, and mixtures thereof.

Conditioning Agents

The compositions of the present invention may contain a safe and effective amount of a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to Orr et al, issued Dec. 11, 1990. Also useful are various C₁-C₃₀ monoesters and polyesters of sugars and related materials.

Thickening Agent (Including Thickeners and Gelling Agents)

The compositions of the present invention may contain a safe and effective amount of one or more thickening agents, preferably from about 0.1% to about 5%, more preferably from about 0.1% to about 4%, and still more preferably from about 0.25% to about 3%, by weight of the composition. Suitable classes of thickening agents for use in the present invention, include, but certainly are not limited to, the following: carboxylic acid polymers, crosslinked polyacrylate polymers, polyacrylamide polymers, polysaccharides, gums and combinations thereof.

Topical Formulations Comprising Present Compounds

In yet another highly preferred aspect of the present invention, the beta-ionol analog and fatty acid analog compounds disclosed herein are formulated into compositions for topical application onto mammalian, and particularly human, skin. In another aspect of the present invention the topical formulations disclosed herein include a safe and effective amount of differentiation-inducing agents and other ingredients that are adapted to enhance the appearance of the mammalian skin onto which they are applied.

By “safe and effective amount”, it is intended that an incorporated amount of a compound or composition be high enough to significantly improve the appearance of the skin, but low enough to discourage side effects, which may actually reduce the appearance and beauty of the skin. Indeed, the safe and effective amount of an agent for use in the compounds and/or compositions of the present invention will vary depending on one or more of the following factors: the nature of the skin for which treatment is sought, the age and physical condition of the skin for which treatment is sought, the severity of any existing skin conditions, the intended duration of the treatment, the existence and nature of any concurrent therapy, the particular agent for which employment is sought, the particular excipients utilized, and the needs and/or abilities of the formulator of the present compounds and compositions. Nevertheless, the appropriate amount of the agent, preferably the beta-ionol analog and fatty-acid analog compounds disclosed herein, to be incorporated into the present compositions may be determined by routine experimentation with animal models. Indeed, one such model includes, but certainly is not limited to, intact and aged murine models of mammalian, and particularly human, skin.

The differentiation-promoting compounds of the present invention may be administered systemically, e.g., orally and/or parenterally, including subcutaneous or intravenous injection, and/or intranasally, but especially transdermally. In one aspect of the present invention, the differentiation-promoting compounds disclosed herein are applied directly to the mammalian skin for which treatment is sought in a unit dosage form. As discussed in the “Formulations” section of the present disclosure, the precise amount of the present compounds incorporated into a unit dosage form will depend upon one or more factors disclosed hereinbefore, and particularly the needs and/or abilities of the formulator of the present compositions and the nature of the mammalian skin for which treatment is desired.

In yet another aspect of the present invention, the dose forms for use with the present compounds and compositions include nasal, transdermal, rectal, sublingual, oral and combinations thereof. In another aspect of the present invention, one or more carriers suitable for use in the present invention may be employed to achieve delivery of the present compounds and compositions, and particularly for injection or surgical implants. Said carriers include, but certainly are not limited to: hydrogels, controlled- or sustained release devises, polylactic acid, collagen matrices, and combinations thereof. In another aspect of the present invention, implant devices are coated with the differentiation-promoting compounds and/or formulations disclosed herein. In another aspect of the present invention, the differentiation-promoting compounds and/or formulations disclosed herein are dissolved in a buffer and mixed with a collagen gel for coating onto the porous end of an implant device.

Indeed, in a further aspect of the present invention, the compounds disclosed herein are administered orally. In this respect, oral forms suitable for administration of the present compounds and formulations include, but certainly are not limited to: liposomes, lipid emulsions, proteinaceous cages, other excipients and combinations thereof. Use of the term “excipients” herein is intended to encompass any physiologically inert, pharmacologically inactive material known to those of ordinary skill in the art. Suitable excipients for use in the present invention are compatible with the physical and chemical characteristics of the particular differentiation-promoting ingredient for which employment is sought, as well as the mammalian skin substrate for which application is desired. In one aspect of the present invention, suitable excipients for use herein include, but are not limited to, polymers, resins, plasticizers, fillers, lubricants, binders, disintegrants, solvents, co-solvents, buffer systems, surfactants, preservatives, sweetening agents, flavoring agents, fragrance agents pharmaceutical grade dyes, pigments and combinations thereof.

When the use of a flavoring agent excipient in the compositions of the present invention is desired, suitable such agents may be selected from those described in Remington's Pharmaceutical Sciences, 18th Edition, Mack Publishing Company, 1990, pp. 1288-1300, incorporated by reference herein. Dyes, or pigments suitable for use in the present invention include, but are not limited to, those described in Handbook of Pharmaceutical Excipients, Second Edition pp. 126-134, 1994 by the American Pharmaceutical Association & the Pharmaceutical Press, incorporated by reference herein.

Suitable solvents and co-solvents for use in the present invention include, but are not limited to, water, ethanol, glycerin, propylene glycol, polyethylene glycol and combinations thereof. Suitable buffer systems for use as excepients herein include, but are not limited to potassium acetate, boric carbonic, phosphoric, succinic, malic, tartaric, citric, acetic, benzoic, lactic, glyceric, gluconic, glutaric, glutamic and combinations thereof. In one aspect of the present invention suitable buffer systems for use herein are phosphoric, tartaric, citric, and potassium acetate.

Suitable surfactants for use as excepients in the present invention include, but are not limited to, polyoxyethylene sorbitan fatty acid esters, polyoxyethylene monoalkyl ethers, sucrose monoesters and lanolin esters, ethers and mixtures thereof. Moreover, suitable preservatives for use as excepients of the present invention include, but are not limited to, phenol, alkyl esters of parahydroxybenzoic acid, benzoic acid and the salts thereof, boric acid and the thereof, sorbic acid and the salts thereof, chlorobutanol, benzyl alcohol, thimerosol, phenylmercuric acetate and nitrate, nitromersol, benzalkonium chloride, cetylpyridinium chloride, methyl paraben, and propyl paraben. Particularly preferred are the salts of benzoic acid, cetylpyridinium chloride, methyl paraben, propyl paraben and combinations thereof.

Suitable sweeteners for use with the differentiation-inducing compounds disclosed herein include, but are not limited to, sucrose, glucose, saccharin, aspartame and combinations thereof. In another aspect of the present invention, sucrose, saccharin and combinations thereof are particularly preferred sweeteners for use with the present compounds. Suitable binders for use in conjunction with the present compounds include, but are not limited to methylcellulose, sodium carboxymethylcellulose, hydroxypropylmethylcellulose, carbomer, prodione, acacia, guar gum, xanthan gum, tragcanth and combinations thereof. In yet another aspect of the present invention, particularly preferred binders for use herein include, but are not limited to, methylcellulose, carbomer, xanthan gum, guar gum, povidone, sodium carboxymethylcellulose and combinations thereof.

Suitable fillers for use with the Beta-ionol analog and fatty acid analog compounds disclosed herein include, but are not limited to lactose, sucrose, maltodextrin, mannitol, starch, microcrystalline cellulose and combinations thereof. Suitable plasticizers for use with the present compounds include, but are not limited to polyethylene glycol, propylene glycol, dibutylphthalate, and castor oil, acetylated monoglycerides, triactin and combinations thereof. In another aspect of the present invention, suitable lubricants for use herein include, but are not limited to, magnesium stearate, stearic acid, talc and combinations thereof. Indeed, suitable disintegrants for use with the compounds of the present invention include, but are not limited to, crospovidone, sodium carboxymethyl starch, sodium starch glycollate, sodium carboxymethyl cellulose, alginic acid, clays, ion exchange resins and combinations thereof. Suitable polymers for use as excepients of the present invention, include but are not limited to, hydroxypropylmethylcellulose (HPMC), hydroxypropylcellulose (HPC), carboxymethylcellulose, acrylic resins such as Eudragit® RL30D, manufactured by Rohm Pharma GmbH Weiderstadt, West Germany, methylcellulose, ethylcellulose, polyvinylpyrrolidone, commercially available film-coating preparations such as Dri-Klear, manufactured by Crompton & Knowles Corp., Mahwah, N.J., Opadry manufactured by Colorcon, West Point, Pa. and combinations thereof. It should be reiterated and underscored that the precise ingredients and suitable excepients for use with the compounds of the present invention will depend on several factors, and particularly the needs and/or abilities of the formulator and the nature of the skin for which treatment with the present compounds is desired. Nevertheless, the above discussion is intended only to serve as a guide to a person of ordinary skill in the art. Certainly, compounds analogous or similar to those listed above will also be suitable for employment with the compounds of the present invention.

Articles of Manufacture & Kits

Moreover, articles of manufacture comprising the beta-ionol analogs and fatty acid analog compounds of the present invention and/or one or more of the aforementioned products, are intended for personal care, skin care and household care applications. The article of manufacture of the present invention encompasses one or more products as described hereinbefore that may be packaged in a container or dispenser with a set of instructions for the consumer. The article of manufacture of the present invention typically comprises (a) container or dispenser, (b) product and (c) set of instructions to apply said product to an appropriate substrate to convey actual and permanent beautification and improvement benefits to mammalian skin. Containers and/or dispensers suitable for the article of manufacture of the present invention include, but are not limited to: PET bottles and tubs, flow-wrap pouches, foaming dispensers, spray dispensers and combinations thereof. To reiterate, the article of manufacture of the present invention further comprises a set of instructions in association with the container. By “in association with,” it is meant that the instructions are either directly printed on the container or dispenser itself or presented in a different fashion including, but not limited to: a brochure, print advertisement, electronic advertisement and/or verbal communication, so as to communicate the set of the instructions to a consumer of the article of manufacture.

The set of instructions typically comprise the instructions relating to the use of the product to apply the beta-ionol analog and fatty acid analog compounds of the present invention onto a suitable substrate for which treatment is sought. The set of instructions may further comprise the instruction to allow the present compounds to remain on the treated substrate, without rinsing or otherwise removing the compounds from the treated substrate. Nevertheless, the precise instructions included with the article of manufacture of the present invention will depend on the specific compounds and the product for which the inclusion of instructions is desired and the substrate onto which application of the product is intended. In another aspect of the present invention, the instructions included in the present articles of manufacture coincide with the methods set forth in the “Methods of Using the Present Compounds and Products” section of the present disclosure.

Third Aspect—Methods of Using the Present Compounds and Products

In yet another preferred aspect of the present invention, methods of using the beta-ionol analog and fatty acid analog compounds discussed herein, are disclosed. The compounds of the present invention are useful in providing actual and permanent beautification and improvement benefits to mammalian, and particularly human, skin. Indeed, when practiced in accordance with the present invention, the differentiation-inducing compounds and compositions disclosed herein serve to increase the appeal of the mammalian skin to which they are applied, while maintaining a youthful appearance thereof. Further and compelling, the compounds and products of the present invention discourage the onset of physical ailments resulting from existing skin conditions and prevent irritation to mammalian skin following application of the present compounds. In a fundamental aspect of the present invention, the compounds, compositions and products disclosed herein are useful for employment in cosmetics, creams and oils, and in compositions for the treatment of various skin dysfunctions and cancer.

In one aspect of the present invention, the compounds and/or products disclosed herein are directly applied to the mammalian skin for which treatment is desired. In another aspect of the present invention, the compounds and/or products disclosed herein are applied transdermally to the mammalian skin for which treatment is sought. The exact amount of the differentiation inducing compounds and/or nature of products will depend upon the needs and abilities of the formulator and practitioner of the present methods. In one aspect of the present invention, the compounds of the present invention are conveyed to the mammalian skin for which treatment is desired at least once per day. Once applied, the compositions are rubbed on the treated surfaces for a period of time to ensure coverage. In another aspect of the present invention, transdermal dosages are designed and intended to attain minimal serum or plasma levels, based upon techniques known to those skilled in the art of pharmacokinetics and formulations. The following non-limiting examples serve to further illustrate the use of the agents of the present invention.

The antimicrobial compositions and products of the present invention are suitable for a variety of uses. Indeed, suitable uses of the present compositions include, but certainly are not limited to, the beautification of mammalian skin, the reduction of fine lines and wrinkles of mammalian skin and the treatment of cancer. In one aspect of the present invention, the methods disclosed herein comprise the step of topically applying a composition or product comprising same to mammalian, and particularly human, skin for which treatment is desired. Examples of areas and/or surfaces in need of treatment, against which the compounds and compositions of the present invention are effective, include, but are not limited to: the face, the neck, one or more hands, a nose, a nasal canal or passage, an article of clothing, a hard surface, irritated, acne-affected, or injured skin, pre or post surgical areas and combinations thereof.

### PREPARATIVE EXAMPLES

Preparation of Novel, Beta-Ionol Analog Compounds

Compounds are analyzed using proton and carbon NMR spectroscopy, elemental analyses, mass spectral analyses, high-resolution mass spectral analyses and/or infrared spectral analysis as appropriate. Purification is achieved by recrystallization or pressure chromatography. Thin-layer chromatography is performed on glass silica gel plates and visualized using ultraviolet (UV) light and/or a solution of 5% phosphomolybdic acid in ethanol.

### Example 1

### Preparation of 4-(8,8-Dimethyl-1,2,3,4,5,6,7,8-octahydro-naphthalene-2-carbonyl)-benzoic Acid Methyl Ester (1I)

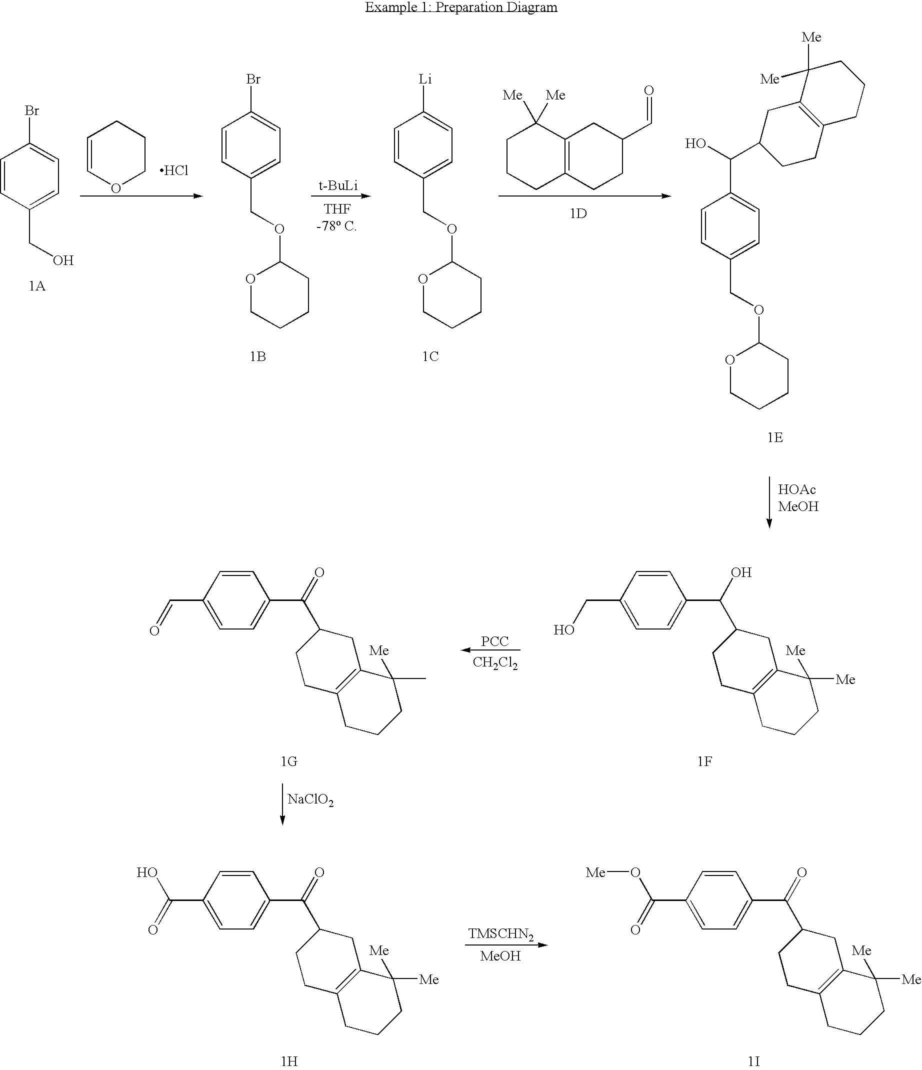

Preparation of 2-(4-Bromo-benzyloxy)-tetrahydropyran (1B):

To a solution of 1A in methylene chloride is added 1.2 equivalents of dihydropyran and a catalytic amount of a 1 M HCl in diethyl ether solution. The material is stirred at room temperature and the reaction is monitored by TLC. After one hour, solid sodium bicarbonate is added, followed by a portion of brine. The layers are separated and the organic layer is concentrated, dried over magnesium sulfate, and purified by flash chromatography to yield a colorless oil, 2-(4-Bromo-benzyloxy)-tetrahydropyran, 1 B.

Lithiation and Addition to Produce 1E:

To a solution of 1B in anhydrous THF is added dropwise at negative 78 degrees C. 2 equivalents of a 1.6 M solution of t-BuLi. This is stirred for one hour, producing in situ, compound 1C, which is not isolated, but used at once in the next step. To this solution is carefully added commercially available melafleur 1D (International Flavors and Fragrances) and the solution allowed to reach room temperature over the course of one hour. One equivalent of 1 M HCl in a saturated aqueous solution of NH₄Cl is then added, along with a portion of 1% hexanes in ethyl acetate. The layers are separated and the organic layer is washed with brine, dried over magnesium sulfate, and concentrated. The crude material is purified by flash chromatography to give the colorless oil, 1E.

Preparation of (8,8-Dimethyl-1,2,3,4,5,6,7,8-octahydro-naphthalen-2-yl)-(4-hydroxymethyl-phenyl)-methanol, 1F:

A solution of 1E in a 1:1 HOAc and MeOH is stirred at room temperature overnight. The next day to the reaction is added a portion of ethyl acetate and sufficient aqueous 1N NaOH to neutralize the acid. The layers are separated and the organic layer is washed with brine, dried over magnesium sulfate, and concentrated. The crude material is purified by flash chromatography to give the colorless oil, 1F.

Preparation of 4-(8,8-Dimethyl-1,2,3,4,5,6,7,8-octahydro-naphthalene-2-carbonyl)-benzaldehyde, 1G:

To a stirred solution of 1F in methylene chloride is added 2.4 equivalents of pyridinium chlorochromate (PCC, Aldrich Chemical Company) and a portion of Celite. The mixture is stirred and the progress of the reaction is monitored by TLC. After two hours and when the reaction is adjudged complete by TLC analysis, the solution is vacuum filtered through a column of Fluorosil, and concentrated to a white solid. The crude solid is further purified by flash chromatography to yield a white solid, 1G.

Preparation of 4-(8,8-Dimethyl-1,2,3,4,5,6,7,8-octahydro-naphthalene-2-carbonyl)-benzoic Acid, 1H:

A stirred solution is prepared of tert-butyl alcohol, water, 2-methyl-2-butene and monosodium phosphate. To this solution is added solid NaClO₂ and the aldehyde 1G. This solution is stirred at room temperature for and the disappearance of the aldehyde is followed by TLC analysis. When the reaction is judged complete, ethyl acetate is added and the reaction mixture is washed thrice with brine. The layers are separated and the organic layer is dried over magnesium sulfate and concentrated. The crude material is purified by flash chromatography (with 1% formic acid being added to the normal chromatography solvent) to give the white solid 4-(8,8-Dimethyl-1,2,3,4,5,6,7,8-octahydro-naphthalene-2-carbonyl)-benzoic acid, 1H.

Preparation of 4-(8,8-Dimethyl-1,2,3,4,5,6,7,8-octahydro-naphthalene-2-carbonyl)-benzoic Acid Methyl Ester, 1I:

To a solution of 1H in methanol is added an excess of a TMS-diazomethane solution (Aldrich Chemical Company). The mixture is stirred for at least two hours and the evolution of nitrogen gas is observed. When the TLC analysis indicates the reaction is complete, the solvent is removed and the crude is purified by flash chromatography to yield 1H.

### Example 2

### Preparation of Monocyclic and Bicyclic Beta-Ionol Analog Core Compounds

The novel, monocyclic and bicyclic core Beta-ionol analog compounds of the present invention are prepared using substantially the same procedures as those described in Example 1, substituting the appropriate starting materials. The skilled artisan may adjust the temperature, pressure, atmosphere, solvents or the order of reaction steps as appropriate. Additionally, the skilled artisan may employ protecting groups to block side reactions or increase yields as appropriate. The skilled artisan might employ a variety of known techniques to isolate largely one enantiomer, or to create an enantiomeric excess of one enantiomer over another. All such modifications can be readily done by the skilled artisan in the art of organic synthesis and thus are within the scope of the invention. A representative list of the novel, monocyclic and bicyclic Beta-ionol analog core compounds produced via the preparation procedure detailed herein is found in Tables I and II of the present disclosure.

### Example 3

### Preparation of E-1-Methoxy-4-[1-methyl-3-(2,6,6-trimethyl-cyclohex-1-enyl)-propenyl]-benzene (3B) and Z-1-Methoxy-4-[1-methyl-3-(2,6,6-trimethyl-cyclohex-1-enyl)-propenyl]-benzene (3C) Beta-Ionol Analog Compounds

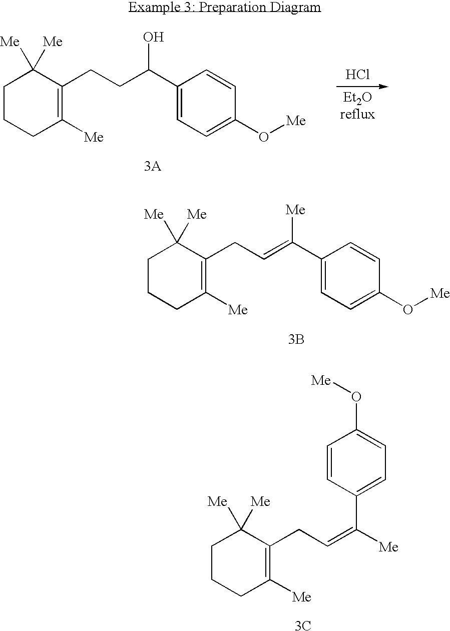

Compounds of Example 3 are synthesized directly from the compounds of Example 1 by heating with hydrochloric acid. Produced are Beta ionol analogs E-1-Methoxy-4-[1-methyl-3-(2,6,6-trimethyl-cyclohex-1-enyl)-propenyl]-benzene (3B) and Z-1-Methoxy-4-[1-methyl-3-(2,6,6-trimethyl-cyclohex-1-enyl)-propenyl]-benzene (3C).

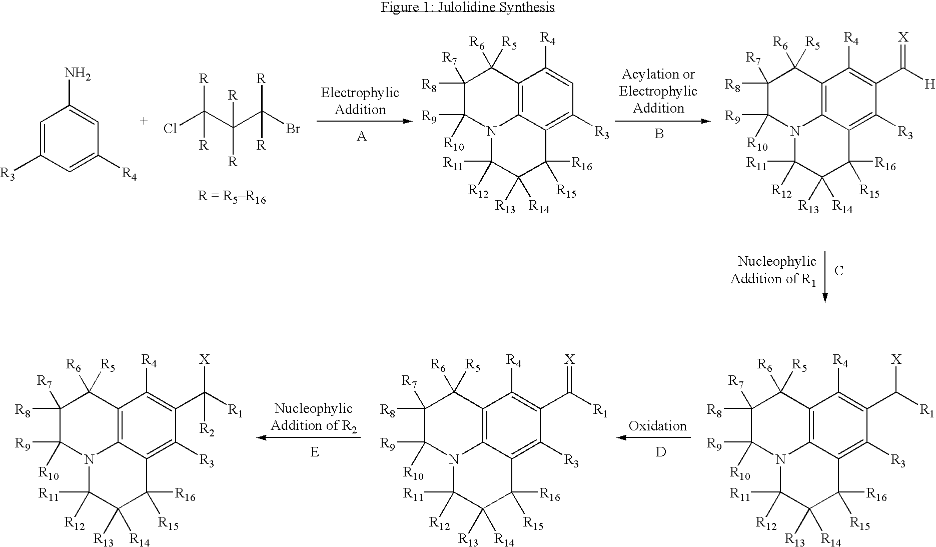

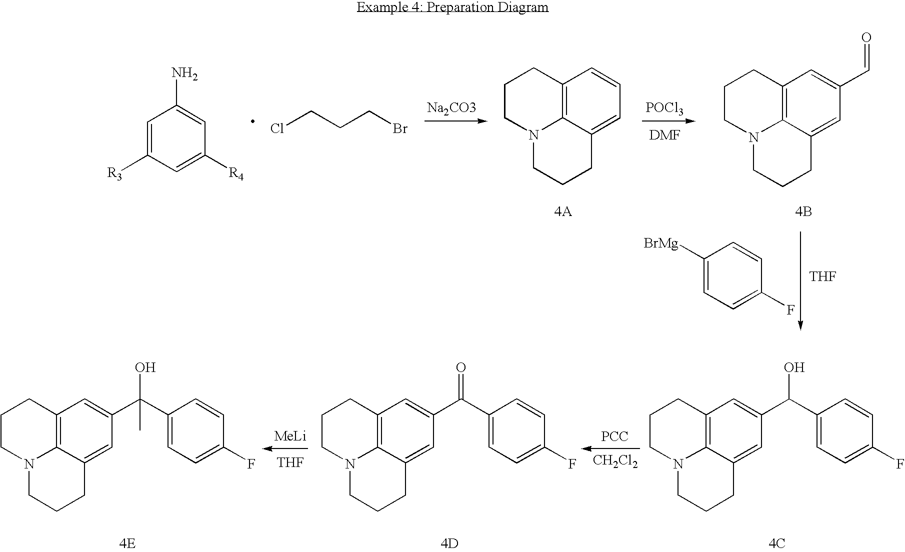

Unless otherwise noted, all reactions were performed under a Nitrogen atmosphere in flame dried flasks. Solvents are all anhydrous and were obtained from Aldrich.

### Example 4A

### Preparation of 2,3,6,7-Tetrahydro-1H,5H-pyrido[3,2,1-ij]quinoline

A 3-neck flask equipped with a thermometer, overhead stirrer, and pressure equalizing funnel is filled with 4A° sieves and fitted with a condensor, is added analine (1 eq), 1-bromo-3-chloropropane (15 eq.), and anhydrous sodium carbonate (4 eq.) is heated to 150° C. for 4 hours. The reaction mixture is poured into water and neutralized with 1N HCl and extracted 3× with CH₂Cl₂, washed with brine, dried over MgSO₄ and concentrated on vacuum. Purification is achieved via silica chromatography.

### Example 4B

### 2,3,6,7-Tetrahydro-1H,5H-pyrido[3,2,1-ij]quinoline-9-carbaldehyde

POCl₃ (1.1 eq) is dissolved in DMF (1M) with a cool water bath and stirred for 15 minutes. A solution of 4A in DMF (1M) is added dropwise and stirred for 30 minutes. The temperature is raised to 100° C. for 1 hr. and then cooled to room temperature. The reaction mixture is poured into water, adjusted to pH=7.0 with saturated Na₂CO₃, and extracted 4× with CH₂Cl₂, rinsed 2× with brine, rinsed 5× with H₂O to remove DMF, dried over MgSO₄ and is concentrated on vacuum. Purification is achieved via silica chromatography.

### Example 4C

### (4-Fluoro-phenyl)-(2,3,6,7-tetrahydro-1H,5H-pyrido[3,2,1-ij]quinolin-9-yl)-methanol

To a solution of 4B (1.1 eq.) in THF (1M) in a flask fitted with a reflux condensor, is added the Julolidine-9-carboxadehyde (1 eq.), dropwise. The reaction is stirred at room temperature for 1 hour. The reaction mixture is poured into 50 mL saturated NH₄Cl with an amount of 1N HCl sufficient to bring the solution to pH=7.0. The solution is extracted 3× with EtOAc (containing 5% Hexane), is rinsed with brine, is dried over MgSO₄ and is concentrated on vacuum. Purification is achieved via silica chromatography.

### Example 4D

### (4-Fluoro-phenyl)-(2,3,6,7-tetrahydro-1H,5H-pyrido[3,2,1-ij]quinolin-9-yl)-methanone

To a solution of Julolidine-9-(4-Fluoro-phenyl)-9-hydroxy (1 eq.) and celite (mass equivalent of PCC) in CH₂Cl₂ (0.2M) is added pyridinium chlorochromate (PCC 1.1 eq.). The mixture is stirred for 4 hours. The reaction mixture is filtered through Fluorosil, is washed with CH₂Cl₂. The solution is extracted 3× with EtOAc (containing 5% Hexane), is rinsed with brine, is dried over MgSO₄ and is concentrated on vacuum. Purification is achieved via silica chromatography.

### Example 4E

### 1-(4-Fluoro-phenyl)-1-(2,3,6,7-tetrahydro-1H,5H-pyrido[3,2,1-ij]quinolin-9-yl)-ethanol

To a solution of methyl lithium (1.1 eq. of a 1.4M solution in ether, (Aldrich Chemical)) in THF (1M), in a flask fitted with a reflux condensor, is added the Julolidine-9-(4-Fluoro-phenyl)-9-ketone (1 eq.), dropwise. The reaction is stirred at room temperature for 1 hour. The reaction mixture is poured into 50 mL saturated NH₄Cl with an amount of 1N HCl sufficient to bring the solution to pH=7.0. The solution is extracted 3× with EtOAc (containing 5% Hexane), washed with brine, dried over MgSO₄ and is concentrated on vacuum. Purification is achieved via silica chromatography.

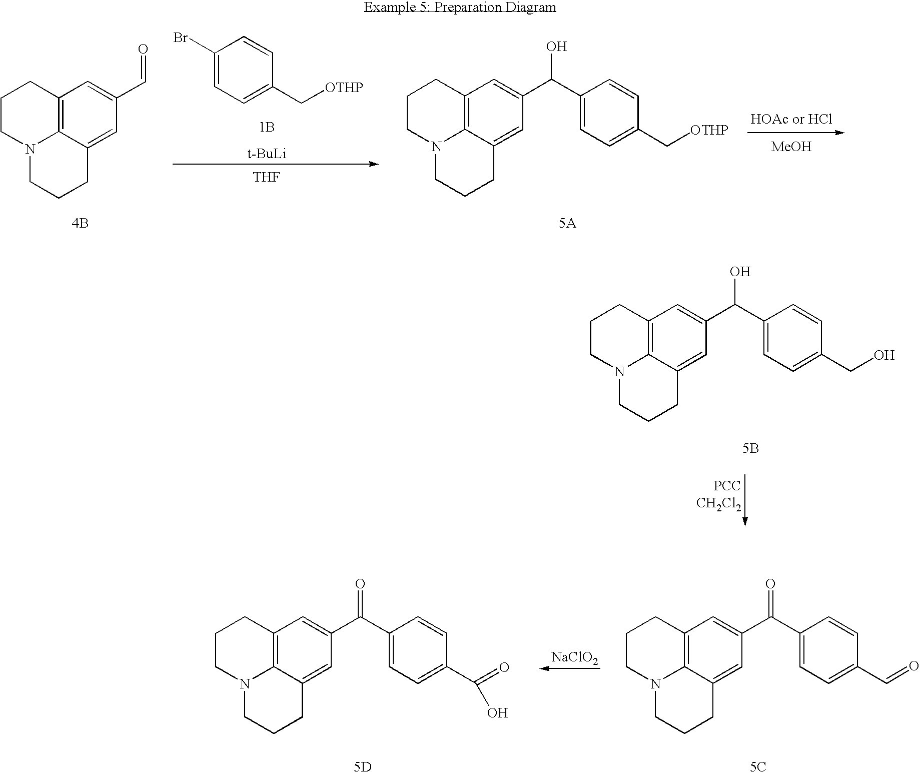

All compounds in Example 5 are synthesized by methods analogous to those used in Example 4.

### Example 6

### Topical Administration of Beta-Ionol Analog Compounds

A Beta-ionol analog, melafleur alcohol, is administered topically to a human directly onto an area of the skin in need of beautifying. After two weeks of daily treatments, the treated areas of the skin exhibit increased health and vitality.

### Example 7

### Preparation of Tablets Containing Beta-Ionol Analog Compounds

Formulations (compositions) in the form of tablets are prepared by conventional methods, such as mixing and direct compaction, formulated as follows

EXAMPLE 7
Preparation Table

| Ingredient | Quantity (mg per tablet) |
|---|---|
| Tetramethylene Beta ionol | 200 |
| Microcystalline Cellulose | 100 |
| Sodium Starch Glycollate | 30 |
| Magnesium Stearate | 3 |

The above tablet, when administered orally once a day, substantially increases the beauty of the mammalian skin onto which it is applied.

### Example 8

### Formulation of Liquid Compositions Comprising Present Compounds

A composition in liquid form is prepared by conventional methods, formulated as follows:

EXAMPLE 8
Preparation Table

| Ingredient | Quantity |
|---|---|
| Beta ionic acid ethyl ester | 500 mg |
| Dihomolinolenic acid | 500 mg |
| Propylene glycol | 5 ml |
| Ethyl alcohol | 5 ml |

1.0 mL of the above composition, when administered once a day, substantially increases the beauty and health of the mammalian skin onto which it is applied.

### Example 9

### Preparation of Skin Care Topical Product Comprising Present Compounds

A skin care, topical product is prepared by formulating the liquid composition of Example 3 into such a product. Indeed, a preferred example of such a product is one stemming from the Oil of Olay topical skin care line, owned and distributed by The Procter and Gamble Company of Cincinnati, Ohio.

### Example 10

### Preparation of Skin Care Wipe Product Comprising Present Compounds

A skin care wipe product is prepared by impregnating such a wipe with the liquid composition of Example 3. Such a wipe may be impregnated by techniques known and readily available to those skilled in the art. Indeed, a preferred example of a wipe product is the Oil of Olay Facial Wipes, owned and distributed by The Procter and Gamble Company of Cincinnati, Ohio.

What is claimed is:

1. A beta-ionol analog compound having the formula: 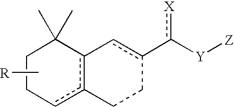
wherein “X” is a single or double bonded moiety comprising from 0 to about 12 substituted or unsubstituted carbon atoms; from 0 to about 2 heteroatoms, selected from substituted, unsubstituted, cycloalkyl and aromatic moieties of NH, S, O and combinations thereof; “Z” is a single, double, or triple bonded moiety containing from 0 to about 12 carbon atoms in a chain, optionally including a cycloalkyl or aromatic ring, both of which may be further substituted; “Y” is (CH₂)ₙ, wherein “n” is a variable having a value of from 0 to about 3; “R” is a group which may be substituted onto any ring if two or more are present and is selected from no greater than three independently selected substituted, unsubstituted, alkyl, cycloalkyl or aromatic moieties including CH₃, CH₂CH₃, NR₁R₂, SR, OR and combinations thereof.

2. A fatty acid analog compound having the formula: 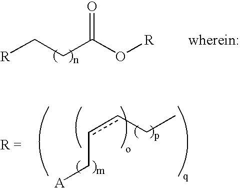
wherein “A” is selected from hydrogen, methyl, ethyl and mixtures thereof; further wherein “n”, “o”, “p”, and “m” are variables comprising a value of from 0 to about 8; further wherein methylene is saturated, unsaturated, substituted, unsubstituted, a constituent of a ring structure and combinations thereof.

3. A mixture of compounds comprising:
(a) from about 0.001% to about 99.99% of a beta-ionol analog compound having the formula: 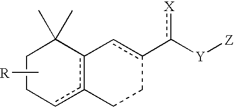
wherein “X” is a single or double bonded moiety comprising from 0 to about 12 substituted or unsubstituted carbon atoms; from 0 to about 2 heteroatoms, selected from substituted, unsubstituted, cycloalkyl and aromatic moieties of NH, S, O and combinations thereof; “Z” is a single, double, or triple bonded moiety containing from 0 to about 12 carbon atoms in a chain, optionally including a cycloalkyl or aromatic ring, both of which may be further substituted; “Y” is (CH₂)ₙ, wherein “n” is a variable having a value of from 0 to about 3; “R” is a group which may be substituted onto any ring if two or more are present and is selected from no greater than three independently selected substituted, unsubstituted, alkyl, cycloalkyl or aromatic moieties including CH₃, CH₂CH₃, NR₁R₂, SR, OR and combinations thereof;
(b) from about 99.99% to about 0.001% of a fatty acid analog compound having the formula: 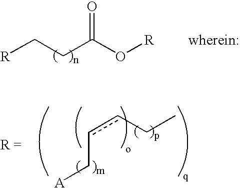
wherein “A” is selected from hydrogen, methyl, ethyl and mixtures thereof; further wherein “n”, “o”, “p”, and “m” are variables comprising a value of from 0 to about 8; further wherein methylene is saturated, unsaturated, substituted, unsubstituted, a constituent of a ring structure and combinations thereof; and
(c) optionally, a pharmaceutically acceptable carrier.

4. A beta-ionol analog compound for the beautification of mammalian skin, said compound being selected from the group consisting of:

| | | |
|---|---|---|
| 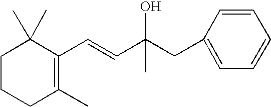 | 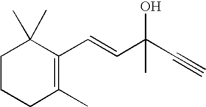 | 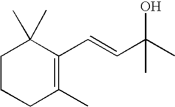 |
| 2-Methyl-1-phenyl-4- | 3-Methyl-1-(2,6,6- | 2-Methyl-4-(2,6,6- |
| (2,6,6-trimethyl- | trimethyl- | trimethyl-cyclohex- |
| cyclohex-1-enyl)-but-3- | cyclohex-1-enyl)- | 1-enyl)-but-3-en-2- |
| en-2-ol | pent-1-en-4-yn-3- | ol |
| | ol | |
| 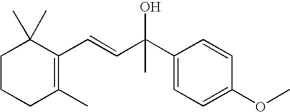 | 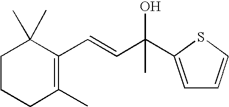 | 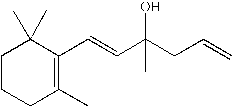 |
| 2-(4-Methoxy-phenyl)-4-(2,6,6- | 2-Thiophen-2-yl-4- | 3-Methyl-1-(2,6,6- |
| trimethyl-cyclohex-1-enyl)-but- | (2,6,6-trimethyl- | trimethyl-cyclohex- |
| 3-en-2-ol | cyclohex-1-enyl)-but- | 1-enyl)-hexa-1,5- |
| | 3-en-2-ol | dien-3-ol |
| 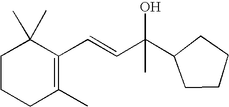 | 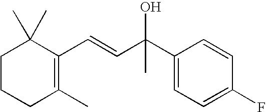 | 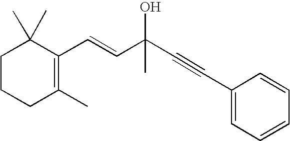 |
| 2-Cyclopentyl-4- | 2-(4-Fluoro-phenyl)-4-(2,6,6- | 3-Methyl-5-phenyl-1- |
| (2,6,6-trimethyl- | trimethyl-cyclohex-1-enyl)-but- | (2,6,6-trimethyl- |
| cyclohex-1-enyl)- | 3-en-2-ol | cyclohex-1-enyl)-pent- |
| but-3-en-2-ol | | 1-en-4-yn-3-ol |
| 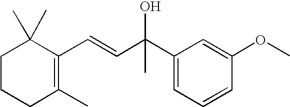 | 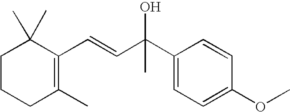 | 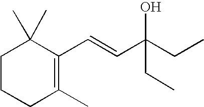 |
| 2-(3-Methoxy-phenyl)- | 2-(4-Methoxy-phenyl)- | 3-Ethyl-1-(2,6,6- |
| 4-(2,6,6-trimethyl- | 4-(2,6,6-trimethyl- | trimethyl-cyclohex-1- |
| cyclohex-1-enyl)-but-3- | cyclohex-1-enyl)-but-3- | enyl)-pent-1-en-3-ol |
| en-2-ol | en-2-ol | |
| 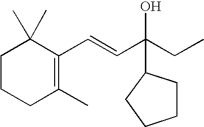 | 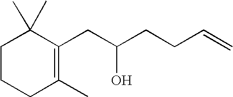 | 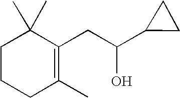 |
| 3-Cyclopentyl-1-(2,6,6- | 1-(2,6,6-Trimethyl- | 1-Cyclopropyl-2-(2,6,6- |
| trimethyl-cyclohex-1- | cyclohex-1-enyl)-hex-5- | trimethyl-cyclohex-1- |
| enyl)-pent-1-en-3-ol | en-2-ol | enyl)-ethanol |
| 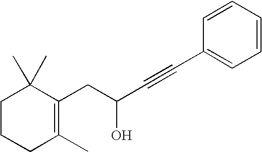 | 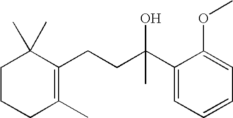 | 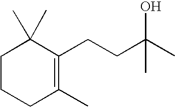 |
| 4-Phenyl-1-(2,6,6- | 2-(2-Methoxy-phenyl)- | 2-Methyl-4-(2,6,6- |
| trimethyl-cyclohex-1- | 4-(2,6,6-trimethyl- | trimethyl-cyclohex-1- |
| enyl)-but-3-yn-2-ol | cyclohex-1-enyl)- | enyl)-butan-2-ol |
| | butan-2-ol | |
| 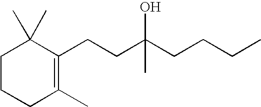 | 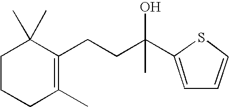 | 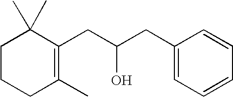 |
| 3-Methyl-1-(2,6,6- | 2-Thiophen-2-yl-4- | 1-Phenyl-3-(2,6,6- |
| trimethyl-cyclohex-1- | (2,6,6-trimethyl- | trimethyl-cyclohex-1- |
| enyl)-heptan-3-ol | cyclohex-1-enyl)- | enyl)-propan-2-ol |
| | butan-2-ol | |
| 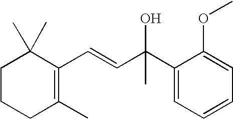 | 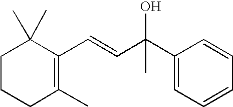 | 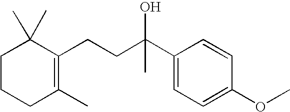 |
| 2-(2-Methoxy- | 2-Phenyl-4-(2,6,6- | 2-(4-Methoxy-phenyl)-4- |
| phenyl)-4-(2,6,6- | trimethyl-cyclohex- | (2,6,6-trimethyl-cyclohex-1- |
| trimethyl-cyclohex-1- | 1-enyl)-but-3-en-2- | enyl)-butan-2-ol |
| enyl)-but-3-en-2-ol | ol | |
| 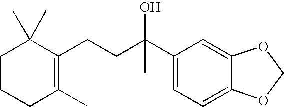 | 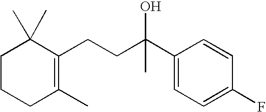 | 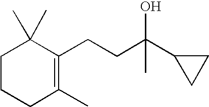 |
| 2-Benzo[1,3]dioxol-5- | 2-(4-Fluoro-phenyl)- | 2-Cyclopropyl-4- |
| yl-4-(2,6,6-trimethyl- | 4-(2,6,6-trimethyl- | (2,6,6-trimethyl- |
| cyclohex-1-enyl)-butan- | cyclohex-1-enyl)- | cyclohex-1-enyl)- |
| 2-ol | butan-2-ol | butan-2-ol |
| 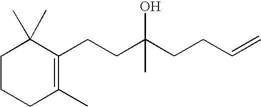 | 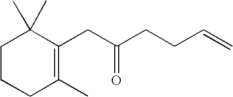 | 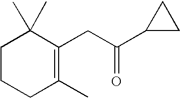 |
| 3-Methyl-1-(2,6,6-trimethyl- | 1-(2,6,6-Trimethyl- | 1-Cyclopropyl-2- |
| cyclohex-1-enyl)-hept-6- | cyclohex-1-enyl)-hex- | (2,6,6-trimethyl- |
| en-3-ol | 5-en-2-one | cyclohex-1-enyl)- |
| | | ethanone |
| 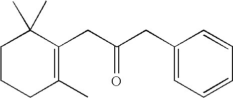 | 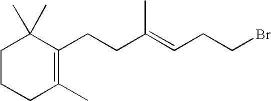 | 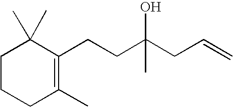 |
| 1-Phenyl-3-(2,6,6- | 2-(6-Bromo-3-methyl-hex- | 3-Methyl-1-(2,6,6- |
| trimethyl-cyclohex-1-enyl)- | 3-enyl)-1,3,3-trimethyl- | trimethyl-cyclohex-1- |
| propan-2-one | cyclohexene | enyl)-hex-5-en-3-ol |
| 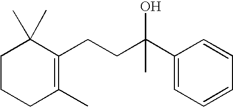 | 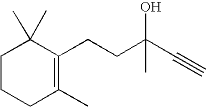 | 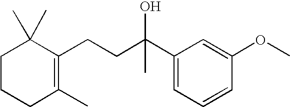 |
| 2-Phenyl-4-(2,6,6- | 3-Methyl-5-(2,6,6- | 2-(3-Methoxy-phenyl)-4- |
| trimethyl-cyclohex-1- | trimethyl- | (2,6,6-trimethyl-cyclohex-1- |
| enyl)-butan-2-ol | cyclohex-1-enyl)- | enyl)-butan-2-ol |
| | pent-1-yn-3-ol | |
| 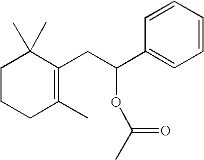 | 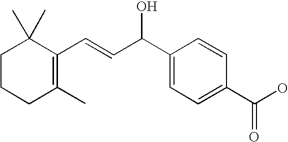 | 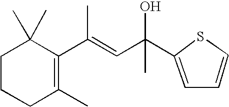 |
| Acetic acid 1-phenyl-2- | 4-[1-Hydroxy-3- | 2-Thiophen-2-yl-4- |
| (2,6,6-trimethyl- | (2,6,6-trimethyl- | (2,6,6-trimethyl- |
| cyclohex-1-enyl)- | cyclohex-1-enyl)- | cyclohex-1-enyl)- |
| ester | allyl]-benzoic acid | pent-3-en-2-ol |
| | methyl ester | |
| 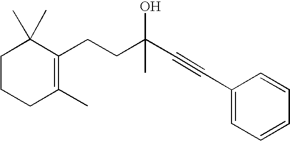 | 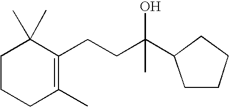 | 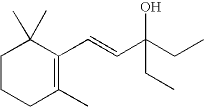 |
| 3-Methyl-1-phenyl-5- | 2-Cyclopentyl-4- | 3-Ethyl-1-(2,6,6- |
| (2,6,6-trimethyl- | (2,6,6-trimethyl- | trimethyl-cyclohex-1- |
| cyclohex-1-enyl)-pent- | cyclohex-1-enyl)- | enyl)-pent-1-en-3-ol |
| 1-yn-3-ol | | |
| 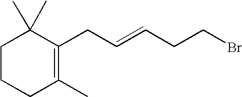 | 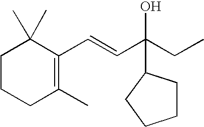 | 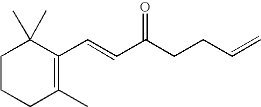 |
| 2-(5-Bromo-pent-2-enyl)- | 3-Cyclopentyl-1-(2,6,6- | 1-(2,6,6-Trimethyl- |
| 1,3,3-trimethyl- | trimethyl-cyclohex-1- | cyclohex-2-enyl)- |
| cyclohexene | enyl)-pent-1-en-3-ol | hepta-1,6-dien-3-one |
| 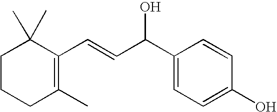 | 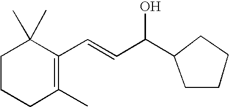 | 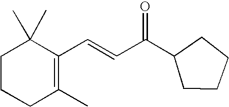 |
| 4-[1-Hydroxy-3-(2,6,6- | 1-Cyclopentyl-3-(2,6,6- | 1-Cyclopentyl-3-(2,6,6- |
| trimethyl-cyclohex-1- | trimethyl-cyclohex-1-enyl)- | trimethyl-cyclohex-1- |
| enyl)-allyl]-phenol | prop-2-en-1-ol | enyl)-propenone |
| 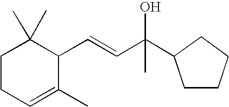 | 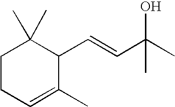 | 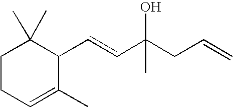 |
| 2-Cyclopentyl-4- | 2-Methyl-4-(2,6,6- | 3-Methyl-1-(2,6,6- |
| (2,6,6-trimethyl- | trimethyl-cyclohex-2- | trimethyl-cyclohex-2-enyl)- |
| cyclohex-2-enyl)-but- | enyl)-but-3-en-2-ol | hexa-1,5-dien-3-ol |
| 3-en-2-ol | | |
| 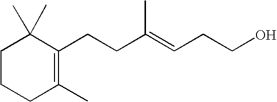 | 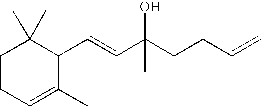 | 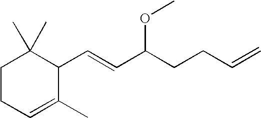 |
| 4-Methyl-6-(2,6,6- | 3-Methyl-1-(2,6,6- | 6-(3-Methoxy-hepta-1,6- |
| trimethyl-cyclohex-1- | trimethyl-cyclohex-2- | dienyl)-1,5,5-trimethyl- |
| enyl)-hex-3-en-1-ol | enyl)-hepta-1,6-dien- | cyclohexene |
| | 3-ol | |
| 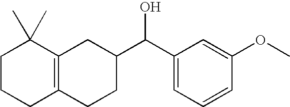 | 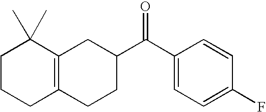 | 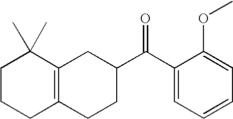 |
| (8,8-Dimethyl- | (8,8-Dimethyl- | (8,8-Dimethyl- |
| 1,2,3,4,5,6,7,8- | 1,2,3,4,5,6,7,8- | 1,2,3,4,5,6,7,8- |
| octahydro-naphthalen- | octahydro-naphthalen- | octahydro-naphthalen- |
| 2-yl)-(3-methoxy- | 2-yl)-(4-fluoro-phenyl)- | 2-yl)-(2-methoxy- |
| phenyl)-methanol | methanone | phenyl)-methanone |
| 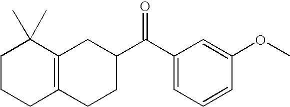 | 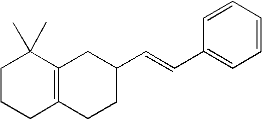 | 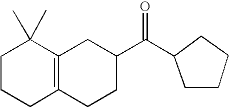 |
| (8,8-Dimethyl-1,2,3,4,5,6,7,8- | 7-[3-(4-Methoxy- | Cyclopentyl-(8,8- |
| octahydro-naphthalen-2-yl)-(3- | phenyl)-propa-1,2- | dimethyl-1,2,3,4,5,6,7,8- |
| methoxy-phenyl)-methanone | dienyl]-1,1-dimethyl-1,2,3,4,5,6,7,8- | octahydro-naphthalen- |
| | octahydro-naphthalene | 2-yl)-methanone |
| 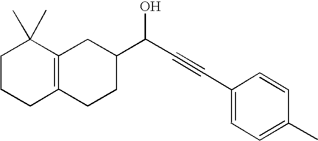 | 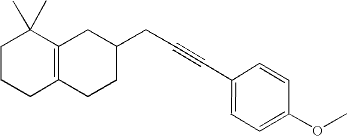 | 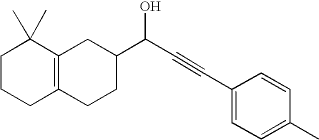 |
| 1-(8,8-Dimethyl- | 7-[3-(4-Methoxy-phenyl)-prop-2- | 4-[3-(8,8-Dimethyl- |
| 1,2,3,4,5,6,7,8- | ynyl]-1,1-dimethyl- | 1,2,3,4,5,6,7,8- |
| octahydro-naphthalen- | 1,2,3,4,5,6,7,8-octahydro- | octahydro-naphthalen- |
| 2-yl)-3-(4-methoxy- | naphthalene | 2-yl)-3-hydroxy-prop-1- |
| phenyl)-prop-2-yn-1-ol | ynyl]-phenol | |
| 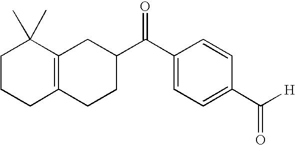 | 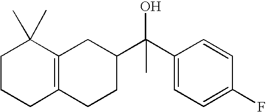 | 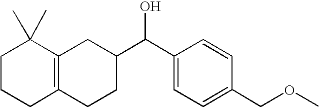 |
| 4-(8,8-Dimethyl- | 1-(8,8-Dimethyl- | (8,8-Dimethyl-1,2,3,4,5,6,7,8- |
| 1,2,3,4,5,6,7,8- | 1,2,3,4,5,6,7,8- | octahydro-naphthalen-2-yl)-(4- |
| octahydro- | octahydro-naphthalen- | methoxymethyl-phenyl)- |
| naphthalene-2- | 2-yl)-1-(4-fluoro- | methanol |
| carbonyl)-benzaldehyde | phenyl)-ethanol | |
| 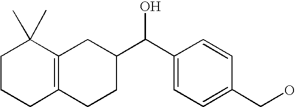 | 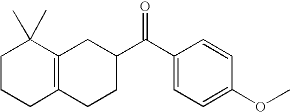 | 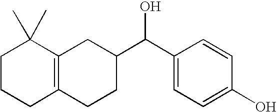 |
| (8,8-Dimethyl- | (8,8-Dimethyl- | 4-[(8,8-Dimethyl- |
| 1,2,3,4,5,6,7,8- | 1,2,3,4,5,6,7,8- | 1,2,3,4,5,6,7,8- |
| octahydro-naphthalen- | octahydro-naphthalen- | octahydro-naphthalen- |
| 2-yl)-(4-hydroxymethyl- | phenyl)-methanone | 2-yl)-hydroxy-methyl]- |
| phenyl)-methanol | | phenol |
| 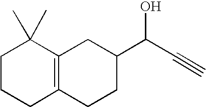 | 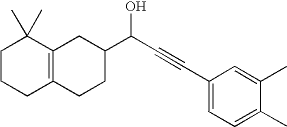 | 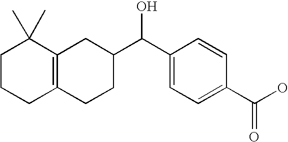 |
| 1-(8,8-Dimethyl-1,2,3,4,5,6,7,8- | 4-[3-(8,8-Dimethyl- | 4-[(8.8-Dimethyl- |
| octahydro-naphthalen-2-yl)- | 1,2,3,4,5,6,7,8- | 1,2,3,4,5,6,7,8- |
| prop-2-yn-1-ol | octahydro-naphthalen- | octahydro-naphthalen- |
| | 2-yl)-3-hydroxy-prop-1- | 2-yl)-hydroxy-methyl]- |
| | ynyl]-fluoro | benzoic acid isopropyl |
| | benzaldehyde | ester |
| 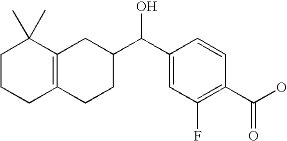 | 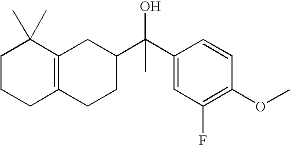 | 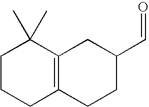 |
| 4-[(8,8-Dimethyl- | 1-(8,8-Dimethyl-1,2,3,4,5,6,7,8- | (−) Melafleur |
| 1,2,3,4,5,6,7,8- | octahydro-naphthalen-2-yl)-1- | |
| octahydro-naphthalen- | (4-methoxy-3-fluoro-phenyl)- | |
| 2-yl)-hydroxy-methyl]- | ethanol | |
| fluoro benzoic acid | | |
| 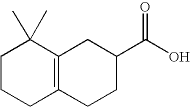 | 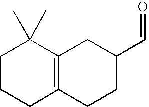 | 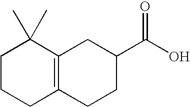 |
| (−) Melafleur acid | (+) Melafleur | (+) Melafleur acid |
| 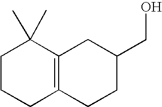 | 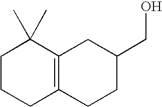 | 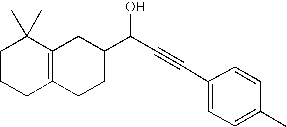 |
| (+) Melafleur alcohol | (−) Melafleur alcohol | 4-[3-(8,8-Dimethyl- |
| | | 1,2,3,4,5,6,7,8- |
| | | octahydro-naphthalen- |
| | | 2-yl)-3-hydroxy-prop-1- |
| | | ynyl]-benzaldehyde |
| 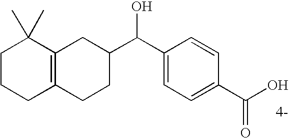 | 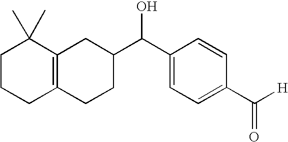 | 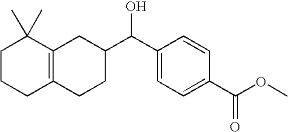 |
| [(8,8-Dimethyl- | 4-[(8,8-Dimethyl- | 4-[(8,8-Dimethyl- |
| 1,2,3,4,5,6,7,8-octahydro- | 1,2,3,4,5,6,7,8- | 1,2,3,4,5,6,7,8- |
| naphthalen-2-yl)-hydroxy- | octahydro-naphthalen-2- | octahydro-naphthalen- |
| methyl]-benzoic acid | yl)-hydroxy-methyl]- | 2-yl)-hydroxy-methyl]- |
| | | benzoic acid methyl |
| | | ester |
| 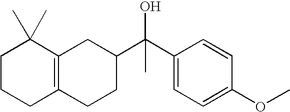 | 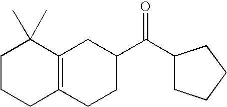 | 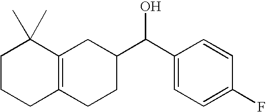 |
| 1-(8,8-Dimethyl- | Cyclopentyl-(8,8-dimethyl- | (8,8-Dimethyl- |
| 1,2,3,4,5,6,7,8- | 1,2,3,4,5,6,7,8-octahydro- | 1,2,3,4,5,6,7,8- |
| octahydro-naphthalen- | naphthalen-2-yl)-methanone | octahydro-naphthalen-2- |
| 2-yl)-1-(4-methoxy- | yl)-(4-fluoro-phenyl)- | |
| phenyl)-ethanol | methanol | |
| 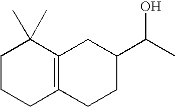 | 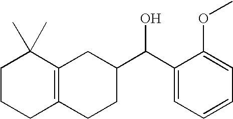 | 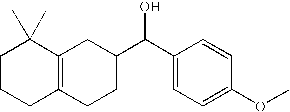 |
| 1-(8,8-Dimethyl- | (8,8-Dimethyl- | (8,8-Dimethyl- |
| 1,2,3,4,5,6,7,8- | 1,2,3,4,5,6,7,8- | 1,2,3,4,5,6,7,8-octahydro- |
| octahydro-naphthalen- | octahydro-naphihalen- | naphthalen-2-yl)-(4- |
| 2-yl)-ethanol | 2-yl)-(2-methoxy- | methoxy-phenyl)-methanol |
| | phenyl)-methanol | |
| 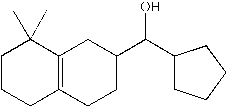 | 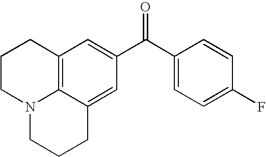 | 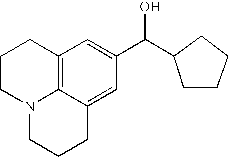 |
| Cyclopentyl-(8,8- | (4-Fluoro-phenyl)- | Cyclopentyl-(2,3,6,7- |
| dimethyl- | (2,3,6,7-tetrahydro- | tetrahydro-1H,5H- |
| 1,2,3,4,5,6,7,8- | 1H,5H-pyrido[3,2,1- | pyrido[3,2,1-ij]quinolin-9- |
| octahydro-naphthalen- | ij]quinolin-9-yl)- | yl)-methanol |
| 2-yl)-methanol | methanone | |
| 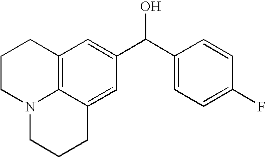 | 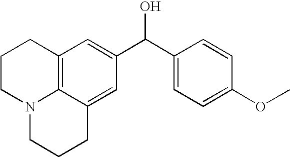 | 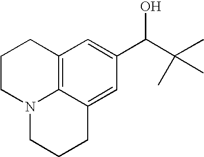 |
| (4-Fluoro-phenyl)- | (4-Methoxy-phenyl)- | 2,2-Dimethyl-1-(2,3,6,7- |
| (2,3,6,7-tetrahydro- | (2,3,6,7-tetrahydro- | tetrahydro-1H,5H- |
| 1H,5H-pyrido[3,2,1- | 1H,5H-pyrido[3,2,1- | pyrido[3,2,1-ij]quinolin-9- |
| ij]quinolin-9-yl)-methanol | ij]quinolin-9-yl)-methanol | yl)-propan-1-ol |
| 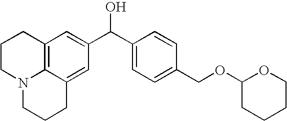 | 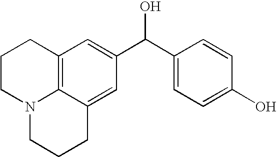 | 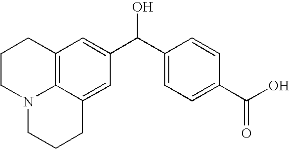 |
| [4-(Tetrahydro-pyran-2- | 4-[Hydroxy-(2,3,6,7- | 4-[Hydroxy-(2,3,6,7- |
| yloxymethyl)-phenyl]-(2,3,6,7- | tetrahydro-1H,5H- | tetrahydro-1H,5H- |
| tetrahydro-1H,5H-pyrido[3,2,1- | pyrido[3,2,1-ij]quinolin-9- | pyrido[3,2,1-ij]quinolin-9- |
| ij]quinolin-9-yl)-methanol | yl)-methyl]-phenol | yl)-methyl]-benzoic acid |
| 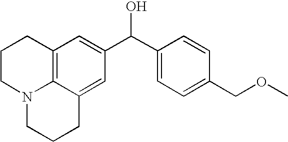 | 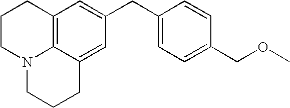 | 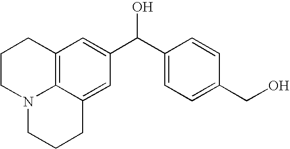 |
| (4-Methoxymethyl- | 9-(4-Methoxymethyl-benzyl)- | (4-Hydroxymethyl- |
| phenyl)-(2,3,6,7- | 2,3,6,7-tetrahydro-1H,5H- | tetrahydro-1H,5H- |
| tetrahydro-1H,5H- | pyrido[3,2,1-ij]quinoline | tetrahydro-1H,5H- |
| pyrido[3,2,1-ij]quinolin-9- | | pyrido[3,2,1-ij]quinolin-9- |
| yl)-methanol | | yl)-methanol |
| 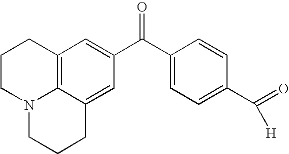 | 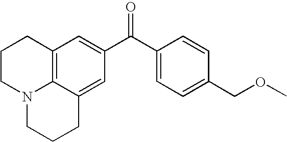 | 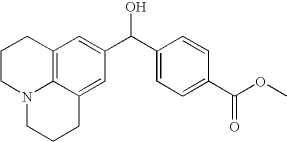 |
| 4-(2,3,6,7-Tetrahydro- | (4-Methoxymethyl- | 4-[Hydroxy-(2,3,6,7-tetrahydro- |
| 1H,5H-pyrido[3,2,1- | phenyl)-(2,3,6,7- | 1H,5H-pyrido[3,2,1-ij]quinolin- |
| ij]quinoline-9-carbonyl)- | tetrahydro-1H,5H- | 9-yl)-methyl]-benzoic acid |
| benzaldehyde | pyrido[3,2,1-ij]quinolin- | methyl ester |
| | 9-yl)-methanone | |
| 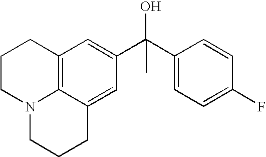 | 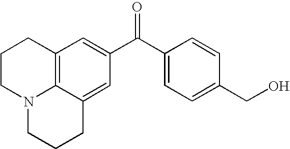 | 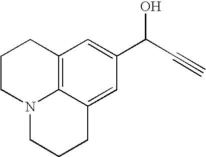 |
| 1-(4-Fluoro-phenyl)-1- | (4-Hydroxymethyl- | 1-(2,3,6,7-Tetrahydro- |
| (2,3,6,7-tetrahydro- | phenyl)-(2,3,6,7- | 1H,5H-pyrido[3,2,1- |
| 1H,5H-pyrido[3,2,1- | tetrahydro-1H,5H- | ij]quinolin-9-yl)-prop-2-yn- |
| ij]quinolin-9-yl)-ethanol | pyrido[3,2,1-ij]quinolin-9- | 1-ol |
| | yl)-methanone | |

and mixtures thereof.

5. A fatty acid analog compound for the beautification of mammalian skin, said compound being selected from the group consisting of: dihomolinolenic acid, alpha-linolenic acid, gamma linolenic acid, conjugated linolenic acid, arachidonic acid, conjugated linoleic acid, dihomo-gamma-linolenyl-ethanolamide, docosahexanenoic acid, docosapentaenoic acid, docosatetraenoic acid, docosatrienoic acid, linolaidic acid, stereodonic acid, docosenoic acid, oleic acid, steric acid, elaidic acid, myrstic acid, phytanic acid and combinations thereof.

6. A mixture of beta-ionol analog and fatty acid analog compounds for the beautification of mammalian skin, said mixture comprising at least one compound of claim 4 and at least one compound of claim 5.

7. A beautification product comprising the compound according to claim 1, the compound according to claim 2, or the mixture according to claim 3.

8. The beautification product according to claim 7, wherein said product takes the form of a wipe.

9. The beautification product according to claim 7, wherein said product takes the form of a facial tissue.

10. The beautification product according to claim 7, wherein said product takes the form of a topical skin care composition.

11. The topical skin care composition according to claim 10, further comprising a dermatologically acceptable carrier.

12. The beautification product of claim 7, further comprising a skin care active, wherein said skin care active is selected from the group consisting of: abrasives, absorbents, fragrances, pigments, colorings/colorants, essential oils, skin sensates, astringents, anti-acne agents, anti-caking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, opacifying agents, pH adjusters, propellants, reducing agents, sequestrants, skin bleaching and lightening agents, skin-conditioning agents, skin soothing and/or healing agents, skin treating agents, thickeners, vitamins, derivatives thereof and combinations thereof.

13. A method of beautifying mammalian skin, said method comprising the steps of topically applying the compounds according to claims 1, 2 or 3 to an area in need of treatment and, optionally, removing said compounds following their application.

14. A method of slowing the deterioration of mammalian skin, said method comprising the steps of topically applying the compounds according to claims 1, 2 or 3 to an area in need of treatment and, optionally, removing said compounds following their application.

15. A method of reducing the loss of function of mammalian skin, said method comprising the steps of topically applying the compounds according to claims 1, 2 or 3 to an area in need of treatment and, optionally, removing said compounds following their application.

16. A method of treating cancer, said method comprising the steps of applying the mixture according to claim 3 to an area in need of treatment and, optionally, removing said mixture following its application.

17. A novel compound useful for treating cancer and disorders of the skin wherein said compound has the following structure: 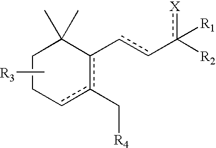
wherein “X” is a heteroatom selected from the group consisting of substituted and unsubstituted O, N and S; wherein O, N and S may be singly- or doubly bonded to the molecule, with the caveat that when the heteroatom is doubly bonded, then there is no R₂;
further wherein R₁, R₂ and R₃ and R₄ are independently selected from the group consisting of: H, lower alkyl chain of from 0 to about 6 member atoms, monocyclic, bicyclic and aromatic rings, wherein R₁, R₂ and R₃ and R₄ are substituted or unsubstituted; with the caveat that neither R₁ nor R₂ may be methyl or hydrogen when “X” is a hydroxyl moiety, and with the caveat that when “X” is allylic, R₁ may not be H when R₂ is lower alkyl, phenyl or alkynyl.

18. A novel compound for treating cancer and disorders of mammalian skin, said compound having the following structure: 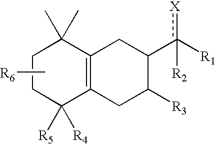
wherein “X” is H, or a heteroatom selected from the group consisting of: N, O, P, S and mixtures thereof: wherein N, O, P and S are substituted or unsubstituted and singly or doubly bonded to the molecule, with the caveat that when a heteroatom is doubly bonded, then there is no R₂;
further wherein R₁ and R₂ are independently selected from the group consisting of: H, lower alkyl chain of from 0 to about 6 member atoms, monocyclic, bicyclic, and aromatic rings, wherein said member atoms are substituted or unsubstituted; with the caveat that R₁ and R₂ may not simultaneously be H or methyl when “X” is OH and that R₁ is H when “X” is 0 in the absence of R₂;
further wherein R₃, R₄, R₅, and R₆ are independently selected from the group consisting of: H, lower alkyl chain of from 0 to about 6 member atoms, monocyclic, bicyclic, and aromatic rings, wherein said member atoms may be substituted or unsubstituted; with the caveat that R₃ is not methyl and that R₄ and R₅ are not simultaneously methyl.

19. A novel compound useful for treating cancer and disorders of the skin, said compound having the following structure: 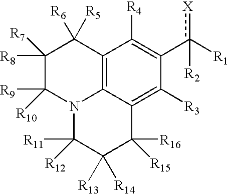
wherein “X” is CH₂, or a heteroatom selected from the group consisting of: N, O, S substituted or unsubstituted and singly or doubly bonded to the molecule, with the caveat that when said heteroatom is doubly bonded, then there is no R₂;
further, when “X” is a ketone moiety and R₃ and R₄ are simultaneously H, then R₁ may not be H, Me, ethyl, CH₂CH₂Cl, CH₂BrMe, OH, CH₂NH₂, CH₂CHPh, (CO)Me, or (CO)Ph; when “X” is OH and R₂, R₃ and R₄ are simultaneously H, then R₁ may not be ethyl or (CO)OEt;
further wherein R₁ and R₂ are independently selected from the group consisting of: H, lower alkyl chain of from 0 to about 6 member atoms, monocyclic, bicyclic, and aromatic rings, wherein said member atoms are substituted or unsubstituted; with the caveat that R₁ and R₂ may not be comprised of aniline moieties substituted or unsubstituted; with the caveat that R₁ and R₂ may not both be aromatic rings; with the caveat that R₁ and R₂ may not be, contain, be substituted by, or be contained within nitrogen-containing rings, and may not be joined in a ring with “X” via an ester linkage; further with the caveat that R₁ and R₂ may not contain an acid anhydride moiety;
further wherein R₃ and R₄ are independently selected from the group consisting of: H, lower alkyl chain of from 0 to about 6 member atoms, monocyclic, and aromatic rings, wherein said member atoms may not be substituted; with the caveat that when “X” is a double bonded 0, R₁, R₃, and R₇-R₁₄ are H, and R₅,R₆,R₁₅,R₁₆ are Me, then R₄ cannot be H, OH, OMe, or OCH₂Ome; with the caveat that when “X” is a double bonded 0, R₁, R₃, and R₅—R₁₆ are H, then R₄ cannot be H, OH, OMe, OEt, OPh, or OAc; and in such instance if R₁ is Me and R₃ is OH, then R₄ cannot be Me, CF₃, Ph, CH₂CH₂Ph;
further wherein R₅-R₁₆ are independently selected from the group consisting of: H, lower alkyl chain of from 0 to about 3 member atoms; with the caveat that R₅-R₁₆ may not represent moieties that produce unstable compounds; with the caveat that when R₅-R₁₀ and R₁₃-R₁₆ are H then R₁₁ and R₁₂ may not combine to form a ketone; further wherein any geminal group of R₅-R₁₆ may be combined to form a cyclopropyl moiety or an exocyclic methylene.

20. The novel compound according to claim 17, wherein said compound is selected from the group consisting of:

| | | |
|---|---|---|
| 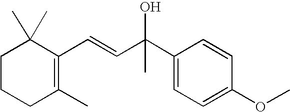 | 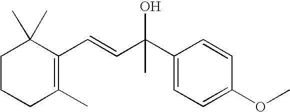 | 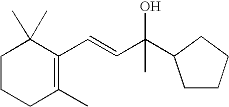 |
| 2-(4-Methoxy-phenyl)-4- | 2-(4-Methoxy-phenyl)- | 2-Cyclopentyl-4-(2,6,6- |
| (2,6,6-trimethyl-cyclohex-1- | 4-(2,6,6-trimethyl- | trimethyl-cyclohex-1- |
| enyl)-but-3-en-2-ol | cyclohex-1-enyl)-but-3- | enyl)-but-3-en-2-ol |
| | en-2-ol | |
| 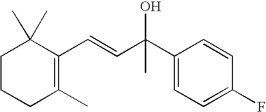 | 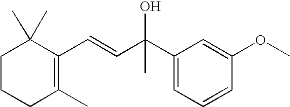 | 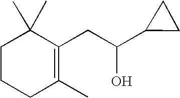 |
| 2-(4-Fluoro-phenyl)-4- | 2-(3-Methoxy-phenyl)-4- | 1-Cyclopropyl-2-(2,6,6- |
| (2,6,6-trimethyl-cyclohex- | (2,6,6-trimethyl-cyclohex-1- | trimethyl-cyclohex-1- |
| 1-enyl)-but-3-en-2-ol | enyl)-but-3-en-2-ol | enyl)-ethanol |
| 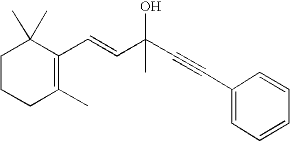 | 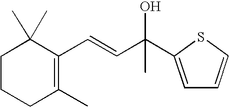 | 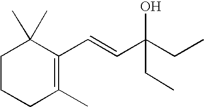 |
| 3-Methyl-5-phenyl-1- | 2-Thiophen-2-yl-4-(2,6,6- | 3-Ethyl-1-(2,6,6-trimethyl- |
| (2,6,6-trimethyl-cyclohex- | trimethyl-cyclohex-1- | cyclohex-1-enyl)-pent-1-en- |
| 1-enyl)-pent-1-en-4-yn-3- | enyl)-but-3-en-2-ol | 3-ol |
| ol | | |
| 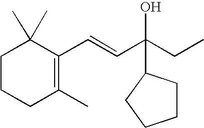 | 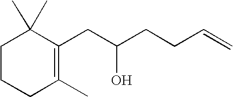 | 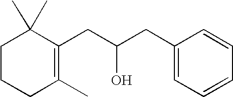 |
| 3-Cyclopentyl-1-(2,6,6- | 1-(2,6,6-Trimethyl- | 1-Phenyl-3-(2,6,6- |
| trimethyl-cyclohex-1- | cyclohex-1-enyl)-hex-5- | trimethyl-cyclohex-1- |
| enyl)-pent-1-en-3-ol | en-2-ol | enyl)-propan-2-ol |
| 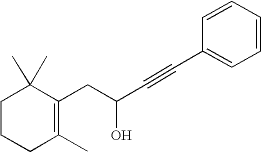 | 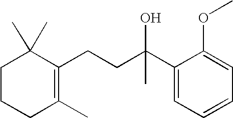 | 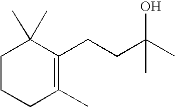 |
| 4-Phenyl-1-(2,6,6-trimethyl- | 2-(2-Methoxy-phenyl)- | 2-Methyl-4-(2,6,6- |
| cyclohex-1-enyl)-but-3-yn-2- | 4-(2,6,6-trimethyl- | trimethylcyclohex-1- |
| ol | cyclohex-1-enyl)-butan- | enyl)-butan-2-ol |
| | 2-ol | |
| 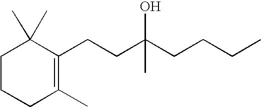 | 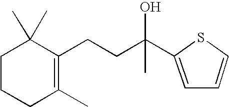 | 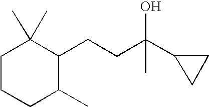 |
| 3-Methyl-1-(2,6,6- | 2-Thiophen-2-yl-4-(2,6,6- | 2-Cyclopropyl-4-(2,6,6- |
| trimethyl-cyclohex-1- | trimethyl-cyclohex-1-enyl)- | trimethyl-cyclohexyl)- |
| enyl)-heptan-3-ol | butan-2-ol | butan-2-ol |
| 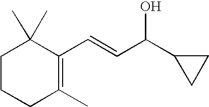 | 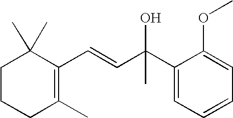 | 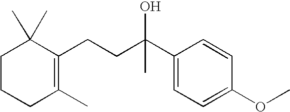 |
| 1-Cyclopropyl-3-(2,6,6- | (2-(2-Methoxy-phenyl)-4- | 2-(4-Methoxy-phenyl)-4- |
| trimethyl-cyclohex-1- | (2,6,6-trimethyl-cyclohex- | (2,6,6-trimethyl-cyclohex-1- |
| enyl)-prop-2-en-1-ol | 1-enyl)-but-3-en-2-ol | enyl)-butan-2-ol |
| 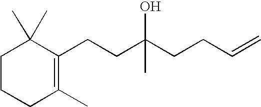 | 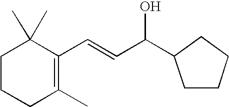 | 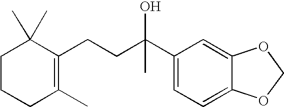 |
| 3-Methyl-1-(2,6,6- | 1-Cyclopentyl-3-(2,6,6- | 2-Benzo[1,3]dioxol-5-yl-4- |
| trimethyl-cyclohex-1- | trimethyl-cyclohex-1- | (2,6,6-trimethyl-cyclohex- |
| enyl)-hept-6-en-3-ol | enyl)-prop-2-en-1-ol | 1-enyl)-butan-2-ol |
| 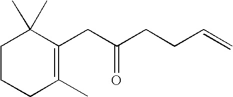 | 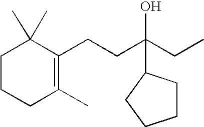 | 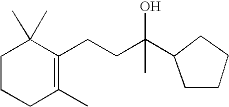 |
| 1-(2,6,6-Trimethyl-cyclohex- | 3-Cyclopentyl-1-(2,6,6- | 2-Cyclopentyl-4-(2,6,6- |
| 1-enyl)-hex-5-en-2-one | trimethyl-cyclohex-1- | trimethyl-cyclohex-1- |
| | enyl)-pentan-3-ol | enyl)-butan-2-ol |
| 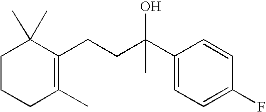 | 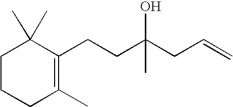 | 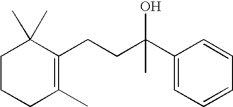 |
| 2-(4-Fluoro-phenyl)-4- | 3-Methyl-1-(2,6,6-trimethyl- | 2-Phenyl-4-(2,6,6- |
| (2,6,6-trimethyl-cyclohex- | cyclohex-1-enyl)-hex-5-en-3- | trimethyl-cyclohex-1- |
| 1-enyl)-butan-2-ol | ol | enyl)-butan-2-ol |
| 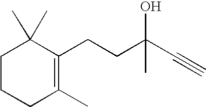 | 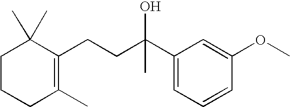 | 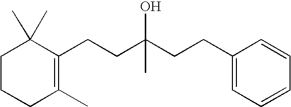 |
| 3-Methyl-5-(2,6,6- | 2-(3-Methoxy-phenyl)-4- | 3-Methyl-1-phenyl-5-(2,6,6- |
| trimethyl-cyclohex-1- | (2,6,6-trimethyl-cyclohex- | trimethyl-cyclohex-1-enyl)- |
| enyl)-pent-1-yn-3-ol | 1-enyl)-butan-2-ol | pentan-3-ol |
| 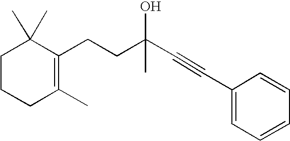 | 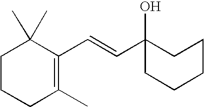 | 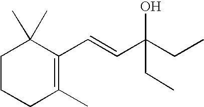 |
| 3-Methyl-1-phenyl-5- | 1-[2-(2,6,6-Trimethyl- | 3-Ethyl-1-(2,6,6-trimethyl- |
| (2,6,6-trimethyl- | cyclohex-1-enyl)-vinyl]- | cyclohex-1-enyl)-pent-1- |
| cyclohex-1-enyl)-pent- | cyclohexanol | en-3-ol |
| 1-yn-3-ol | | |
| | |
|---|---|
| 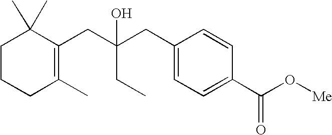 | 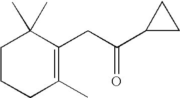 |
| 4-[2-Hydroxy-2-(2,6,6- | 1-Cyclopropyl-2-(2,6,6- |
| trimethyl-cyclohex-1- | trimethyl-cyclohex-1- |
| enylmethyl)-butyl]-benzoic | enyl)-ethanone |
| acid methyl ester | |
| | | |
|---|---|---|
| 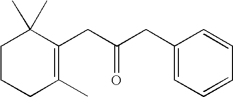 | 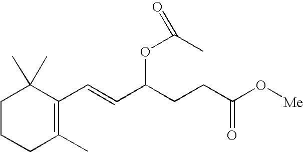 | 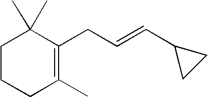 |
| 1-Phenyl-3-(2,6,6- | 4-Acetoxy-6-(2,6,6- | 2-(3-Cyclopropyl-allyl)-1,3,3- |
| trimethyl-cyclohex-1- | trimethyl-cyclohex-1- | trimethyl-cyclohexene |
| enyl)-propan-2-one | enyl)-hex-5-enoic acid | |
| | methyl ester | |
| 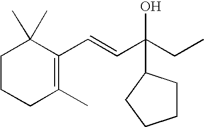 | 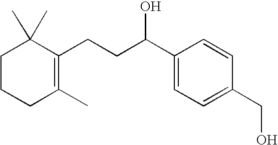 | 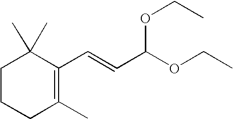 |
| 3-Cyclopentyl-1-(2,6,6- | 1-(4-Hydroxymethyl- | 2-(3,3-Diethoxy- |
| trimethyl-cyclohex-1- | phenyl)-3-(2,6,6- | propenyl)-1,3,3-trimethyl- |
| enyl)-pent-1-en-3-ol | trimethyl-cyclohex-1- | cyclohexene |
| | enyl)-propan-1-ol | |
| 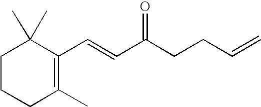 | 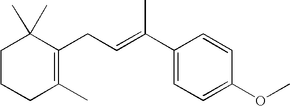 | 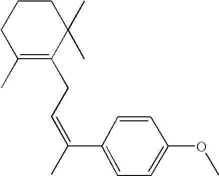 |
| 1-(2,6,6-Trimethyl-cyclohex- | 1-Methoxy-4-[1-methyl- | 1-Methoxy-4-[1-methyl-3- |
| 2-enyl)-hepta-1,6-dien-3-one | 3-(2,6,6-trimethyl- | (2,6,6-trimethyl-cyclohex- |
| | cyclohex-1-enyl)- | 1-enyl)-propenyl]- |
| | propenyl]-benzene | benzene |
| | |
|---|---|
| 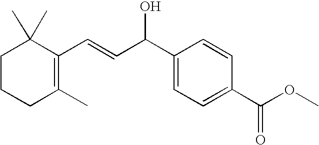 | 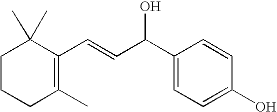 |
| 4-[1-Hydroxy-3-(2,6,6- | 4-[1-Hydroxy-3-(2,6,6- |
| trimethyl-cyclohex-1- | trimethyl-cyclohex-1-enyl)- |
| enyl)allyl]-benzoic acid | allyl]-phenol |
| methyl ester | |
| | | |
|---|---|---|
| 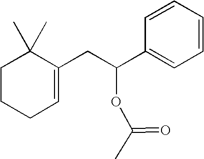 | 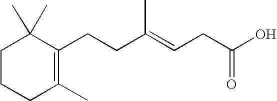 | 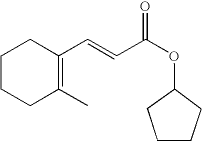 |
| Acetic acid 1-phenyl-2- | 4-Methyl-6-(2,6,6- | 3-(2,6,6-Trimethyl- |
| (2,6,6-trimethyl- | trimethyl-cyclohex-1- | cyclohex-1-enyl)-acrylic |
| cyclohex-1-enyl)-ethyl | enyl)-hex-3-enoic acid | acid cyclopentyl ester |
| ester | | |
| | |
|---|---|
| 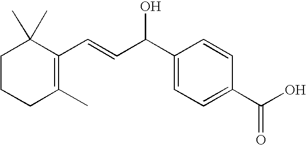 | 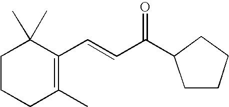 |
| 4-[1-Hydroxy-3-(2,6,6- | 1-Cyclopentyl-3-(2,6,6- |
| trimethyl-cyclohex-1-enyl)- | trimethyl-cyclohex-1- |
| allyl]-benzoic acid | enyl)-propenone |
| 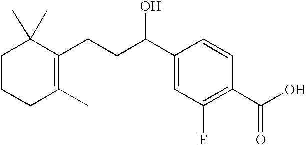 | 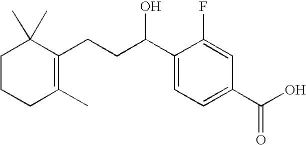 |
| 2-Fluoro-4-[1-hydroxy-3- | 3-Fluoro-4-[1-hydroxy-3- |
| (2,6,6-trimethyl-cyclohex- | (2,6,6-trimethyl-cyclohex- |
| 1-enyl)-propyl]-benzoic | 1-enyl)-propyl]-benzoic |
| acid | acid |
| and combinations thereof. | |

21) The novel compound according to claim 18, wherein said compound is selected from the group consisting of:

| | | |
|---|---|---|
| 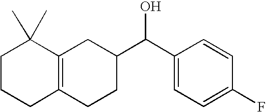 | 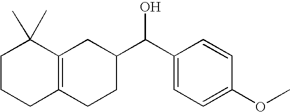 | 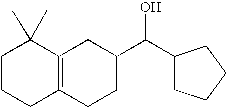 |
| (8,8-Dimethyl- | (8,8-Dimethyl-1,2,3,4,5,6,7,8- | Cyclopentyl-(8,8- |
| 1,2,3,4,5,6,7,8- | octahydro-naphthalen-2- | dimethyl-1,2,3,4,5,6,7,8- |
| octahydro-naphthalen- | yl)-(4-methoxy-phenyl)- | octahydro-naphthalen-2- |
| 2-yl)-(4-fluoro-phenyl)- | methanol | |
| methanol | | |
| | |
|---|---|
| 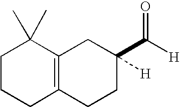 | 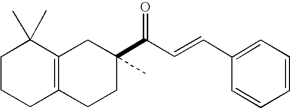 |
| (−) and (+) melafleur | (1-(8,8-Dimethyl- |
| (each separately | 1,2,3,4,5,6,7,8- |
| claimed) | octahydro-naphthalen- |
| | 2-yl)-3-phenyl-propenone |
| 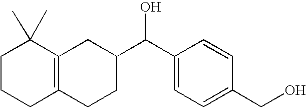 | 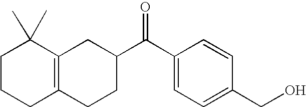 |
| (8,8-Dimethyl- | (8,8-Dimethyl- |
| 1,2,3,4,5,6,7,8- | 1,2,3,4,5,6,7,8-octahydro- |
| octahydro-naphthalen-2- | naphthalen-2-yl)- |
| yl)-(4-hydroxymethyl- | (4-hydroxymethyl- |
| phenyl)-methanol | phenyl)-methanone |
| 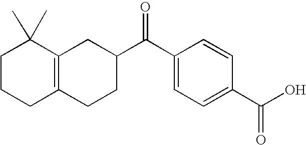 | 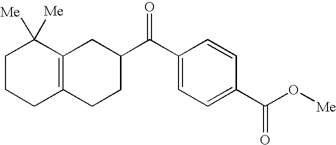 |
| 4-(8,8-Dimethyl- | 4-(8,8-Dimethyl- |
| 1,2,3,4,5,6,7,8- | 1,2,3,4,5,6,7,8- |
| octahydro- | octahydro-naphthalene- |
| naphthalene- | 2-carbonyl)-benzoic |
| 2-carbonyl)-benzoic | acid methyl ester |
| acid | |
| 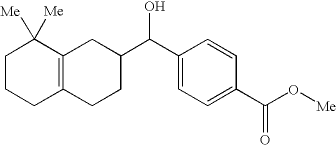 | 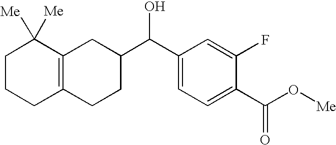 |
| 4-[(8,8-Dimethyl- | 4-[(8,8-Dimethyl- |
| 1,2,3,4,5,6,7,8- | 1,2,3,4,5,6,7,8- |
| octahydro-naphthalen-2- | octahydro-naphthalen-2- |
| yl)-hydroxy-methyl]- | yl)-hydroxy-methyl]-2- |
| benzoic acid methyl | fluoro-benzoic acid |
| ester | methyl ester |
| 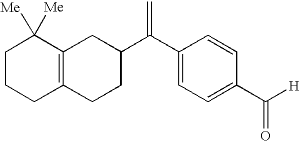 | 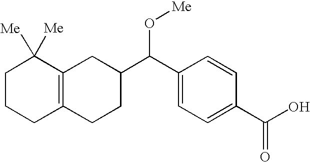 |
| 4-[1-(8,8-Dimethyl- | 4-[(8,8-Dimethyl- |
| 1,2,3,4,5,6,7,8- | 1,2,3,4,5,6,7,8- |
| octahydro- | octahydro-naphthalen- |
| naphthalen-2-yl)- | 2-yl)-methoxy-methyl]- |
| vinyl]-benzaldehyde | benzoic acid |
| 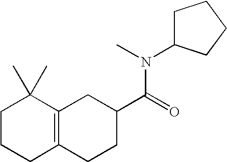 | 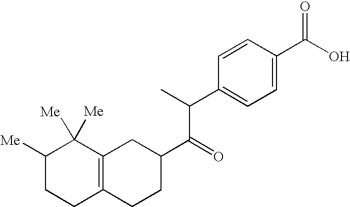 |
| 8,8-Dimethyl- | 4-[1-Methyl-2-oxo-2- |
| 1,2,3,4,5,6,7,8- | (7,8,8-trimethyl |
| octahydro-naphthalene- | 1,2,3,4,5,6,7,8- |
| 2-carboxylic acid | octahydro-naphthalen-2- |
| cyclopentyl-methyl | yl)-ethyl]-benzoic acid |
| amide | |
| 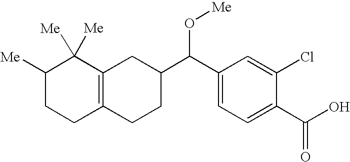 | 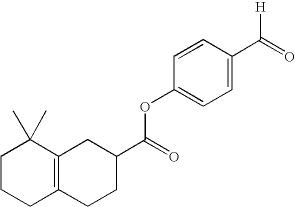 |
| 2-Chloro-4- | 8,8-Dimethyl |
| [methoxy-(7,8,8- | 1,2,3,4,5,6,7,8- |
| trimethyl-1,2,3,4,5,6,7,8- | octahydro- |
| octahydro- | naphthalene-2- |
| naphthalene-2-yl)- | carboxylic acid |
| methyl]-benzoic acid | |
| 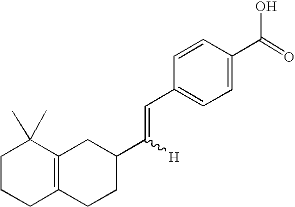 | 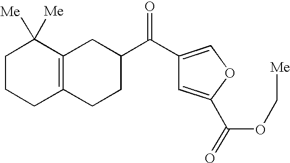 |
| 4-[2-(8,8-Dimethyl- | 4-(8,8-Dimethyl- |
| 1,2,3,4,5,6,7,8- | 1,2,3,4,5,6,7,8- |
| octahydro-naphthalen- | octahydro-naphthalene- |
| 2-yl) | 2-carbonyl)-furan-2- |
| 2-yl)-vinyl]-benzoic acid | carboxylic acid ethyl ester |
| 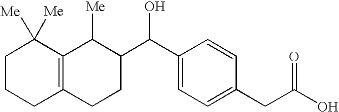 | 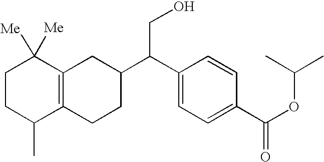 |
| {4-[Hydroxy-(1,8,8- | 4-[2-Hydroxy-1-(5,8,8- |
| trimethyl-1,2,3,4,5,6,7,8- | trimethyl-1,2,3,4,5,6,7,8- |
| octahydro-naphthalen-2-yl)- | octahydro-naphthalen- |
| methyl]-phenyl}-acetic acid | 2-yl)-ethyl]-benzoic |
| | acid isopropyl ester |
| 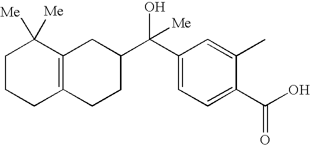 | 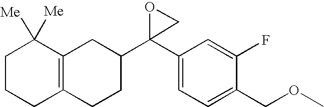 |
| 4-[1-(8,8-Dimethyl- | 2-(8,8-Dimethyl- |
| 1,2,3,4,5,6,7,8- | 1,2,3,4,5,6,7,8- |
| octahydro-naphthalen-2- | octahydro-naphthalen-2- |
| yl)-1-hydroxy-ethyl]-2- | yl)-2-(3-fluoro-4- |
| methyl- | methoxymethyl-phenyl)- |
| benzoic acid | oxirane |
| 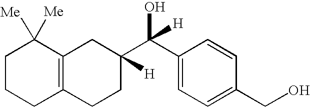 | 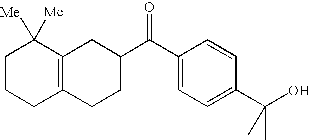 |
| (−) and (+) (8,8- | (8,8-Dimethyl-1,2,3,4,5,6,7,8- |
| Dimethyl-1,2,3,4,5,6,7,8- | octahydro-naphthalen-2- |
| octahydro-naphthalen-2-yl)-(4- | yl)-[4-(1-hydroxy-1- |
| hydroxymethyl-phenyl)-methanol | methyl-ethyl)-phenyl]- |
| | metha none |
| 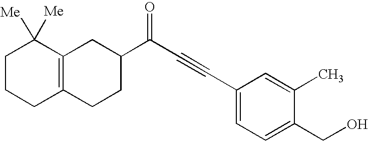 | 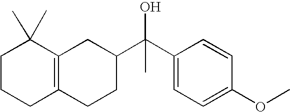 |
| 1-(8,8-Dimethyl- | 1-(8,8-Dimethyl- |
| 1,2,3,4,5,6,7,8- | 1,2,3,4,5,6,7,8- |
| octahydro-naphthalen-2- | octahydro-naphthalen-2- |
| yl)-3-(4-hydroxymethyl- | yl)-1-(4-methoxy- |
| 3-methyl-phenyl)-propy | phenyl)-ethanol |
| none | |
| 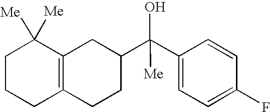 | 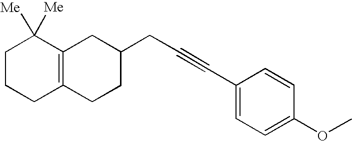 |
| 1-(8,8-Dimethyl-1,2,3,4,5,6,7,8- | 7-[3-(4-Methoxy- |
| octahydro-naphthalen-2-yl)-1- | phenyl)-prop-2-ynyl]- |
| (4-fluoro-phenyl)-ethanol | 1,1-dimethyl-1,2,3,4,5,6,7,8- |
| | octahydro-naphthalene |
| 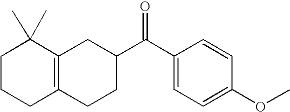 | 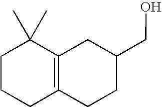 |
| (8,8-Dimethyl-1,2,3,4,5,6,7,8- | (+) and (−) melafleur |
| octahydro-naphthalen-2- | alcohols |
| yl)-(4-methoxy-phenyl)- | (Separately claimed) |
| methanone | |
| 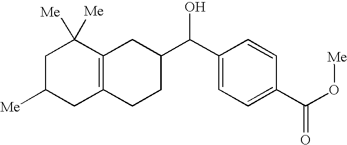 | |
| 4-[Hydroxy-(6,8,8-trimethyl- | |
| 1,2,3,4,5,6,7,8-octahydro- | |
| naphthalen-2-yl)- methyl]-benzoic acid | |
| methyl ester | |
| and combinations thereof. | |

22) The novel compound according to claim 19, wherein said compound is selected from the group consisting of:

| | | |
|---|---|---|
| 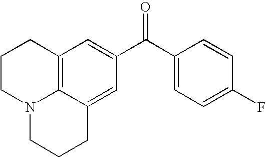 | 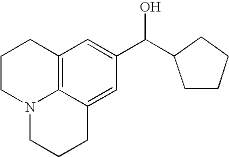 | 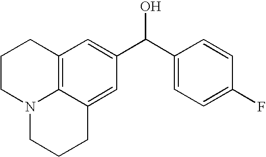 |
| (4-Fluoro-phenyl)-(2,3,6,7- | Cyclopentyl-(2,3,6,7- | (4-Fluoro-phenyl)- |
| tetrahydro-1H,5H- | tetrahydro-1H,5H-pyrido[3,2,1- | (2,3,6,7-tetrahydro- |
| pyrido[3,2,1-ij]quinolin-9-yl)- | ij]quinolin-9-yl)-methanol | 1H,5H-pyrido[3,2,1- |
| methanone | | ij]quinolin-9-yl)-methanol |
| | |
|---|---|
| 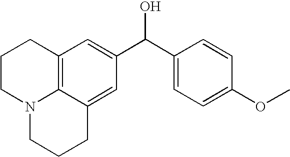 | 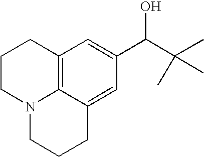 |
| (4-Methoxy-phenyl)- | 2,2-Dimethyl-1-(2,3,6,7- |
| (2,3,6,7-tetrahydro-1H,5H- | tetrahydro-1H,5H- |
| pyrido[3,2,1-ij]quinolin-9-yl)- | pyrido[3,2,1-ij]quinolin-9-yl)- |
| methanol | propan-1-ol |
| 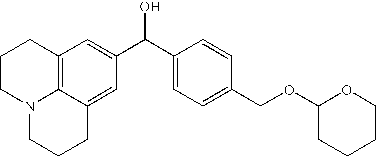 | 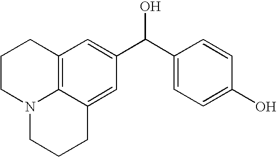 |
| [4-(Tetrahydro-pyran-2- | 4-[Hydroxy-(2,3,6,7- |
| yloxymethyl)-phenyl]- | tetrahydro-1H,5H- |
| (2,3,6,7-tetrahydro-1H,5H- | pyrido[3,2,1-ij]quinolin-9- |
| pyrido[3,2,1-ij]quinolin-9-yl)- | yl)-methyl]-phenol |
| methanol | |
| 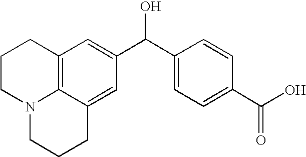 | 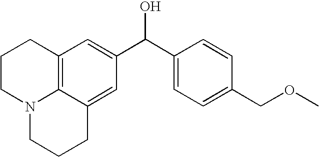 |
| 4-[Hydroxy-(2,3,6,7- | (4-Methoxymethyl-phenyl)- |
| tetrahydro-1H,5H- | (2,3,6,7-tetrahydro-1H,5H- |
| pyrido[3,2,1-ij]quinolin-9-yl)- | pyrido[3,2,1-ij]quinolin-9-yl)- |
| methyl]-benzoic acid | methanol |
| 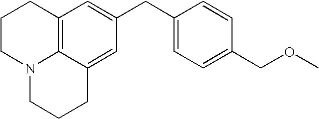 | 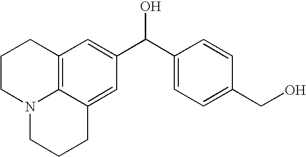 |
| 9-(4-Methoxymethyl-benzyl)- | (4-Hydroxymethyl- |
| 2,3,6,7-tetrahydro-1H,5H- | phenyl)-(2,3,6,7- |
| pyrido[3,2,1-ij]quinoline | tetrahydro-1H,5H- |
| | pyrido[3,2,1-ij]quinoline-9- |
| | yl)-methanol |
| 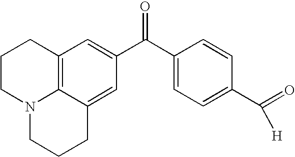 | 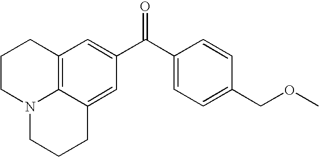 |
| 4-(2,3,6,7-Tetrahydro- | (4-Methoxymethyl-phenyl)- |
| 1H,5H-pyrido[3,2,1- | (2,3,6,7-tetrahydro-1H,5H- |
| ij]quinoline-9-carbonyl)- | pyrido[3,2,1-ij]quinolin-9-yl)- |
| benzaldehyde | methanone |
| 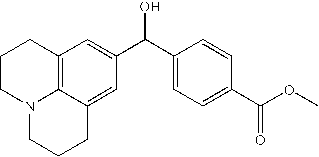 | 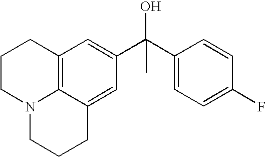 |
| 4-[Hydroxy-(2,3,6,7- | 1-(4-Fluoro-phenyl)-1- |
| tetrahydro-1H,5H- | (2,3,6,7-tetrahydro- |
| pyrido[3,2,1-ij]quinolin-9-yl)- | 1H,5H-pyrido[3,2,1- |
| methyl]-benzoic acid methyl | ij]quinolin-9-yl)-ethanol |
| ester | |
| 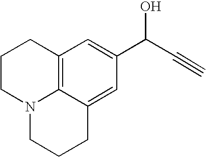 | 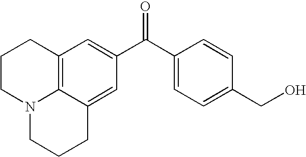 |
| 1-(2,3,6,7-Tetrahydro- | (4-Hydroxymethyl-phenyl)- |
| 1H,5H-pyrido[3,2,1- | (2,3,6,7-tetrahydro-1H,5H- |
| ij]quinolin-9-yl)-prop-2-yn-1-ol | pyrido[3,2,1-ij]quinolin-9-yl)- |
| | methanone |
| 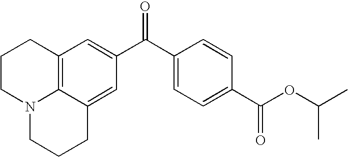 | 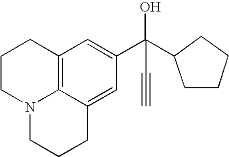 |
| 4-(2,3,6,7-Tetrahydro- | 1-Cyclopentyl-1-(2,3,6,7- |
| 1H,5H-pyrido[3.2.1- | tetrahydro-1H,5H- |
| ij]quinoline-9-carbonyl)- | pyrido[3,2,1-ij]quinolin-9- |
| benzoic acid isopropyl ester | yl)-prop-2-yn-1-ol |
| 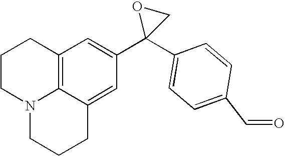 | 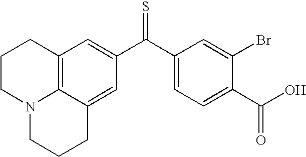 |
| 4-[2-(2,3,6,7-Tetrahydro- | 2-Bromo-4-(2,3,6,7- |
| 1H,5H-pyrido[3,2,1- | tetrahydro-1H,5H- |
| ij]quinolin-9-yl)-oxiranyl]- | pyrido[3,2,1-ij]quinoline-9- |
| benzaldehyde | carbothioyl)-benzoic acid |
| 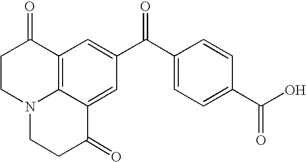 | 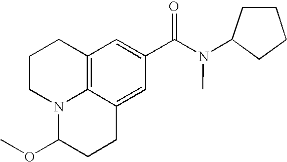 |
| 4-(1,7-Dioxo-2,3,6,7- | 3-Methoxy-2,3,6,7- |
| tetrahydro-1H,5H- | tetrahydro-1H,5H- |
| pyrido[3,2,1-ij]quinoline-9- | pyrido[3,2,1-ij]quinoline- |
| carbonyl)-benzoic acid | 9-carboxylic acid |
| | cyclopentyl-met |
| | hyl-amide |
| 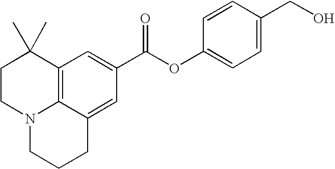 | 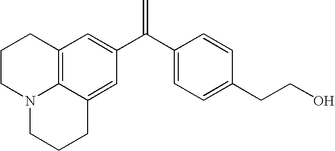 |
| 1,1-Dimethyl-2,3,6,7- | 2-{4-[1-(2,3,6,7-Tetrahydro- |
| tetrahydro-1H,5H- | 1H,5H-pyrido[3,2,1- |
| pyrido[3,2,1-ij]quinoline-9- | ij]quinolin-9-yl)-vinyl]- |
| carboxylic acid 4- | phenyl}-ethanol |
| hydroxymet | |
| hyl-phenyl ester | |
| 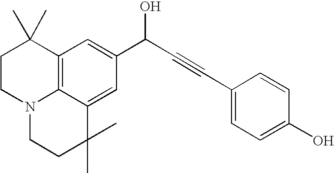 | 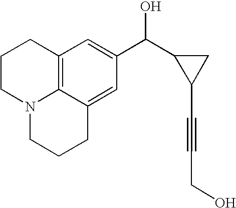 |
| 4-[3-Hydroxy-3-(1,1,7,7- | 3-{2-[Hydroxy-(2,3,6,7- |
| tetramethyl-2,3,6,7- | tetrahydro-1H,5H- |
| tetrahydro-1H,5H- | pyrido[3,2,1-ij]quinolin-9- |
| pyrido[3,2,1-ij]quinolin-9-yl)- | yl)-methyl]-cyclopropyl}- |
| pr op-1-ynyl]-phenol | propynol |
| 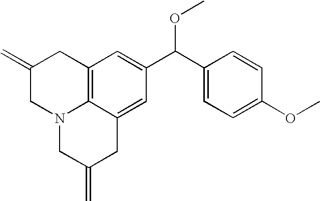 | 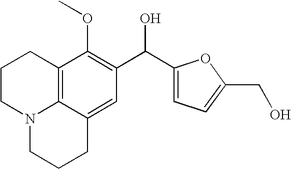 |
| 9-[Methoxy-(4-methoxy- | (5-Hydroxymethyl-furan-2- |
| phenyl)-methyl]-2,6- | yl)-(8-methoxy-2,3,6,7- |
| dimethylene-2,3,6,7- | tetrahydro-1H,5H- |
| tetrahydro-1H,5H- | pyrido[3,2,1-ij]quinolin-9-yl)- |
| pyrido[3,2,1-ij]quinoline | methanol |
| 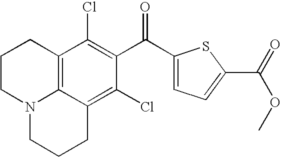 | 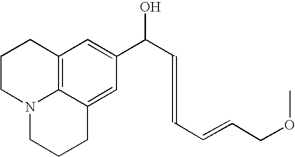 |
| 5-(8,10-Dichloro-2,3,6,7- | 7-Methoxy-1-(2,3,6,7- |
| tetrahydro-1H,5H- | tetrahydro-1H,5H- |
| pyrido[3,2,1-ij]quinoline-9- | pyrido[3,2,1-ij]quinolin-9- |
| carbonyl)-thiophene-2- | yl)-hepta-2,3,5-trien-1-ol |
| carboxylic acid methyl ester | |
| 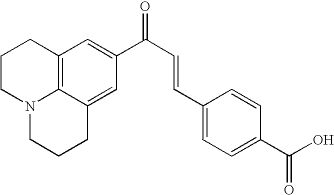 | |
| 4-[3-Oxo-3-(2,3,6,7- | |
| tetrahydro-1H,5H- | |
| pyrido[3,2,1-ij]quinolin-9-yl)- | |
| propenyl]-benzoic acid | |

and combinations thereof.

23. A method of treating contact dermatitis, said method comprising the steps of delivering the compounds according to claims 1, 2 or 3 to an area in need of treatment, and optionally, removing said compounds following their delivery.

24. A method of treating allergic dermatitis, said method comprising the steps of delivering the compounds according to claims 1, 2 or 3 to an area in need of treatment, and optionally, removing said compounds following their delivery.

25. A method of inducing differentiation and/or proliferation of RXR-containing mammalian tissue in need of stimulation, said method comprising the steps of delivering the compounds of claims 1, 2 or 3 to an area of RXR-containing mammalian tissue in need of stimulation, and optionally, removing said compounds following their delivery.

26. A method of beautifying mammalian skin, said method comprising the steps of delivering an RXR ligand to an area of mammalian skin in need of beautification, and optionally, removing said RXR ligand following its delivery.

27. A method of beautifying mammalian skin, said method comprising the steps of delivering bexarotene to an area of mammalian skin in need of beautification, and optionally, removing said bexarotene following its delivery.

28. A method of beautifying mammalian skin, said method comprising the steps of delivering an RXR ligand and a fatty acid analog compound of claim 2 to an area of mammalian skin in need of beautification, and optionally, removing said RXR ligand and fatty acid analog compounds following their delivery.

29. A method of beautifying mammalian skin, said method comprising the steps of delivering bexarotene and a fatty acid analog compound of claim 2 to an area of mammalian skin in need of beautification, and optionally, removing said bexarotene and fatty acid analog compound following their delivery.

30. The method according to claim 28 or claim 29, wherein beautifying refers to an act selected from the group consisting of: removing fine lines, removing wrinkles, repairing photo damaged skin, repairing aged skin, improving skin surface texture, reducing skin hyperpigmentation, improving skin sagging, repairing damage from disease and combinations thereof.

31. The method according to claim 30, wherein said disease is selected from the group consisting of: allergic dermatitis, contact dermatitis, lymphoma, diabetes, gastrointestinal disorders and combinations thereof.
